(19) United States
(12) Patent Application Publication
(10) Pub. No.: US 2014/0187519 A1
(43) Pub. Date: Jul. 3, 2014

(54) BIOMARKERS FOR PREDICTING MAJOR ADVERSE EVENTS

(71) Applicant: The Board Of Trustees Of The Leland Stanford Junior University, Palo Alto, CA (US)

(72) Inventors: John P. Cooke, Palo Alto, CA (US); Nicholas J. Leeper, Menlo Park, CA (US); Kevin Nead, Palo Alto, CA (US)

(73) Assignee: The Board Of Trustees Of The Leland Stanford Junior University, Palo Alto, CA (US)

(21) Appl. No.: 14/095,053

(22) Filed: Dec. 3, 2013

Related U.S. Application Data
(60) Provisional application No. 61/746,341, filed on Dec. 27, 2012.
(60) Provisional application No. 61/826,261, filed on May 22, 2013.

(51) Int. Cl. G01N 33/68

(52) U.S. Cl. CPC G01N 33/6893
USPC 514/165; 435/7.92; 514/327; 514/312; 514/263.36

(57) ABSTRACT

Provided herein are diagnostic markers and uses thereof for predicting if a subject is at risk of a major adverse event. In particular, one aspect provided herein relates to methods to determine if a subject is at risk of having a major adverse effect by measuring at least 2, or at least 3 of the biomarkers beta 2 microglobulin, c-reactive protein and cystatin C.

### CROSS-REFERENCE TO RELATED APPLICATIONS

This application claims benefit under 35 U.S.C. §119(e) of U.S. Provisional Patent Application No. 61/746,341 filed Dec. 27, 2012, and of U.S. Provisional Patent Application No. 61/826,261 filed May 22, 2013, the contents of each of which are incorporated herein by reference in their entireties.

### GOVERNMENT SUPPORT

This invention was made with Government Support under Grant Number K12HL087746, awarded by the National Institutes of Health. The Government has certain rights to this invention.

### FIELD OF THE INVENTION

The present invention relates generally to the field of diagnostics, more particularly, the present invention generally relates to diagnostic markers for predicting major adverse side events, such as heart attack, stroke and death in a subject.

### BACKGROUND OF THE INVENTION

Clinical evaluation for determination of disease severity and risk of major adverse cardiac events (MACE), e.g., mortality due to heart failure, may not always be apparent. The decision whether to treat a subject aggressively or conservatively, or to admit the subject as an inpatient or to send them home, may sometimes be made solely on a physician's clinical assessment or “gut feeling” as to the individual's actual condition. A formula for determining a subject's likelihood of an adverse outcome, e.g., mortality, transplantation, and/or readmission, would significantly enhance the physician's ability to make informed treatment decisions, improve patient care and reduce overall healthcare costs.

Heart attack is the single leading cause of death (see world wide web at: “americanheart.org”). One of every five deaths in the United States results from a heart attack. In 2004, there were 452,327 deaths in the United States due to heart attack resulting from approximately 1,200,000 new and recurrent cardiovascular attacks.

Stroke is the third leading cause of death in the United States (see world wide web at: “americanheart.org”). Stroke killed 150,147 people in 2004 resulting from approximately 700,000 new and recurrent cerebrovascular events. Stroke is a leading cause of serious, long-term disability in the United States. About 5,700,000 stroke survivors are alive today in the United States. 2,400,000 are males and 3,300,000 are females.

Both heart attack and certain types of stroke can result from the rupture of vulnerable atherosclerotic plaque (Naghavi, et al., Circulation 108: 1664-72 & 108:1772-8, 2003). At present, the risk of having a heart attack or stroke is assessed in the general population by considering certain clinical and biochemical risk factors (Wilson et al., Circulation 97:1837-47, 1998; ATP III, JAMA 285:2486-97, 2001), but these characteristics do not fully explain cardiovascular risk (Khot, et al., JAMA 290:898-904, 2003; Greenland, et al., JAMA 290:891-7, 2003).

Peripheral arterial disease (PAD) is a highly morbid condition and is a common atherosclerotic disease of the non-coronary, non-cerebral vasculature that affects 8-12 million people in the US (Criqui et al., Circulation. 1985; 71: 510-, Hirsch et al., JAMA. 2001; 286: 1317-24.). The public health impact of this disease is significant, due to its high prevalence in our aging population^{1, 3} and the increased risk for negative clinical outcomes. Thus, although highly prevalent, PAD remains highly undiagnosed in our society; for instance, over half of patients identified as having PAD in the PARTNERS study were newly diagnosed (Hirsch et al., JAMA. 2001; 286: 1317-24). While such a low rate of diagnosis might not have been surprising for a study conducted in non-specialty primary care clinics, it is now known that diagnosis rates are no better for patients cared for by cardiologists (Sadrzadeh Rafie et al., asc Med. 2010; 15: 443-50). PAD may remain undiagnosed because as few as 11% of patients exhibit the classic overt symptomology of intermittent claudication (Hirsch et al., JAMA. 2001; 286: 1317-24) or because of technical issues with ABI measurements. Because PAD remains highly undiagnosed, PAD patients do not receive optimal treatment and are exposed to higher risks for adverse outcomes. (McDermott et al., J Gen Intern Med. 1997; 12: 209-15, Anand et al., Can J Cardiol. 1999; 15: 1259-63, Oka et al., Vasc Med. 2005; 10: 91-6, Valentijn et al., Curr Vasc Pharmacol. 2012; 10: 725-7). Compared to patients with coronary artery disease (CAD) or cerebrovascular disease (CVD), patients with PAD actually have higher rates of all-cause mortality and major cardiovascular events.⁸ Although there are many similarities between CAD and PAD patients, genetic, metabolomic and epidemiological differences suggest subtle pathophysiological distinctions between these two conditions.

Conventional risk factors for coronary artery disease are also associated with PAD and have been the basis of current risk prediction models (Duval et al., Vasc Med. 2012; 17: 342-51). While useful for risk stratification, these risk prediction algorithms do not fully capture an individual's likelihood of having disease. Ideally, risk prediction models would incorporate a range of independent factors, extending beyond just clinical risk factors to include circulating biomarkers reflective of environmental exposures, as well as genetic markers indicative of heritable risk.

Methods to identify PAD have been pursued, but have thus far had only modest success. PAD is indicated by an ABI<0.9, an index that can be obtained using a hand-held Doppler and a blood pressure cuff, or with automated oscillometry. However, not all practitioners have access to the necessary equipment and trained staff, which may contribute to the poor rate of diagnosis (Fung et al., Vasc Med. 2008; 13: 217-24; Fowkes et al., J Epidemiol Community Health. 1988; 42: 128-33). Because many patients remain undiagnosed and consequently do not receive optimal treatment, they are known to have higher risks for adverse outcomes (McDermott et al., J Gen Intern Med. 1997; 12: 209-15, Anand et al., Can J Cardiol. 1999; 15: 1259-63, Oka et al., Vasc Med. 2005; 10: 91-6, Valentijn et al., Curr Vasc Pharmacol. 2012; 10: 725-7, Steg et al., JAMA. 2007; 297: 1197-206). These data highlight the need for more accurate methods of PAD diagnosis and risk prediction.

Accordingly, improved methods of risk classification are needed to enhance proper PAD diagnosis and treatment. Thus, an ability to predict the future occurrence of PAD and subsequent heart attack or stroke could be improved, individuals with such risk could be targeted for preventative measures and the overall incidence of these leading causes of death could be reduced.

Measurement of multiple proteins and metabolites in the blood of an individual offers the prospect of a “window” into that individual's biochemical status and might provide a better indication of the status of his or her cardiovascular system and the likelihood of that subject experiencing a future heart attack or stroke (Vasan, Circulation 113:2335-62, 2006).

The development and refinement of risk stratification tools and prognostication models have and will continue to significantly impact the treatment and prevention of cardiovascular disease. To date, these efforts have largely aimed to reclassify intermediate-risk patients either upwards into a subset where an intervention becomes clearly indicated or downwards into a subset where it is likely that they can safely abstain from treatment. However, it is becoming increasingly clear that individuals felt to be at high-risk can similarly be re-stratified and may particularly benefit from appropriately intensified therapy (Ambrose et al., Vulnerable plaques and patients: improving prediction of future coronary events. Am J Med 2010; 123:10-16). Especially with more expensive or invasive cardiovascular therapies, it is important to develop new tools to identify those truly at highest risk and most suitable for intervention and/or more intensive risk factor modification.

Thus, there is a significant need in the art for a satisfactory biomarkers to identify subjects at high risk of experiencing a major adverse event, and which will aid the prognosis of a person's risk of a major adverse event, and can be used to monitor the subject more closely to prevent such a major adverse event, or treat the subject prophylactically with a more intensive medical therapy to prevent the major adverse event. In particular, reliable and cost-effective methods and compositions are needed to allow for diagnosis and/or prediction of major adverse events. In particular, evidence-based therapies are available to reduce the risk of death from cardiovascular disease, yet many patients go untreated. Accordingly, novel methods are needed to identify those at highest risk of cardiovascular death.

### SUMMARY OF THE INVENTION

The present invention generally relates to diagnostic markers for predicting a major adverse effect, such as a heart attack, stroke or death in a subject by measuring at least one, or at least 2, or at least three biomarkers, including beta 2 microglobulin, c-reactive protein (CRP) and cystatin C.

The inventors have previously identified a set of biomarkers that are preferentially expressed in patients with peripheral arterial disease (PAD), a group of patients at particularly elevated risk of major clinical events such as myocardial infarction and stroke (Wilson et al., Beta2-microglobulin as a biomarker in peripheral arterial disease: proteomic profiling and clinical studies. Circulation 2007; 116:1396-1403). Additionally, U.S. Pat. Nos. 7,998,743 and 8,227,201 (which are incorporated herein in their entirety by reference) disclose the use of beta-2-microglobulin (also known as “B2M” or “β2M”), CRP and cystatin-c for diagnosis of peripheral artery disease, but unlike the present study, the '743 and '201 patents do not teach that this combination of biomarkers can be used to identify a subject at risk of a major adverse event.

Herein, the inventors have determined that these biomarkers improve risk modeling in a cohort of patients undergoing coronary angiography, and identify a subject at risk of having a major adverse event.

In particular, the inventors measured the biomarkers beta-2-microglobulin, cystatin C, C-reactive protein (CRP) and plasma glucose levels at baseline in a cohort of participants undergoing coronary angiography, and discovered that they predicted the cardiovascular mortality. Adjusted Cox proportional-hazards models were used to determine whether the biomarkers predicted all-cause and cardiovascular mortality. Additionally, improvements in risk reclassification and discrimination were evaluated by calculating the net reclassification improvement (NRI), C-index and the integrated discrimination improvement with the addition of the biomarkers to a baseline model of risk factors for cardiovascular disease and death. During a median follow-up period of 5.6 years, there were 78 deaths among 470 participants. All biomarkers independently predicted future all-cause and cardiovascular mortality. A significant improvement in risk reclassification was observed for all-cause (NRI, 35.8%; P=0.004) and cardiovascular (NRI, 61.9%; P=0.008) mortality compared to the baseline risk factors model. Additionally, the inventors discovered that there was a significantly increased risk discrimination with a C-index of 0.777 (change in C-index [ΔC], 0.057; 95% CI, 0.016-0.097) and 0.826 (ΔC, 0.071; 95% CI, 0.010-0.133) for all-cause and cardiovascular mortality respectively. Improvements in risk discrimination were further supported using the integrated discrimination improvement index. In conclusion, the inventors demonstrate that beta-2-microglobulin, cystatin C and C-reactive protein (CRP), and plasma glucose levels predict mortality and improve risk reclassification and discrimination for a high-risk cohort undergoing coronary angiography.

In one aspect, the present invention relates to a method of identifying a subject at risk of a major adverse event, the method comprising detecting in a biological sample taken from the subject presenting a symptom of an acute cardiac event, or BMI of 25-30 or greater than 30, for the level of at least three biomarkers selected from beta-2-microglobulin, c-reactive protein (CRP) and cystatin C or plasma glucose level, wherein combination of the levels of beta-2-microglobulin, c-reactive protein (CRP) and cystatin C equal to, or above a threshold reference level for each of beta-2-microglobulin, c-reactive protein (CRP) and cystatin C indicates that the subject is at risk of a major adverse event.

Another aspect of the present invention relates to a method comprising: (a) assaying a biological sample from the subject to determine the levels of beta-2-microglobulin, c-reactive protein (CRP) and cystatin C; (b) determining a level of beta-2-microglobulin, c-reactive protein (CRP) and cystatin C or plasma glucose level that is equal to, or above a reference threshold level for each biomarker; and (c) diagnosing the subject as in need of treatment or therapy to prevent the occurrence of a major adverse event.

Another aspect of the present invention relates to a method for treating a human subject with a risk of a major adverse event, comprising administering a treatment or therapy to prevent the occurrence of a major adverse event to a human subject who is determined to have a level of beta-2-microglobulin, c-reactive protein (CRP), cystatin C or plasma glucose level equal to, or above a reference threshold level for each biomarker.

Another aspect of the present invention relates to an assay comprising: (a) measuring the levels of antibodies that are reactive to at least three biomarkers selected from beta-2 microglobulin, C-reactive protein (CRP), and cystatin C and/or optionally measuring plasma glucose levels in a biological sample obtained from a subject who has a body mass index (BMI) of 25 or greater for determining the likelihood of the subject having a major adverse event; and (b) comparing the level of the antibodies of the least three biomarkers in the biological sample with a reference antibody level for each of beta-2 microglobulin, C-reactive protein (CRP) and cystatin C and/or reference plasma glucose level, wherein a detectable increase of each antibody for each biomarker in the biological sample above the reference antibody level and/or reference plasma glucose level indicates the likelihood of the subject at risk of having a major adverse event.

In some embodiments, the levels of beta-2-microglobulin, c-reactive protein (CRP), cystatin C and/or plasma glucose levels are measured in a biological sample obtained from a subject who has fasted, or whom has had a defined caloric or dietary intake at a period of time before the blood was obtained from the subject. In some embodiments, the biological sample is a blood-based biological sample, or urine sample.

In some embodiments, the levels of beta-2-microglobulin, C-reactive protein (CRP), cystatin C and/or plasma glucose levels are measured using an antibody, antibody fragment or protein-binding molecule or other protein-binding probe, and in some embodiments, an antibody, antibody fragment or protein-binding molecule or other protein-binding probe is bound to a solid support. In some embodiments, the levels of beta-2-microglobulin, c-reactive protein (CRP), cystatin C and/or plasma glucose levels are measured using an immunoassay, such as an ELISA.

Another aspect of the present invention relates to the discovery that a polymorphism at the rs10757269 allele of the chromosome 9p21 (in particular, G-allele of rs10757269) is a cardiovascular-risk and indicates a risk of PAD (peripheral Arterial Disease) a group of patients at particularly elevated risk of major adverse cardiovascular event (MACE), such as, but not limited to, myocardial infarction and stroke. In particular, the inventors have demonstrated that the panel of biomarkers (e.g., the level of beta-2-microglobulin, c-reactive protein (CRP) and cystatin C, and plasma glucose equal to, or above a reference threshold level for each biomarker and the G-allele of rs10757269), is reflective of heritable risk and proteomic information (e.g., a level of beta-2-microglobulin, c-reactive protein (CRP) and cystatin C, and plasma glucose above a predefined threshold level) integrates environmental exposures, and can be used to predict the presence or absence of PAD better than any current or established risk models.

In some embodiments, the present invention relates to an assay to determine if the subject is at risk of a major cardiac event (MAE), the comprising: (a) subjecting a biological sample obtained from a subject with a Body Mass Index (BMI) of 25 or greater to at least one genotyping assay that determines the genotype of the allele at the rs10757269 loci; (b) determining the genotype of the allele at the rs10757269 loci; and (c) selecting a treatment regimen for the subject where the subject has at least one G-allele at the rs10757269 loci and is at risk of a major cardiac event, and not selecting the treatment regimen for the subject where the subject does not have at least one G-allele at the rs10757269 loci.

Another aspect of the present invention relates to an assay to determine if the subject is at risk of peripheral artery disease (PAD), comprising: (a) subjecting a biological sample obtained from a subject with a Body Mass Index (BMI) of 25 or greater to at least one genotyping assay that determines the genotype of the allele at the rs10757269 loci; (b) determining the genotype of the allele at the rs10757269 loci; and (c) selecting a treatment regimen for the subject where the subject has at least one G-allele at the rs10757269 loci and is at risk of PAD, and not selecting the treatment regimen for the subject where the subject does not have at least one G-allele at the rs10757269 loci.

In some embodiments, the subject has a genotype of G/A or G/G at the rs10757269 loci. In some embodiments, the treatment regimen is selected from any of the combination of: healthy diet, increased exercise, increased weight loss, medication to decrease blood pressure, and aspirin. In some embodiments, the treatment regimen for the subject where the subject has at least one G-allele at the rs10757269 loci is selected from any combination of treatments in the group consisting of: an exercise program; control of blood pressure, decreased sugar intake, and/or decreased lipid levels, cessation of smoking, and administration of drug therapies including the administration of aspirin (with or without dipyridamole), clopidogrel, cilostazol, and/or pentoxifylline.

In some embodiments, the subject who has at least one G-allele at the rs10757269 loci is determined to have a major adverse event in the next 12 months or earlier, for example, a major adverse event which is, for example, but not limited to a stroke, heart attack or death. In some embodiments, a major adverse event is a major adverse cardiovascular or cerebrovascular event (MACCE), for example, but not limited to a recurrence of an initial cardiac event, angina, decompensation of heart failure, admission for cardiovascular disease (CVD), mortality due to CVD, and transplant.

In some embodiments, a biological sample is a blood-based sample or a urine sample, or a serum, plasma or blood sample. In some embodiments, the biological sample is obtained from a subject that has been hospitalized after an acute cardiac event. In some embodiments, the genotyping of the rs10757269 is performed on a human subject, e.g., a subject who has been diagnosed with heart failure, or a subject has a body mass index (BMI) of 25 to 29, or a BMI of greater or equal to 30, and/or has pulmonary disorder or a liver disorder.

In some embodiments, the genotyping assay used to determine the allele of the rs10757269 loci is selected from any or a combination in the group consisting of: PCR-based assays, RT-PCR, nucleic acid hybridization, sequence analysis, TaqMan SNP genotyping probes, microarrays, direct or indirect sequencing, restriction site analysis, hybridization based genotyping assays, gel migration assays, antibody assays, fluorescent polarization, mass spectroscopy, allele-specific PCR, single-strand conformational polymorphism (SSCP) analysis, heteroduplex analysis, oligonucleotide ligation, PCR-RFLP, allele-specific amplification (ASA), single-molecule dilution (SMD), coupled amplification and sequencing (CAS), Restriction enzyme analysis, restriction fragment length polymorphism (RFLP), ligation based assays, single base extension (or minisequencing), MALDI-TOF, and homogenous assays.

In some embodiments, the genotyping assay detects a G-allele at position 27 of SEQ. ID NO: 1, or a C-allele in the complementary nucleic acid sequence of SEQ. ID NO: 1. In some embodiments, the genotyping assay comprises an allele-specific oligonucleotide (ASO) probe which specifically hybridizes to a G-allele at position 27 of SEQ. ID NO: 1, or a C-allele in the complementary nucleic acid sequence of SEQ ID NO: 1. In some embodiments, the allele-specific oligonucleotide (ASO) probe is a nucleic acid probe and comprises a detectable signal or a means to generate a detectable signal. In some embodiments, the genotyping assay comprises at least one probe flanking position 27 of SEQ ID NO: 1. In some embodiments, the genotyping assay comprises at least one allele-specific oligonucleotide (ASO) primer that specifically hybridizes to the G-allele at position 27 of SEQ ID NO: 1.

In another embodiment, the treatment regimen for the subject where the subject has at least one G-allele at the rs10757269 loci is selected from any suitable treatment for peripheral arterial disease (PAD).

Another aspect of the present invention relates to methods, assays and systems comprising (a) measuring the levels of antibodies that are reactive to at least three biomarkers selected from beta-2 microglobulin, C-reactive protein (CRP), and cystatin C and/or optionally measuring plasma glucose levels in a biological sample obtained from a subject and comparing the level of the antibodies of the least three biomarkers in the biological sample with a reference antibody level for each of beta-2 microglobulin, C-reactive protein (CRP) and cystatin C and/or reference plasma glucose level, and (b) determining the genotype of the allele at the rs10757269 loci; wherein a detectable increase of each antibody for each biomarker and/or increase of plasma glucose level in the biological sample obtained from the subject above the reference antibody level and/or reference plasma glucose level and/or where the genotyping assay indicates that the subject has at least one G-allele at the rs10757269 loci indicates the likelihood that the subject is at risk of having a major adverse event. In some embodiments, a subject at risk of a major cardiac event is selected for a treatment regimen and in some embodiments, a subject who is not at risk of a major cardiac event and/or does not have at least one G-allele at the rs10757269 loci is not treated with the treatment regimen. In some embodiments, appropriate treatment regimens administered to a subject at risk of a major cardiac event include, but are not limited to, an exercise program; control of blood pressure, decreased sugar intake, and/or decreased lipid levels, cessation of smoking, and administration of drug therapies including the administration of aspirin (with or without dipyridamole), clopidogrel, cilostazol, and/or pentoxifylline.

Another aspect of the present invention relates to methods, assays and systems to identify if a subject is at risk of PAD, comprising (a) measuring the levels of antibodies that are reactive to at least three biomarkers selected from beta-2 microglobulin, C-reactive protein (CRP), and cystatin C and/or optionally measuring plasma glucose levels in a biological sample obtained from a subject and comparing the level of the antibodies of the least three biomarkers in the biological sample with a reference antibody level for each of beta-2 microglobulin, C-reactive protein (CRP) and cystatin C and/or reference plasma glucose level, and (b) determining the genotype of the allele at the rs10757269 loci; wherein a detectable increase of each antibody for each biomarker and/or increase of plasma glucose level in the biological sample obtained from the subject above the reference antibody level and/or reference plasma glucose level and/or where the genotyping assay indicates that subject has at least one G-allele at the rs10757269 loci indicates the likelihood that the subject is at risk of having or developing PAD.

In some embodiments, the subject is a Caucasian subject, or a subject selected from the group consisting of, African-American, Hispanic or Asian subject, or an Asian-Indian, Pakistani, Middle Eastern or Pacific Islander ethnicity. In some embodiments, the subject is Caucasian, African-American or Asia-American.

In some embodiments, a treatment to prevent the occurrence a major adverse event is selected from the group of: an exercise program; control of blood pressure, reduced sugar intake, cessation of smoking and drug therapies selected from the group of aspirin (with or without dipyridamole), clopidogrel, cilostazol, and/or pentoxifylline.

In some embodiments, a major adverse event is stroke, heart attack or death, coronary bypass. In some embodiments, a major adverse event is a major adverse cardiovascular or cerebrovascular event (MACCE), for example, but not limited to recurrence of an initial cardiac event, angina, decompensation of heart failure, admission for cardiovascular disease (CVD), mortality due to CVD, myocardial infarction, stroke and transplant.

In some embodiments a threshold reference level for beta-2-microglobulin is 1.88 mg/l, and a threshold reference level for CRP is 1.60 mg/l, and a threshold reference level for cystatin C is 0.72 mg/l, and a threshold reference level for plasma glucose level is 99.96 mg/dL. Accordingly, in some embodiments, if a biological sample obtained from the subject has level of beta-2 microglobulin equal to, or above 1.88 mg/l, and has a level of CRP equal to, or above 1.60 mg/l, and has a level of cystatin C equal to or above 0.72 mg/l, and optionally has a level of plasma glucose equal to, or above, 99.96 mg/dL, the subject is diagnosed as having an increased risk of a major adverse event, and can optionally, be administered a therapy or treatment regimen to reduce the risk, or prevent the occurrence of a major adverse event.

In some embodiments a threshold reference level for plasma glucose is 99.96 mg/dL. Accordingly, in some embodiments, if a biological sample obtained from the subject has level of plasma glucose equal to, or above 99.96 mg/dL, the subject is diagnosed as having an increased risk of a major adverse event, and can optionally, be administered a therapy or treatment regimen to reduce the risk, or prevent the occurrence of a major adverse event.

In some embodiments, if a biological sample obtained from the subject has the presence of a G-allele at rs10757269, the subject is diagnosed as having an increased risk of a major adverse event and/or PAD, and can optionally, be administered a therapy or treatment regimen to reduce the risk, or prevent the occurrence of a major adverse event and/or PAD.

In some embodiments, a subject can be screened for either at least one or more of the biomarkers (e.g., level of beta-2-microglobulin, c-reactive protein (CRP) and cystatin C, and plasma glucose) or the presence of the G-allele at rs10757269. In some embodiments, a subject can be screened for the level of the biomarkers (e.g., for the level of one or more of beta-2-microglobulin, c-reactive protein (CRP) and cystatin C, and plasma glucose) and for the presence of the G-allele at rs10757269.

Another aspect of the present invention relates to an assay to determine if a subject is at risk of having a major adverse event, the assay comprising: (a) contacting a biological sample obtained from the subject with at least one probe to detect the levels of at least three biomarkers selected from beta-2 microglobulin, C-reactive protein (CRP) and cystatin C; (b) measuring the levels of at least three biomarkers selected from beta-2 microglobulin, C-reactive protein (CRP) and cystatin C; wherein the level of beta-2 microglobulin, C-reactive protein (CRP) and cystatin C above a threshold reference level for each of beta-2 microglobulin, C-reactive protein (CRP) and cystatin C identifies a subject who would be predicted to be at risk of having a major adverse event.

In some embodiments, a probe to detect the levels of the biomarkers comprises a detectable label or means of generating a detectable signal, and in some embodiments, the probe is an antibody, antibody binding fragment or protein binding molecule. In some embodiments, a probe can be bound to a solid support. In some embodiments, levels of the biomarkers as disclosed herein are measured using an immunoassay, e.g., immunoassay is an ELISA, or protein chip or the like.

In some embodiments, a level of beta-2-microglobulin equal to, or above 1.88 mg/l threshold reference level indicates that the subject is predicted to be at risk of having a major adverse event, and a level of CRP equal to, or above 1.60 mg/l threshold reference level indicates that the subject is predicted to be at risk of having a major adverse event, and a level of cystatin C equal to, or above 0.72 mg/l threshold reference level indicates that the subject is predicted to be at risk of having a major adverse event.

In some embodiments, the methods, assays and systems as disclosed herein can be used to determine if a subject is likely to have a major adverse event in the next 12 months or earlier.

In some embodiments, the methods, assay and systems can measure additional biomarkers, for example, biomarkers selected from the group consisting of, growth stimulation expressed gene 2 (ST2), natriuretic peptide (e.g., NT-proBMP)CD40, fibrinogen, IL-3, IL-8, SGOT and von Willebrand factor.

In some embodiments, the methods, assays and systems measure the levels of the biomarkers in a biological sample which is a blood-based sample or a urine sample, for example, where a blood based sample is a serum, plasma or blood sample. In some embodiments, a blood-based sample or urine sample is obtained from a human subject who has fasted.

In some embodiments, a subject is a human subject. In some embodiments, a subject has been diagnosed with heart failure, and/or has a body mass index (BMI) of 25 to 29, a BMI of greater or equal to 30. In some embodiments, a biological sample is obtained from a subject that has been hospitalized after an acute cardiac event. In some embodiments, a subject has a pulmonary disorder or a liver disorder, or is undergoing coronary angiography.

In some embodiments, the methods, assays and systems as disclosed herein can be used to assist a decision to discharge a subject or to continue treating a subject in an inpatient basis.

Another aspect of the present invention relates to a computer system for determining if a subject is at risk of having a major adverse event, the system comprising: (a) a measuring module configured to detect the levels of at least three biomarkers selected from beta-2 microglobulin, C-reactive protein (CRP) and cystatin C in a biological subject obtained from a subject; (b) a storage module configured to store output data from the measuring module; (c) a comparison module adapted to compare the data stored on the storage module with a reference threshold levels for beta-2 microglobulin, C-reactive protein (CRP) and cystatin, and to provide a retrieved content, and (d) a display module for displaying whether there the levels of beta-2 microglobulin, C-reactive protein (CRP) and cystatin C are at or above the reference threshold level, wherein the levels of beta-2 microglobulin, C-reactive protein (CRP) and cystatin C above the reference threshold level for each of beta-2 microglobulin, C-reactive protein (CRP) and cystatin C are above the reference threshold level indicate the subject is at risk of having a major adverse event, and/or displaying levels of beta-2 microglobulin, C-reactive protein (CRP) and cystatin C measured present in the biological sample. In some embodiments, if the comparison module determines that the levels of beta-2 microglobulin, C-reactive protein (CRP) and cystatin C in the biological sample obtained from the subject are at or above the reference threshold level, the display module displays a positive signal indicating that the subject is likely to be at risk of having a major adverse event, as compared to a subject who has levels of beta-2 microglobulin, C-reactive protein (CRP) and cystatin C below the reference threshold levels for beta-2 microglobulin, C-reactive protein (CRP) and cystatin C. In some embodiments, if the comparison module determines the levels of beta-2 microglobulin, C-reactive protein (CRP) and cystatin C in the biological sample obtained from the subject are below the reference threshold levels for beta-2 microglobulin, C-reactive protein (CRP) and cystatin C, the display module displays a negative signal indicating that the subject is not likely to be at risk of having a major adverse event, as compared to a subject who has levels of beta-2 microglobulin, C-reactive protein (CRP) and cystatin C at or above the reference threshold levels for beta-2 microglobulin, C-reactive protein (CRP) and cystatin C. In additional embodiments, the system can further comprise creating a report based on the levels of beta-2 microglobulin, C-reactive protein (CRP) and cystatin C in the biological sample obtained from the subject as compared to the reference threshold levels for beta-2 microglobulin, C-reactive protein (CRP) and cystatin C.

Also provided herein, in another aspect, are assays to select a subject at risk of having a major adverse event, the assay comprising: contacting a biological sample obtained from the subject with at least one probe to detect the levels of at least three biomarkers selected from beta-2 microglobulin, C-reactive protein (CRP) and cystatin C; measuring the levels of at least three biomarkers selected from beta-2 microglobulin, C-reactive protein (CRP) and cystatin C; wherein the level of beta-2 microglobulin, C-reactive protein (CRP) and cystatin C above a threshold reference level for each of beta-2 microglobulin, C-reactive protein (CRP) and cystatin C, thereby selecting a subject at risk of having a major adverse event.

Another aspect provided herein relates to an assay comprising: (a) measuring the levels of antibodies that are reactive to at least three biomarkers selected from beta-2 microglobulin, C-reactive protein (CRP), and cystatin C in a biological sample obtained from a subject who has a body mass index (BMI) of 25 or greater for determining the likelihood of the subject having a major adverse event; and (b) selecting a subject having an increased level of the antibodies of the least three biomarkers in the biological sample relative to a reference antibody level for each of beta-2 microglobulin, C-reactive protein (CRP) and cystatin C, as being at risk of having a major adverse event.

It is contemplated that any methods or compositions described herein can be implemented with respect of any other methods or compositions. Other objects, features and advantages will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1D shows cumulative all-cause mortality for beta-2 microglobulin, CRP and cystatin C. FIG. 1A shows the upper 50% of biomarker levels (red, equal or above 1.88 mg/L) as compared to the bottom 50% of biomarker levels (blue, below 1.88 mg/L) for Beta-2-microglobulin (median, 1.88 mg/L). FIG. 1B shows Cystatin C levels (median, 0.72 mg/L) and FIG. 1C shows C-reactive protein levels (median, 1.60 mg/L). FIG. 1D represents those individuals in the upper 50% of all three biomarkers (red) as compared to those individuals in the bottom 50% of all three biomarkers (blue).

FIGS. 2A-2D show represent cumulative cardiovascular mortality in the upper 50% of biomarker levels (red) as compared to the bottom 50% of biomarker levels (blue) for Beta-2-microglobulin (median, 1.88 mg/L), Cystatin C (median, 0.72 mg/L) and C-reactive protein (median, 1.60 mg/L). Frame D represents those individuals in the upper 50% of all three biomarkers (red) as compared to those individuals in the bottom 50% of all three biomarkers (blue).

FIG. 3 shows a simplified block diagram of an embodiment of the present invention which relates to a machine for determining the level of the biomarkers to predict a subject at risk of a major adverse event.

FIG. 4 of a machine 10 for determining the level of the biomarkers to predict a subject at risk of a major adverse event according to an embodiment of the invention.

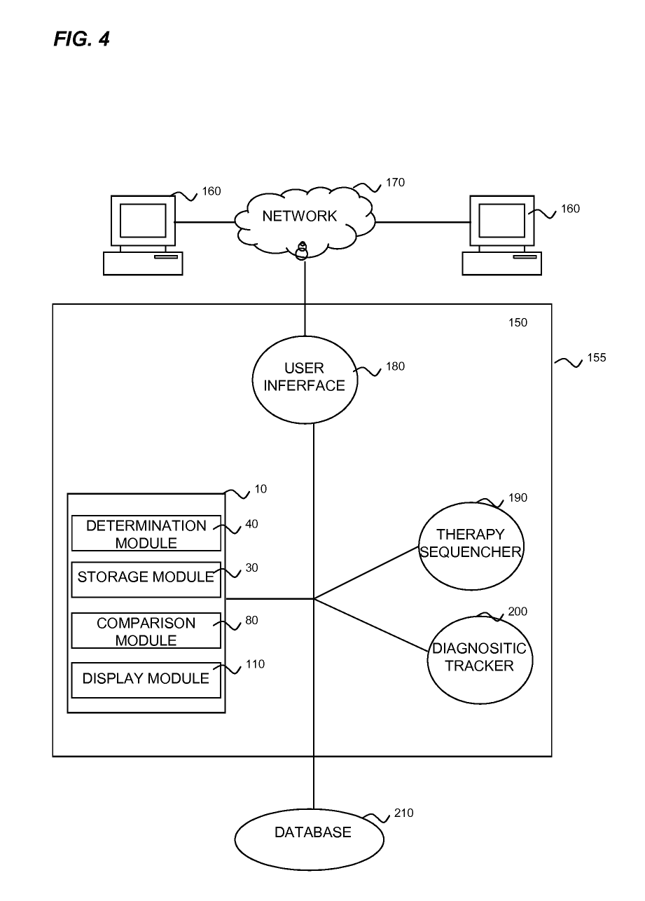

FIG. 5 depicts an exemplary block diagram of a computer system that may be configured to execute the prognostic application illustrated in FIG. 4.

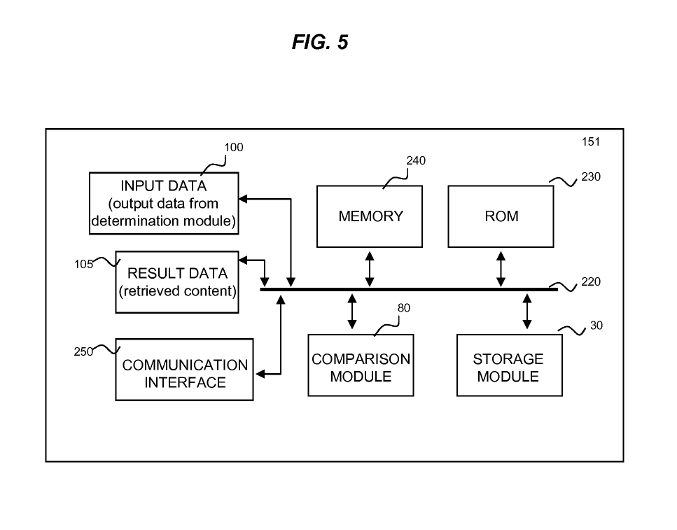

FIG. 6 shows a flow chart of instructions for analyzing if a subject is at risk of a major adverse event.

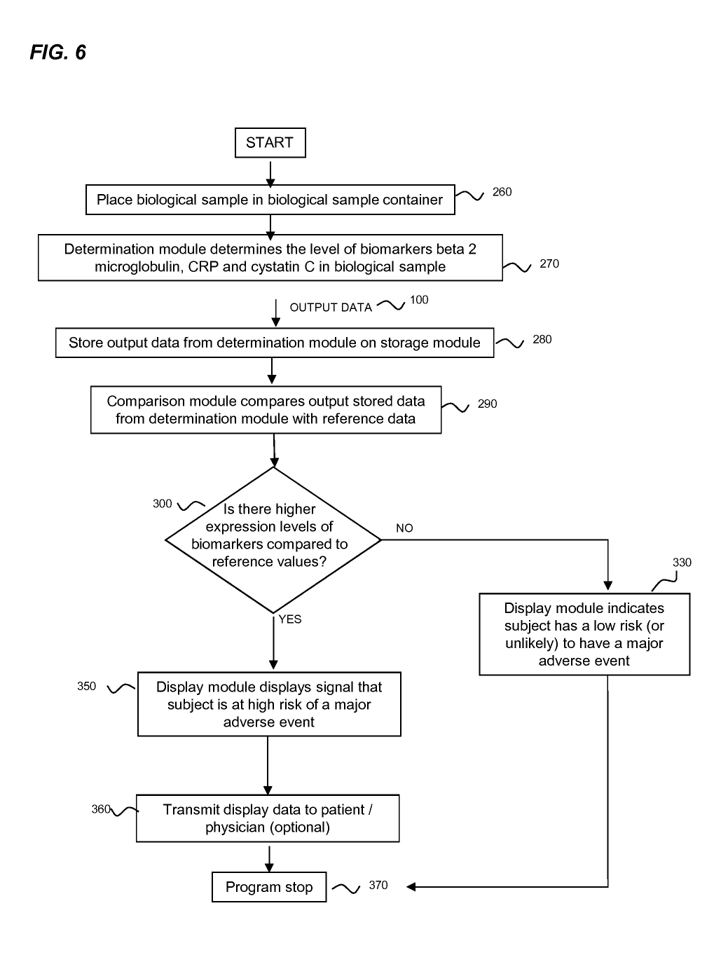

### DETAILED DESCRIPTION OF THE INVENTION

The present invention generally relates to diagnostic markers for predicting if a subject is at risk of a major adverse event. In particular, one aspect of the present invention relates to methods to determine if a subject is at risk of having a major adverse effect by measuring at least 2, or at least 3, of the biomarkers beta 2 microglobulin, c-reactive protein and cystatin C.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “biomarker” as used herein generally refers to an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median level of the biomarker in a first phenotypic status relative to a second phenotypic status is calculated to represent statistically significant differences. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. As such, biomarkers can find use as markers for, for example, disease (diagnostics), therapeutic effectiveness of a drug (theranostics), and of drug toxicity.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “higher” are all used herein to generally mean an increase by a statically significant amount; for the avoidance of any doubt, the terms “increased”, “increase” or “enhance” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

By an “increase” in the expression or activity of a gene or protein is meant a positive change in protein or polypeptide or nucleic acid level or activity in a cell, a cell extract, or a cell supernatant. For example, such an increase may be due to increased RNA stability, transcription, or translation, or decreased protein degradation. Preferably, this increase is at least 5%, at least about 10%, at least about 25%, at least about 50%, at least about 75%, at least about 80%, at least about 100%, at least about 200%, or even about 500% or more over the level of expression or activity under control conditions.

As used herein, the term “gene” includes a segment of DNA that contains all the information for the regulated biosynthesis of an RNA product, including promoters, exons, introns, and other untranslated regions that control expression. Those in the art will readily recognize that nucleic acid molecules can be double-stranded molecules and that reference to a particular site on one strand refers, as well, to the corresponding site on a complementary strand. Thus, in defining a polymorphic site, reference to an adenine, a thymine (uridine), a cytosine, or a guanine at a particular site on the plus (sense) strand of a nucleic acid molecule is also intended to include the thymine (uridine), adenine, guanine, or cytosine (respectively) at the corresponding site on a minus (antisense) strand of a complementary strand of a nucleic acid molecule. Thus, reference can be made to either strand and still comprise the same polymorphic site and an oligonucleotide can be designed to hybridize to either strand. Throughout this specification, in identifying a polymorphic site, reference is made to the sense strand, only for the purpose of convenience. As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytosine”, “guanosine”, and thymidine” are used. It is understood that if the nucleic acid is RNA, a nucleotide having a uracil base is uridine. The term “nucleotide” or nucleic acid as used herein is intended to refer to ribonucleotides, deoxyribonucleotides, acylic derivatives of nucleotides, and functional equivalents thereof, of any phosphorylation state. Functional equivalents of nucleotides are those that act as substrates for a polymerase as, for example, in an amplification method. Functional equivalents of nucleotides are also those that can be formed into a polynucleotide that retains the ability to hybridize in a sequence specific manner to a target polynucleotide. As used herein, the term “polynucleotide” includes nucleotides of any number. A polynucleotide includes a nucleic acid molecule of any number of nucleotides including single-stranded RNA, DNA or complements thereof, double-stranded DNA or RNA, and the like.

The term “sample” as used herein generally refers to any material containing nucleic acid, either DNA or RNA or amino acids. Generally, such material will be in the form of a blood sample, stool sample, tissue sample, cells, bacteria, histology section, or buccal swab. Samples can be prepared, for example samples can be fresh, fixed, frozen, or embedded in paraffin.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. A biological sample or tissue sample can refer to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples can be either paraffin-embedded or frozen tissue. The sample can be obtained by removing a sample of cells from a subject, but can also be accomplished by using previously isolated cells (e.g. isolated by another person), or by performing the methods of the present invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples can be fresh, fixed, frozen, or embedded in paraffin.

The term “expression” as used herein refers to the expression of a polypeptide or protein or expression of a polynucleotide or expression of a gene. Expression also refers to the expression of pre-translationally modified and post-translationally modified proteins, as well as expression of pre-mRNA molecules, alternatively spliced and mature mRNA molecules. Expression of a polynucleotide can be determined, for example, by measuring the production of RNA transcript molecules, for example messenger RNA (mRNA) transcript levels. Expression of a protein or polypeptide can be determined, for example, by immunoassay using an antibody(ies) that bind with the polypeptide.

The term “encode” as it is applied to polynucleotides refers to a polynucleotide which is said to “encode” a polypeptide or protein if, in its native state or when manipulated by methods well known to those skilled in the art, it can be transcribed to produce the RNA which can be translated into an amino acid sequence to generate the polypeptide and/or a fragment thereof. The antisense strand is the complement of such a nucleic acid, and the encoding sequence can be deduced therefrom.

The term “endogenously expressed” or “endogenous expression” refers to the expression of a gene product at normal levels and under normal regulation for that cell type.

As used herein, the terms “isoform”, or “isoforms” or “variant of protein” are used interchangeably herein, refer to specific forms of the same protein, the specific form differing from other forms of the same protein in the sequence of at least one, and frequently more than one, amino acid(s). Isoforms are proteins produced from the same gene due to, for example but not limited to, transcription from different promoters, alternative splicing, differential mRNA splicing and/or post-translational modification such as, for example, glycosylation, sumoylation, phosphorylation, truncation and ectodomain shedding.

The term “primer”, as used herein, refers to an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature, A primer must be sufficiently long to prime the synthesis of extension products in the presence of an agent for polymerase. A typical primer contains at least about 5 nucleotides in length of a sequence substantially complementary to the target sequence, but somewhat longer primers are preferred. Usually primers contain about 15-26 nucleotides, but longer primers can also be employed. Oligonucleotides, such as “primer” oligonucleotides are preferably single stranded, but can alternatively be double stranded. If double stranded, the oligonucleotide is generally first treated to separate its strands before being used for hybridization purposes or being used to prepare extension products. Primer oligonucleotides can be oligodeoxyribonucleotide. A primer will always contain a sequence substantially complementary to the target sequence which is the specific sequence to be amplified, to which it can anneal, A primer may, optionally, also comprise a promoter sequence.

In the context of this invention, the term “probe” refers to a molecule which can detectably distinguish between target molecules differing in structure. Detection can be accomplished in a variety of different ways depending on the type of probe used and the type of target molecule, thus, for example, detection can be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and preferably nucleic acid hybridization probes, for example DNA, RNA, PNA, pseudo-complementary PNA (pcPNA), locked nucleic acid (LNA) and nucleic acid analogues thereof.

Oligonucleotides can be used as “probes”, and refer to e.g., genomic DNA, mRNA, or other suitable sources of nucleic acid oligonucleotides. For such purposes, the oligonucleotides must be capable of specifically hybridizing to a target polynucleotide or DNA nucleic acid molecule. As used herein, two nucleic acid molecules are said to be capable of specifically hybridizing to one another if the two molecules are capable of forming an anti-parallel, double-stranded nucleic acid structure under hybridizing conditions.

The term “allele-specific oligonucleotide” refers to an oligonucleotide that is able to hybridize to a region of a target polynucleotide spanning the sequence, mutation, or polymorphism being detected and is substantially unable to hybridize to a corresponding region of a target polynucleotide that either does not contain the sequence, mutation, or polymorphism being detected or contains an altered sequence, mutation, or polymorphism. As will be appreciated by those in the art, allele-specific is not meant to denote an absolute condition. Allele-specificity will depend upon a variety of environmental conditions, including salt and formamide concentrations, hybridization and washing conditions and stringency. Depending on the sequences being analyzed, one or more allele-specific oligonucleotides can be employed for each target polynucleotide. Preferably, allele-specific oligonucleotides will be completely complementary to the target polynucleotide. However, departures from complete complementarity are permissible. In order for an oligonucleotide to serve as a primer oligonucleotide, however, it typically need only be sufficiently complementary in sequence to be able to form a stable double-stranded structure under the particular environmental conditions employed. Establishing environmental conditions typically involves selection of solvent and salt concentration, incubation temperatures, and incubation times.

The term “hybridizing” as used herein, refers to the binding of one nucleic acid sequence to another by complementation or complementary base pair matching.

A nucleic acid molecule is said to be the “complement” of another nucleic acid molecule if it exhibits complete complementarity. As used herein, molecules are said to exhibit “complete complementarity” when every nucleotide of one of the molecules is complementary to a nucleotide of the other. Two molecules are said to be “substantially complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described, for example, by Sambrook, J., et al, in Molecular Cloning, a Laboratory Manual, 2nd Edition, Cold Spring Harbor Press, Cold Spring Harbor, N.Y. (1989), and by Haymes, B. D., et al. in Nucleic Acid Hybridization, A Practical Approach, IRL Press, Washington, D.C. (1985), both herein incorporated by reference). Departures from complete complementarity are therefore permissible, as long as such departures do not completely preclude the capacity of the molecules to form a double-stranded structure. For example, a non-complementary nucleotide fragment can be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith for the purposes employed. However, for detection purposes, particularly using labeled sequence-specific probes, the primers typically have exact complementarity to obtain the best results. Thus, for an oligonucleotide to serve as an allele-specific oligonucleotide, it must generally be complementary in sequence and be able to form a stable double-stranded structure with a target polynucleotide under the particular environmental conditions employed.

The term “real-time quantitative RT-PCR” or “quantitative RT-PCR” or “QRT-PCR” are used interchangeably herein, refers to reverse transcription (RT) polymerase chain reaction (PCR) which enables detection of gene transcription. The method is known to those ordinary skilled in the art and comprises of the reverse transcription and amplification of messenger RNA (mRNA) species to cDNA, which is further amplified by the PCR reaction. QRT-PCR enables a one skilled in the art to quantitatively measure the level of gene transcription from the test gene in a particular biological sample. The methods of RNA isolation, RNA reverse transcription (RT) to cDNA (copy DNA) and cDNA or nucleic acid amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Möller, R. Beynon, C. Howe, Springer Verlag; 1st edition (Oct. 15, 2000), ISBN: 0387916008.

The term “multiplex” as used herein refers to the testing and/or the assessment of more than one gene within the same reaction sample.

The term “amplify” is used in the broad sense to mean creating an amplification product which can include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases. The term “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR).

The term “Homology” or “identity” or “similarity” refers to sequence similarity between two peptides or between two nucleic acid molecules. Homology can be determined by comparing a position in each sequence which can be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than about 40% identity, though preferably less than about 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, e. hybridization assay. The term interact is also meant to include “binding” interactions between molecules. Interactions can be, for example, protein-protein, protein-nucleic acid, protein-small molecule or small molecule-nucleic acid in nature.

The term “isolated” as used herein with respect to nucleic acids, such as DNA or RNA, refers to molecules separated from other DNAs or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to, polypeptides which are isolated from other cellular proteins and is meant to encompass both purified and recombinant polypeptides.

The term “mismatches” refers to hybridized nucleic acid duplexes which are not 100% homologous. The lack of total homology can be due to deletions, insertions, inversions, substitutions or frame shift mutations.

As used herein, the terms “effective” and “effectiveness” or “responsive” includes both pharmacological effectiveness and physiological safety of an agent. “Pharmacological effectiveness” refers to the ability of the treatment to result in a desired biological effect in the subject. “Physiological safety” refers to the level of toxicity, or other adverse physiological effects at the cellular, organ and/or organism level (often referred to as side-effects) resulting from administration of the treatment, “less effective” means that the treatment results in a therapeutically significant lower level of pharmacological effectiveness and/or a therapeutically greater level of adverse physiological effects.

The term “lack of effectiveness”, “non-responsiveness”, “refractory” or “unresponsiveness” are used interchangeably herein, and refer to the inability of an agent or treatment to result in a desired biological effect in the subject.

The term “activity” when used in reference to the activity of a protein as used herein, comprises the enzymatic activity, binding affinity and/or posttranslational activity, in particular phosphorylation.

The term “target” as used herein may mean a polynucleotide that may be bound by one or more probes under stringent hybridization conditions.

The term “entity” refers to any structural molecule or combination of molecules.

The term “drug”, “agent” or “compound” as used herein refers to a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “agent” refers to any entity, which is normally absent or not present at the levels being administered, in the cell. Agent may be selected from a group comprising; chemicals; small molecules; nucleic acid sequences; nucleic acid analogues; proteins; peptides; aptamers; antibodies; or fragments thereof. A nucleic acid sequence may be RNA or DNA, and may be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The term “antagonist” refers to any agent or entity capable of inhibiting the expression or activity of a protein, polypeptide portion thereof, or polynucleotide. Thus, the antagonist may operate to prevent transcription, translation, post-transcriptional or post-translational processing or otherwise inhibit the activity of the protein, polypeptide or polynucleotide in any way, via either direct or indirect action. The antagonist may for example be a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide or polynucleotide itself. Similarly, the antagonist may affect the activity of molecules which are themselves subject to the regulation or modulation by the protein, polypeptide of polynucleotide.

The term “inhibiting” as used herein as it pertains to the expression or activity of the protein or polypeptide of topoisomerase I or variants thereof does not necessarily mean complete inhibition of expression and/or activity. Rather, expression or activity of the protein, polypeptide or polynucleotide is inhibited to an extent, and/or for a time, sufficient to produce the desired effect.

The term “protein binding moiety” is used interchangeably herein with “protein binding molecule” or protein binding entity” and refers to any entity which has specific affinity for a protein. The term “protein-binding molecule” also includes antibody-based binding moieties and antibodies and includes immunoglobulin molecules and immunologically active determinants of immunoglobulin molecules, e.g., molecules that contain an antigen binding site which specifically binds (immunoreacts with) to a biomarker protein. The term “antibody-based binding moiety” is intended to include whole antibodies, e.g., of any isotype (IgG, IgA, IgM, IgE, etc), and includes fragments thereof which are also specifically reactive with the Psap proteins. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's can be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In a preferred embodiment, the antibody-based binding moiety detectably labeled. In some embodiments, a “protein-binding molecule” is a co-factor or binding protein that interacts with the protein to be measured, for example a co-factor or binding protein to a biomarker protein.

The term “antibody” is meant to include any of a variety of forms of antibodies that specifically bind an antigen of interest, including complete antibodies, fragments thereof (e.g., F(ab′)2, Fab, etc.), modified antibodies produced therefrom (e.g., antibodies modified through chemical, biochemical, or recombinant DNA methodologies), single chain antibodies, and the like, with the proviso that the antibody fragments and modified antibodies retain antigen binding characteristics sufficient to facilitate specific detection of an antigen of interest (e.g., B2M, or CRP or cystain-c) in an immunoassay. The term “antibody” is meant to be an immunoglobulin protein that is capable of binding an antigen. Antibody as used herein is meant to include antibody fragments, e.g. F(ab′)₂, Fab′, Fab, capable of binding the antigen or antigenic fragment of interest.

The term “labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS. The detection and quantification of biomarkers present in the tissue samples correlate to the intensity of the signal emitted from the detectably labeled antibody.

The term “specific affinity” or “specifically binds” or “specific binding” are used interchangeably herein refers to an entity such as a protein-binding molecule or antibody that recognizes and binds a desired polypeptide but that does not substantially recognize and bind other molecules in a sample, for example, a biological sample, which naturally includes a polypeptide of the invention, for example a biomarker selected from beta-2 microglobulin, CRP or cystatin C.

The term “specifically binds”, “specifically immunologically cross reactive with,” or “specifically immunoreactive with” when referring to a protein or a binding partner that binds a protein (e.g., an antibody), refers to a binding reaction between a protein and a binding partner (e.g., antibody) which is determinative of the presence of the protein in the presence of a heterogeneous population of proteins and other biologics. Thus, under designated conditions, a specified binding partner (e.g., antibody) binds preferentially to a particular protein and does not bind in a significant amount to other proteins present in the sample. A binding partner (e.g., an antibody) that specifically binds to a protein has an association constant of at least 10³ M⁻¹ or 10⁴ M⁻¹, sometimes 10⁵ M⁻¹ or 10⁶ M⁻¹, in other instances at least 10⁶ M⁻¹ or 10⁷ M⁻¹, or at least 10⁸ M⁻¹ to 10⁹ M⁻¹, or at least 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

The term “humanized antibody” is used herein to describe complete antibody molecules, i.e. composed of two complete light chains and two complete heavy chains, as well as antibodies consisting only of antibody fragments, e.g. Fab, Fab′, F(ab′)₂, and Fv, wherein the CDRs are derived from a non-human source and the remaining portion of the Ig molecule or fragment thereof is derived from a human antibody, preferably produced from a nucleic acid sequence encoding a human antibody.

The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human subject.

The term “chimeric antibody” is used herein to describe an antibody molecule as well as antibody fragments, as described above in the definition of the term “humanized antibody.” The term “chimeric antibody” encompasses humanized antibodies Chimeric antibodies have at least one portion of a heavy or light chain amino acid sequence derived from a first mammalian species and another portion of the heavy or light chain amino acid sequence derived from a second, different mammalian species. In some embodiments, a variable region is derived from a non-human mammalian species and the constant region is derived from a human species. Specifically, the chimeric antibody is preferably produced from a 9 nucleotide sequence from a non-human mammal encoding a variable region and a nucleotide sequence from a human encoding a constant region of an antibody.

In the context of this invention, the term “probe” refers to a molecule which can detectably distinguish between target molecules differing in structure. Detection can be accomplished in a variety of different ways depending on the type of probe used and the type of target molecule, thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and preferably nucleic acid hybridization probes.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

The term “tissue” is intended to include intact cells, blood, blood preparations such as plasma and serum, bones, joints, muscles, smooth muscles, and organs.

The terms “patient”, “subject” and “individual” are used interchangeably herein, and refer to an animal, particularly a human, to whom treatment including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The terms “individual,” “subject,” “host,” and “patient,” used interchangeably herein, refer to a mammal, including, but not limited to, murines, simians, humans, felines, canines, equines, bovines, mammalian farm animals, mammalian sport animals, and mammalian pets. Human subjects are of particular interest.

The term “biological sample” as used herein refers to a sample obtained from blood of a subject for analysis of B2M and/or CRP levels and cystatin C, and includes a clinical sample, as well as samples that have been stored (with the proviso that storage under conditions to avoid degradation of B2M and CRP and cystatin C). Exemplary biological samples of blood include peripheral blood or samples derived from peripheral blood. In some cases, the blood will have been enriched for a protein fraction containing B2M and/or CRP.

The term “blood sample” or “blood-based sample” as used herein refers to a sample which is derived from blood, usually peripheral (or circulating) blood. A blood sample may be, for example, whole blood, plasma or serum.

As used herein, the terms “treatment” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease. In some embodiments, the term “treating” includes reducing or alleviating at least one adverse effect or symptom of a condition, disease or disorder associated with cancer. As used herein, the term treating is used to refer to the reduction of a symptom and/or a biochemical marker of a major adverse event, for example a reduction in at least one biochemical marker (e.g., beta-2 microglobulin, CRP or cystatin C) of a major adverse event by at least 10%.

The term “polynucleotide” as used herein, refers to single- or double-stranded polymer of deoxyribonucleotide, ribonucleotide bases or known analogies of natural nucleotides, or mixtures thereof. The term includes reference to the specified sequence as well as to the sequence complementary thereto, unless otherwise indicated.

The term “polypeptide” means a polymer made up of amino acids linked together by peptide bonds. The terms “polypeptide” and “protein” are used interchangeably herein, although for the purposes for the present invention, a polypeptide may constitute a portion or the full length protein.

The term “expression” as used herein refers to interchangeably to the expression of a polypeptide or protein and expression of a polynucleotide or gene. Expression of a polynucleotide may be determined, for example, by measuring the production of messenger RNA (mRNA) transcript levels. Expression of a protein or polypeptide may be determined, for example, by immunoassay using an antibody(ies) that bind with the polypeptide.

The term “endogenously expressed” or “endogenous expression” as used herein, refers to the expression of a gene product at normal levels and under normal regulation for that cell type.

In the context of this specification, the term “activity” as it pertains to a protein, polypeptide or polynucleotide means any cellular function, action, effect of influence exerted by the protein, polypeptide or polynucleotide, either by nucleic acid sequence or fragment thereof, or by the protein or polypeptide itself or any fragment thereof.

The term “nucleic acid” or “oligonucleotide” or “polynucleotide” used herein can mean at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions.

The term “computer” can refer to any apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” can refer to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “proteomics” may refer to the study of the expression, structure, and function of proteins within cells, including the way they work and interact with each other, providing different information than genomic analysis of gene expression.

As used herein, the terms “determining”, “assessing”, “assaying”, “measuring” and “detecting” refer to both quantitative and qualitative determinations and as such, the term “determining” is used interchangeably herein with “assaying,” “measuring,” and the like. Where a quantitative determination is intended, the phrase “determining an amount” of an analyte and the like is used. Where either a qualitative or quantitative determination is intended, the phrase “determining a level” of an analyte or “detecting” an analyte is used

Compositions or methods “comprising” one or more recited elements may include other elements not specifically recited. For example, a composition that comprises a fibril component peptide encompasses both the isolated peptide and the peptide as a component of a larger polypeptide sequence. By way of further example, a composition that comprises elements A and B also encompasses a composition consisting of A, B and C. The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.” In the context of the specification, the term “comprising” means “including principally, but not necessary solely”. Furthermore, variation of the word “comprising”, such as “comprise” and “comprises”, have correspondingly varied meanings.

The use of the word “a” or “an” when used in conjunction with the term “comprising” in the claims and/or the specification may mean “one” but is also consistent with the meaning of “one or more”, “at least one” and “one or more than one.”

Other than in the operating examples, or where otherwise indicated, all numbers expressing quantities of ingredients or reaction conditions used herein should be understood as modified in all instances by the term “about.” The term “about” when used in connection with percentages can mean±1%.

This invention is further illustrated by the following examples which should not be construed as limiting. The contents of all references cited throughout this application, as well as the figures and tables are incorporated herein by reference.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Methods to Identify if a Subject is at Risk of a Major Adverse Event

The present invention features diagnostic and prognostic methods, which are based in part, on the detection of the levels of 3 biomarkers; beta 2 microglobulin, C-reactive peptide (CRP) and cystatin C in a biological sample, and if the levels of the three biomarkers are elevated above a reference value for each biomarker, the subject is identified to have an increased risk or high risk of having a major adverse event, such as a stroke, heart attack or death.

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a-subject belongs to one phenotypic status or another. As such, they are useful as markers for disease (diagnostics), therapeutic effectiveness of a drug (theranostics) and of drug toxicity.

### β-2 Microglobulin

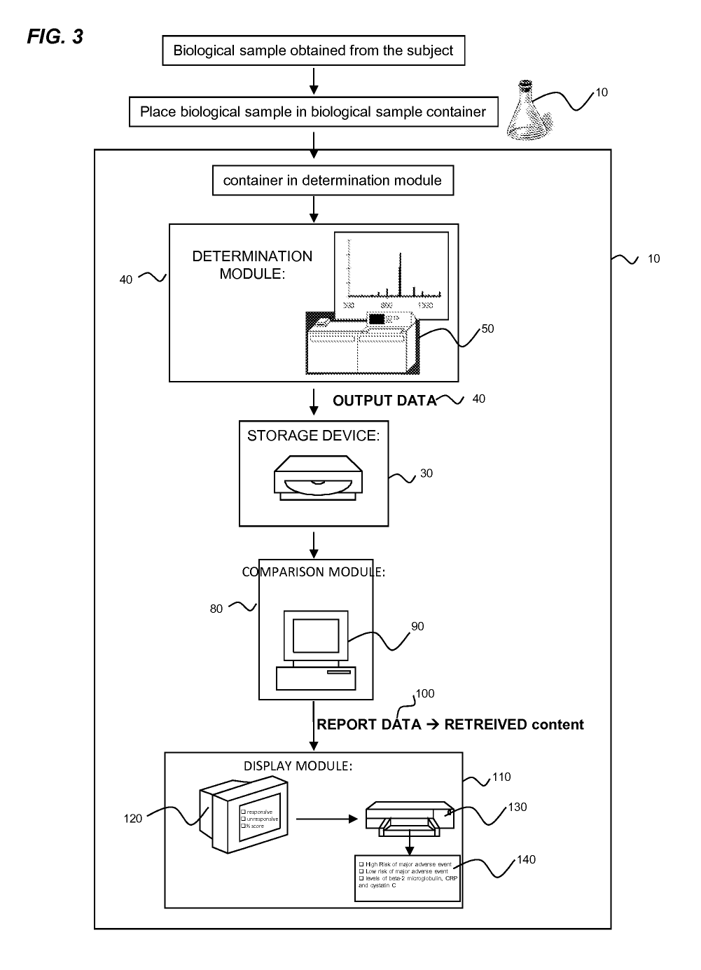

Useful protein biomarkers for detecting a subject at risk of a major adverse event include beta-2-microglobulin, Cystatin C, and CRP. The inventors have discovered that beta2-microglobulin is useful as a biomarker, combined with other biomarkers such as CRP, cystatin-C to identify a subject with a high risk of a major adverse event. The mass of beta2-microglobulin corresponds to a 11.7K Dalton biomarker, which is described as a biomarker for peripheral artery disease in International Patent Publication WO 2005/121758, and U.S. Pat. Nos. 7,998,743; 8,053,204; 8,227,201; 8,008,020; and 8,090,562 and US application 2011/0045514 which are all incorporated herein in their entirety by reference. Beta 2-microglobulin is a 99 amino acid protein derived from a 119 amino acid precursor (GI:179318; SwissProt Accession No. P61769). Beta 2-microglobulin is recognized by antibodies available from, e.g., ABCAM™ (catalog AB759) (Cambridge, Mass.). Specifics of the beta 2-microglobulin biomarker are presented in Table 1, Table 2 and FIG. 3 of U.S. Pat. No. 8,053,204, which is incorporated herein in its entirety by reference.

Beta-2-microglobulin is a 99 amino acid protein derived from a 119 amino acid precursor (GI:179318; SwissProt Accession No. P61769) and is recognized by antibodies available from, e.g., ABCAM™ (catalog AB759) (Cambridge, Mass.). Levels of beta 2-microglobulin less than 1.85 mg/ml are considered within normal limits.

A QHyperDF column can be used to purify the biomarkers (e.g., beta-2-microglobulin, Cystatin C, and CRP) from plasma, as described in, e.g., U.S. patent application Ser. No. 11/685,146 and U.S. Pat. No. 7,998,743, which are incorporated herein in their entirety by reference. IMAC-Cu⁺⁺ and CM10 refer to commercially available Proteinchips comprising metal chelating and cation exchange adsorbents, respectively. The biomarkers can elute in different fractions from a QHyper DF column (BioSepra, Cergy, France), as disclosed in the last column of Table 1 of U.S. Pat. No. 7,998,743. Antibodies that specifically bind B2M can be generated using methods known in the art. In addition, antibodies that specifically bind B2M are available from commercial sources. Examples of commercially available anit-B2M antibodies include, without limitation, antibodies available from, e.g., ABCAM™ (catalog AB759) (Cambridge, Mass.).

In the context of a biomarker panel useful for diagnosing and/or assessing risk of a major adverse event, other metabolites (e.g., glucose) may be measured, as well as other protein biomarkers in addition to beta-2-microglobulin (B2M).

Herein, the range of beta-2-microglobulin was between 1.50-2.57 mg/l (see Table 1 in the Examples). In some embodiments, a human subject with a beta-2-microglobulin blood or serum level at or above a reference threshold level of 1.88 mg/l indicates that the subject is at risk of having a major adverse event.

### C-Reactive Protein (CRP)

C-reactive protein (herein referred to also as “CRP,” or “hsCRP” for “high sensitivity CRP”) is a homopentameric oligoprotein composed of monomeric subunits that are each about 21 kD. The human CRP molecule has a relative molecular weight of about 115 kDa (115,135 Da), and is composed of five identical non-glycosylated polypeptide subunits, each having a relative molecular weight of about 23 kDa (23,027 Da), and each containing 206 amino acid residues (Hirschfield and Pepys Q J Med 2003; 96: 793-807). The form of CRP detected in the assays of the present disclosure is usually the pentameric form, particularly where the assay detects CRP based on molecular weight. Serum levels of hsCRP are elevated in individuals at risk for peripheral artery disease. Based upon the published literature, the American Heart Association recommends that hsCRP be used to “detect enhanced absolute risk in persons in whom multiple risk factor scoring (based on the Framingham Heart Study global risk scoring system) projects a 10-year CHD risk in the range of 10% to 20%.” In some embodiments, CRP can be used to determine those at lower or greater risk. Risk would be relatively “low” with CRP levels of less than 1 mg/L; “average” at 1-3 mg/L; and “high” at levels greater than 3 mg/L. Herein, the range of CRP was between 0.60-4.30 mg/l (see Table 1 in the Examples). In some embodiments, a human subject with a CRP blood or serum level at or above a reference threshold level of 1.60 mg/l indicates that the subject is at risk of having a major adverse event.

CRP preferentially binds to phosphorylcholine, a common constituent of microbial membranes. The interaction of CRP with phosphorylcholine promotes agglutination and opsonization of bacteria, as well as activation of the complement cascade, all of which are involved in bacterial clearance. CRP can also interact with DNA and histones. The normal plasma concentration of CRP is less than about 3 m/ml (30 nM) in 90% of the healthy population, and less than about 10 μg/ml (100 nM) in 99% of healthy individuals. It will be appreciated that normal values may exhibit variation in accordance with certain population characteristics such as race, ethnicity, gender, and the like.

Antibodies that specifically bind CRP can be generated using methods known in the art. In addition, antibodies that specifically bind CRP, including monoclonal anti-CRP antibodies, are available from commercial sources. Examples of commercially available anti-CRP antibodies include, without limitation, antibodies available from, e.g., ABCAM™ (catalog AB8280) (Cambridge, Mass.). In addition, one skilled in the art would know how to generate or obtain antibodies for the purpose of measuring CRP in human serum.

### Cystatin C

Cystatin C (sometimes referred to as cystatin 3) is a cysteine protease inhibitor found in serum that is sometimes used as a biomarker for kidney function. Antibodies useful for detecting cystatin C are readily available. The range of Cystatin C in human serum is between 0.5 and 0.99 mg/dl (see, e.g., Uhlmann E J et al., Clin Chem. 2001; 47(11):2031-2033). Herein, the range of cystatin C was between 0.61-0.93 mg/l (see Table 1 in the Examples). In some embodiments, a human subject with a cystatin C blood or serum level at or above a reference threshold level of 0.72 mg/l indicates that the subject is at risk of having a major adverse event.

In one embodiment of a biomarker panel for diagnosing PAD, the protein biomarkers cystatin C, hsCRP and/or beta 2-microglobulin levels in serum are measured in addition to glucose levels. Methods for measuring glucose levels in humans are well-known in the art. Blood glucose is typically measured after fasting (e.g., collected after an 8 to 10 hour fast), and/or as part of an oral glucose tolerance test (OGTT/GTT). Normal fasting levels of glucose are below 100 mg/dl.

Other protein biomarkers such as hemoglobin A1c and/or glycated hemoglobin whose levels may be correlated with glucose levels can also be measured and used in the context of the biomarker panel for a major adverse event as described herein. Healthy persons typically have levels of hemoglobin A1c from 4-5.9%. Because higher levels of hemoglobin A1c are associated with higher levels of blood glucose (see, e.g., Koenig R J et al. (1976) N. Engl. J. Med. 295 (8):417-20; Larsen et al. (1990). N. Engl. J. Med. 323 (15):1021-5), the detection of higher levels of hemoglobin A1c is a useful indicator of increased risk of PAD in a subject according to the diagnostic methods described herein. A variety of kits and methods for the detection of A1c are available and well-known to those of ordinary skill in the art.

### Detection of Biomarkers Beta2 Microglobulin, CRP and Cystatin-C

The beta2-microglobulin, cystatin C and CRP biomarkers of the present invention can be detected by any suitable method, including detection or protein levels or detection of mRNA expression levels. B2M, CRP and cystatin C polypeptides can be detected in any form that may be found in a biological sample obtained from a subject, or in any form that may result from manipulation of the biological sample (e.g., as a result of sample processing). Modified forms of B2M, CRP and/or cystatin C can include modified proteins that are a product of allelic variants, splice variants, post-translational modification (e.g., glycosylation, proteolytic cleavage (e.g., fragments of a parent protein), glycosylation, phosphorylation, lipidation, oxidation, methylation, cysteinylation, sulphonation, acetylation, and the like), oligomerization, de-oligomerization (to separate monomers from a multimeric form of the protein), denaturation, and the like.

The assays described herein can be designed to detect all forms or particular forms of either B2M, CRP and cystain c. Where desired, differentiation between different forms of the same protein can be accomplished by use of detection methods dependent upon physical characteristics that differ between the forms, e.g., different molecular weight, different molecular size, presence/absence of different epitopes, and the like.

Detection paradigms include optical methods, electrochemical methods (e.g., voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

One aspect of the present invention provides a method for the diagnosis of a subject at risk of a major adverse event, the method comprising measuring the level of at least three biomarker proteins, e.g., beta-2-microglobulin, CRP and cystatin C proteins in a biological sample obtained from the subject, wherein if the level of the biomarker proteins, e.g., beta-2-microglobulin, CRP and cystatin C in the biological sample from the subject are at the same level or greater than (e.g., greater than by a statistically significant amount) the threshold reference levels for the biomarker proteins, e.g., beta-2-microglobulin, CRP and cystatin C protein, the subject likely is at risk of having a major adverse event. For example, if the levels of biomarker proteins, e.g., beta-2-microglobulin, CRP and cystatin C measured in the subject are at or above 1.88 mg/l, 1.60 mg/l and 0.72 mg/l for beta-2-microglobulin, CRP and cycstain C, respectively, the subject is identified to be at risk of a major adverse event.

In some embodiments, the greater increase from the reference threshold level of biomarker indicates the higher risk of having a major adverse event. For example, a subject who has blood levels of biomarker proteins, e.g., beta-2-microglobulin, CRP and cystatin C that are 50% greater than the reference threshold levels for each of beta-2-microglobulin, CRP and cystatin C will be at a higher risk for a major adverse event as compared to a subject who has blood levels of the biomarkers that are only 10% higher than the reference threshold levels for each biomarker.

Accordingly, one aspect of the present invention relates to a method for assessing a subject at risk of having a major adverse event, for example, at risk for a major adverse cardiac event (MACE), the method comprising measuring the level of biomarker proteins, e.g., beta-2-microglobulin, CRP and cystatin C in a biological sample obtained from the subject, wherein an increase in the level of biomarker proteins, e.g., beta-2-microglobulin, CRP and cystatin C in the biological sample by a statistically significant amount as compared to a threshold reference level for each biomarker protein is indicative of the subject being at risk of having a major adverse event. In some embodiments, an increase in the level of a biomarker protein, e.g., beta-2-microglobulin, CRP and cystatin C in the biological sample by more than about 10%, or more than about 20%, or more than about 30%, or more than about 40%, or more than about 50%, or more than about 60%, as compared to a reference threshold level of biomarker proteins, e.g., beta-2-microglobulin, CRP and cystatin C is indicative of the subject being at risk of having a major adverse event.

In some embodiments, the amount of biomarker protein, e.g., beta-2-microglobulin, CRP and cystatin C measured in a biological sample is compared to a reference threshold level, or a reference biological sample, such as biological sample obtained from an age-matched normal control (e.g. an age-matched subject not having a risk of an adverse event), or a healthy subject, e.g., a healthy individual. In some embodiments, a reference threshold level or value of the biomarker protein is as follows: the threshold reference level for blood levels of beta-2-microglobulin is 1.88 mg/l, the reference threshold level for blood levels of CRP is 1.60 mg/l and the reference threshold level for blood levels of cystatin C is 0.72 mg/l. Thus, if there is a statistically significant decrease in a biomarker protein, e.g., beta-2-microglobulin, CRP and cystatin C in a serum sample from a subject that is at or above 1.88 mg/l, or 1.60 mg/l or 0.72 mg/l for biomarker proteins beta-2-microglobulin, CRP and cystatin C, respectively, then the subject is at risk of having a major adverse event. Thus, if a test subject has at least a 1% more, or at least about a 10% more, or at least a 20% more, or at least about a 30% more or at least about 40% more or at least about 50% more or greater than 50% of the level of the reference threshold level of each biomarker protein, (e.g., beta-2-microglobulin, CRP and cystatin C), then the subject likely to be at risk of having a major adverse event.

Stated another way, if the measured level of the panel of biomarker proteins, e.g., beta-2-microglobulin, CRP and cystatin C in the biological sample from the subject is the same or higher (e.g., increased) by a statistically significant amount as compared to the reference threshold level for each biomarker, then it is indicative of the subject being at risk of having a major adverse event.

In some embodiments, the methods, systems and kits as disclosed herein also are useful for monitoring a course of treatment being administered to a subject. For example, one can measure the level of the panel of biomarker proteins, (e.g., beta-2-microglobulin, CRP and cystatin C) in a biological sample in the subject at a first timepoint (e.g., t1) and compare with each biomarker reference threshold level, and if the measured level for each biomarker in the panel is the same or higher than the reference threshold level, the subject can be administered an appropriate therapeutic treatment or regimen to reduce the occurrence of a major adverse event, e.g., for example, increase exercise, reduce heart pressure, reduced caloric intake, diet modifications etc. as disclosed in the methods herein, and then the level of the panel of biomarker proteins, (e.g., beta-2-microglobulin, CRP and cystatin C) can be measured at a second (e.g., t2) and subsequent timepoints (e.g., t3, t4, t5, t5 . . . etc.), and compared to levels of the panel of biomarker proteins, (e.g., beta-2-microglobulin, CRP and cystatin C) at one or more time points (e.g., at t1 or any subsequent timepoint) or the reference threshold levels of each biomarker to determine if a therapeutic treatment or medical treatment or regimen for the treatment to reduce the risk of a major adverse event is effective. In some embodiments, the methods, systems and kits as disclosed herein can be used to monitor a therapeutic treatment in symptomatic subject (e.g., a subject with a risk of a major adverse event) where an effective treatment can be a decrease in one or more of the biomarkers in the panel of biomarker proteins (e.g., beta-2-microglobulin, CRP and cystatin C) in the subject, or alternatively the methods, systems and kits as disclosed herein can be used to monitor the effect of prophylactic treatment in asymptomatic subject (e.g., to prevent a major adverse event occurring in a subject), for example, where the subject has been identified to be at risk of a major adverse event according to the methods as disclosed herein.

### Biological Sample

In some embodiments, a biological sample for use in the methods and systems as disclosed herein is a peripheral biological fluid sample, for example, any one of the samples selected from: blood, plasma, serum, urine, mucus or cerebral spinal fluid obtained from the subject. A biological sample can be taken from any biological sample, e.g. a valid body tissue, especially body fluid, of a (human) subject, but preferably blood, plasma or serum. Other usable body fluids include cerebrospinal fluid (CSF), urine and tears.

According to another embodiment of the invention, the method, systems and diagnosis can be carried out post mortem on a biological sample from a deceased subject. In some embodiments, such biological samples can be pre-treated to extract proteins therefrom, including those that would be present in the blood of the deceased, so as to ensure that the relevant biomarker proteins specified above will be present in a positive sample. For the purposes of this patent specification, such an extract is equivalent to a body fluid.

Biological fluid samples, particularly peripheral biological fluid samples may be tested without prior processing of the sample as allowed by some assay formats. Alternatively, many peripheral biological fluid samples will be processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood. In some examples, the peripheral biological fluid sample is collected in a container comprising EDTA.

Subjects may be a mammal, such as a human, or a non-human subject.

In some embodiments, the human biological sample can be stored, for example as frozen biological sample prior to subjecting to the detection of levels of biomarkers as disclosed herein using the methods, kits, machines, computer systems and media as disclosed herein.

### Detection of Protein Levels of Biomarkers Beta-2-Microglobulin, CRP and Cystatin C

One can use any proteomic approach commonly known to persons of ordinary skill in the art to measure the level of the panel of biomarker proteins, (e.g., beta-2-microglobulin, CRP and cystatin C) in a biological sample.

Detection of biomarkers B2M, CRP and cystatin C can be accomplished by any suitable method. Exemplary detection methods include immunodetection methods, optical methods, electrochemical methods (voltametry and amperometry techniques), atomic force microscopy, and radio frequency methods, e.g., multipolar resonance spectroscopy. In general, it will be understood that it is normally desirable that when assessing a subject's PAD status, B2M and CRP are detected using the same category of detection method.

Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Biochips find use in exemplary methods for detection of biomarkers B2M, CRP and cystatin C in a sample. A biochip generally comprises a solid substrate having a substantially planar surface, to which a capture reagent (e.g., an adsorbent or affinity reagent) is attached. Frequently, the surface of a biochip comprises a plurality of addressable locations having bound capture reagent bound. The biochip may also include bound capture reagent that serves as a control (e.g., having a bound to biomarkers B2M, CRP and cystatin C).

Protein biochips are biochips adapted for the capture of polypeptides. Many protein biochips are described in the art. These include, for example, protein biochips produced by CIPHERGEN BIOSYSTEMS™, Inc. (Fremont, Calif.), ZYOMYX™ (Hayward, Calif.), INVITROGEN™ (Carlsbad, Calif.), BIACORE™ (Uppsala, Sweden) and PROCOGNIA™ (Berkshire, UK). Examples of such protein biochips are described in the following patents or published patent applications: U.S. Pat. No. 6,225,047 (Hutchens &Yip); U.S. Pat. No. 6,537,749 (Kuimelis and Wagner); U.S. Pat. No. 6,329,209 (Wagner et al.); PCT International Publication No. WO 00156934 (Englert et al.); PCT International Publication No. WO 031048768 (Boutell et al.) and U.S. Pat. No. 5,242,828 (Bergstrom et al.).

Detection of biomarkers B2M, CRP and cystatin C can be conducted in the same or different blood samples, the same or separate assays, and may be conducted in the same or different reaction mixture. Where biomarkers B2M, CRP and cystatin C are assayed in different blood samples, the samples are usually obtained from the subject during the same blood draw or with only a relative short time intervening so as to avoid an incorrect result due to passage of time. Where biomarkers B2M, CRP and cystatin C are detected in separate assays, the samples assayed are can be derived from the same or different blood samples obtained from the subject to be tested. Where biomarkers B2M, CRP and cystatin C are assayed in the same reaction mixture in an immunoassay, detection of biomarkers B2M, CRP and cystatin C in the sample can be accomplished using, for example, antibodies having different, detectably distinct labels so that one can distinguish between detection of specific immunocomplexes containing B2M and specific immunocomplexes containing CRP and specific immunocomplexes containing cystatin C. For example, the primary anti-B2M and anti-CRP, and anti-cystatin C antibodies can have different detectable labels (e.g., different optically detectable labels that provide for different excitation and/or emission wavelengths). In another example, the secondary antibody specific for the primary anti-B2M and the secondary antibody specific for the anti-CRP antibody and the secondary antibody for anti-cystatin C are differently detectably labeled.

Other variations of the assays described herein to provide for different assay formats for detection of biomarkers B2M, CRP and cystatin C will be readily apparent to the ordinarily skilled artisan upon reading the present disclosure. Method for immunodetection of biomarkers B2M, CRP and cystatin C are disclosed in U.S. Pat. Nos. 8,227,201 and 7,998,743 which are incorporated herein in their entirety by reference.

As described herein, the level of the panel of biomarker proteins, (e.g., beta-2-microglobulin, CRP and cystatin C) can be measured in a biological sample from a subject. The level of the panel of biomarker proteins (e.g., beta-2-microglobulin, CRP and cystatin C) can be measured using any available measurement technology that is capable of specifically determining the level of the biomarker proteins, (e.g., beta-2-microglobulin, CRP and cystatin C) in a biological sample. The measurement may be either quantitative or qualitative, so long as the measurement is capable of indicating whether the level of the panel of biomarker proteins, (e.g., beta-2-microglobulin, CRP and cystatin C) in the biological fluid sample is the same as, or above or below the reference threshold value for each biomarker protein measured.

The measured level of the biomarker proteins (e.g., beta-2-microglobulin, CRP and cystatin C) may be a primary measurement of the level of biomarker protein measuring the quantity of the biomarker protein itself, such as by detecting the number of biomarker protein molecules in the sample) or it may be a secondary measurement of the biomarker (a measurement from which the quantity of the biomarker protein can be but not necessarily deduced, such as a measure of enzymatic activity or a measure of nucleic acid, such as mRNA, encoding the biomarker protein). Qualitative data may also be derived or obtained from primary measurements.

Commonly, biomarker protein levels may be measured using an affinity-based measurement technology. “Affinity” as relates to an antibody is a term well understood in the art and means the extent, or strength, of binding of antibody to the binding partner, such as a biomarker as described herein (or epitope thereof). Affinity may be measured and/or expressed in a number of ways known in the art, including, but not limited to, equilibrium dissociation constant (KD or Kd), apparent equilibrium dissociation constant (KD′ or Kd′), and IC50 (amount needed to effect 50% inhibition in a competition assay; used interchangeably herein with “150”). It is understood that, for purposes of this invention, an affinity is an average affinity for a given population of antibodies which bind to an epitope.

Affinity-based measurement technology utilizes a molecule that specifically binds to the biomarker protein being measured (an “affinity reagent,” such as an antibody or aptamer), although other technologies, such as spectroscopy-based technologies (e.g., matrix-assisted laser desorption ionization-time of flight, MALDI-TOF spectroscopy) or assays measuring bioactivity (e.g., assays measuring mitogenicity of growth factors) may be used. Affinity-based technologies may include antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

Immunoassay technology may include any immunoassay technology which can quantitatively or qualitatively measure the level of the biomarker protein in a biological sample. Suitable immunoassay technology includes, but is not limited to radioimmunoassay, immunofluorescent assay, enzyme immunoassay, chemiluminescent assay, ELISA, immuno-PCR, and western blot assay. Likewise, aptamer-based assays which can quantitatively or qualitatively measure the level of a biomarker in a biological sample may be used in the methods of the invention. Generally, aptamers may be substituted for antibodies in nearly all formats of immunoassay, although aptamers allow additional assay formats (such as amplification of bound aptamers using nucleic acid amplification technology such as PCR (U.S. Pat. No. 4,683,202) or isothermal amplification with composite primers (U.S. Pat. Nos. 6,251,639 and 6,692,918).

Any immunoassay techniques commonly known in the art can be used in the systems and methods as disclosed herein, and include, for example, radioimmunoassay, ELISA (enzyme-linked immunosorbant assay), “sandwich” immunoassays, immunoradiometric assays, immunodiffusion assays, in situ immunoassays (using colloidal gold, enzyme or radioisotope labels, for example), western blot analysis, immunoprecipitations, immunofluorescence assays, immunoelectrophoresis assays, fluoroimmunoassay (FiA), immunoradiometric assay (IRMA), immunoenzymometric assay (IEMA), immunoluminescence assay and immunofluorescence assay (Madersbacher S, Berger P. Antibodies and immunoassays. Methods 2000; 21:41-50).

A wide variety of affinity-based assays are also known in the art. Affinity-based assays will utilize at least one epitope derived from the biomarker protein, and many affinity-based assay formats utilize more than one epitope (e.g., two or more epitopes are involved in “sandwich” format assays; at least one epitope is used to capture the biomarker protein, and at least one different epitope is used to detect the marker).

Affinity-based assays may be in competition or direct reaction formats, utilize sandwich-type formats, and may further be heterogeneous (e.g., utilize solid supports) or homogenous (e.g., take place in a single phase) and/or utilize immunoprecipitation. Many assays involve the use of labeled affinity reagent (e.g., antibody, polypeptide, or aptamer); the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA and ELONA assays. For example, the biomarker concentrations from biological fluid samples may be measured by LUMINEXO assay or ELISA, as described in Example 2 and 3. Either of the biomarker or reagent specific for the biomarker can be attached to a surface and levels can be measured directly or indirectly.

In some embodiments, one can use an immunoassay to measure the level of biomarker protein in a biological sample, for example, an ELISA method to measure biomarker protein levels using methods commonly known in the art and are encompassed for use in the present invention.

In some embodiments, a method of determining the presence and/or amount of a biomarker protein in a biological sample from a subject comprises performing a binding assay. Any reasonably specific binding partner can be used. In some embodiments, the binding partner is labeled. In some embodiments, the assay is an immunoassay, especially between the panel of biomarker proteins, (e.g., beta-2-microglobulin, CRP and cystatin C) and an antibody that recognizes each biomarker protein, especially a labeled antibody. It can be an antibody raised against part or all of it, most preferably a monoclonal antibody or a polyclonal anti-human antiserum of high specificity for human biomarker protein.

In some embodiments, an immunoassay is carried out by measuring the extent of the protein/antibody interaction of the biomarker/antibody interaction. Any known method of immunoassay may be used. A sandwich assay or ELISA is preferred. In this method, a first antibody to the marker protein is bound to the solid phase such as a well of a plastics microtitre plate, and incubated with the sample and with a labeled second antibody specific to the protein to be assayed. Alternatively, an antibody capture assay could be used. In some embodiments, a biological test sample is allowed to bind to a solid phase, and the anti-biomarker protein antibody (e.g., antibodies that specifically bind beta-2-microglobulin or CRP or cystatin C) can be added and allowed to bind. After washing away unbound material, the amount of antibody bound to the solid phase is determined using a labeled second antibody, anti- to the first.

In some embodiments, a label is preferably an enzyme. The substrate for the enzyme may be, for example, color-forming, fluorescent or chemiluminescent.

In some embodiments, a binding partner, e.g. an antibody or a ligand binding to the biomarker in the binding assay is preferably a labeled specific binding partner, but not necessarily an antibody. The binding partner will usually be labeled itself, but alternatively it may be detected by a secondary reaction in which a signal is generated, e.g. from another labeled substance.

Thus, the antibody which specifically binds to a biomarker (e.g. an antibody which binds to beta-2-microglobulin or CRP or cystatin C) can be used in the method to determine the presence and/or amount of the panel of biomarker proteins, (e.g., beta-2-microglobulin, CRP and cystatin C) in a biological sample, which can be used to detect the increased or decreased concentration of the panel of biomarker proteins, (e.g., beta-2-microglobulin, CRP and cystatin C) present in a diagnostic sample. Such antibodies can be raised by any of the methods well known in the immunodiagnostics field.

The antibodies may be anti-biomarker antibodies to any biologically relevant state of the protein. Thus, for example, they could be raised against the unglycosylated form of a biomarker protein which exists in the body in a glycosylated form, against a more mature form of a precursor protein, e.g. minus its signal sequence, or against a peptide carrying a relevant epitope of the marker protein.

In some embodiments, one can use an amplified form of assay, whereby an enhanced “signal” is produced from a relatively low level of protein to be detected. One particular form of amplified immunoassay is enhanced chemiluminescent assay. Conveniently, the antibody is labeled with horseradish peroxidase, which participates in a chemiluminescent reaction with luminol, a peroxide substrate and a compound which enhances the intensity and duration of the emitted light, typically 4-iodophenol or 4-hydroxycinnamic acid.

In another embodiment, an amplified immunoassay can be used which is immuno-PCR. In this technique, the antibody is covalently linked to a molecule of arbitrary DNA comprising PCR primers, whereby the DNA with the antibody attached to it is amplified by the polymerase chain reaction. See E. R. Hendrickson et al., Nucleic Acids Research 23: 522-529 (1995). The signal is read out as before.

Accordingly, in all aspects of the present invention, the level of a biomarker protein can be determined using a protein-binding agent, also referred to herein as “protein-binding entity” or an “affinity reagent” can be used, in particular, antibodies. For instance, the affinity reagents, in particular, antibodies such as anti-biomarker antibodies can be used in an immunoassay, particularly in an ELISA (Enzyme Linked Immunosorbent Assay). In embodiments where the level of a biomarker protein can be measured in a biological sample using methods commonly known in the art, and including, for example but not limited to isoform-specific chemical or enzymatic cleavage of isoform proteins, immunoblotting, immunohistochemical analysis, ELISA, and mass spectrometry.

As mentioned above, level of a biomarker protein can be detected by immunoassays, such as enzyme linked immunoabsorbant assay (ELISA), radioimmunoassay (RIA), Immunoradiometric assay (IRMA), Western blotting, immunocytochemistry or immunohistochemistry, each of which are described in more detail below Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

One of the most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In an “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen). The sample is then analyzed microscopically, most preferably by light microscopy of a sample stained with a stain that is detected in the visible spectrum, using any of a variety of such staining methods and reagents known to those skilled in the art.

Alternatively, “radioimmunoassays” can be employed. A radioimmunoassay is a technique for detecting and measuring the concentration of an antigen using a labeled (e.g. radioactively or fluorescently labeled) form of the antigen. Examples of radioactive labels for antigens include 3H, 14C, and 125I. The concentration of antigen enzyme in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

Other techniques can be used to detect the level the panel of biomarker proteins, (e.g., beta-2-microglobulin, CRP and cystatin C) in a biological sample can be performed according to a practitioner's preference, and based upon the present disclosure and the type of biological sample (i.e. plasma, urine, tissue sample etc.). One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Detectably labeled anti-biomarker antibodies or protein binding molecules can then be used to assess the level of the panel of biomarker proteins, (e.g., beta-2-microglobulin, CRP and cystatin C), where the intensity of the signal from the detectable label corresponds to the amount of biomarker protein. Levels of the amount of the panel of biomarker protein (e.g., beta-2-microglobulin, CRP and cystatin C) present can also be quantified, for example by densitometry.

In one embodiment, the level of the panel of biomarker proteins (e.g., beta-2-microglobulin, CRP and cystatin C) in a biological sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are incorporated herein in their entirety by reference.

In some embodiments, these methodologies can be combined with the machines, computer systems and media to produce an automated system for determining the level of the panel of biomarker proteins, (e.g., beta-2-microglobulin, CRP and cystatin C) in a biological sample and analysis to produce a printable report which identifies, for example, the level of the panel of biomarker proteins, (e.g., beta-2-microglobulin, CRP and cystatin C) protein in a biological sample.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait) which are incorporated herein by reference.

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361 which are incorporated herein by reference. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the level of the panel of biomarker proteins, (e.g., beta-2-microglobulin, CRP and cystatin C) will typically depend on the detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

In some embodiment of this aspect and all aspects disclosed herein, a biological sample can be monitored using radioactive labeling, in particular, to an inverse radioactive labeling, preferably with iodine isotopes. Preferably, an inverse radioactive labeling is performed using 125I and 131I isotopes. In another embodiment, a subject, for example a human subject can be subjected to a radioactive labeling, in particular, to an inverse radioactive labeling, preferably with iodine isotopes, such as but not limited to 125I and 131I isotopes.

In all aspects of the present invention, level of the panel of biomarker proteins, (e.g., beta-2-microglobulin, CRP and cystatin C) can be determined based on gel electrophoresis techniques, in particular SDS-PAGE (Sodium Dodecylsulfate Polyacrylamide Gel Elektrophoresis), especially two dimensional PAGE (2D-PAGE), preferably two dimensional SDS-PAGE (2D-SDS-PAGE). According to a particular example, the assay is based on 2D-PAGE, in particular, using immobilized pH gradients (IPGs) with a pH range preferably over pH 4-9.

In all aspects of the present invention, the level of the panel of biomarker proteins, (e.g., beta-2-microglobulin, CRP and cystatin C) can be determined can be using gel electrophoresis techniques, in particular, the above mentioned techniques may be combined with other protein separation methods, particularly methods known to those skilled in the art, in particular, chromatography and/or size exclusion. In all aspects of the present invention, the level of the panel of biomarker proteins, (e.g., beta-2-microglobulin, CRP and cystatin C) can be determined, if appropriate, using a combination of any of the above mentioned methods with a combination of detection methods which are well known to those skilled in the art, such as, but not limited to antibody detection and/or mass spectrometry.

In a further embodiment of all aspects of the present invention, the level of the panel of biomarker proteins, (e.g., beta-2-microglobulin, CRP and cystatin C) can be determined can be using mass spectrometry as disclose herein in the Examples, and in particular, MALDI (Matrix Assisted Laser Desorption/Ionization) and/or SELDI (Surface enhanced Laser Desorption/Ionization). In an alternative embodiment, resonance techniques, in particular, plasma surface resonance, can be used.

In some cases, it may be advantageous to achieve a separation of the biomarker proteins from a heterogeneous population of proteins in a biological sample for example using a means of one of the above outlined methods before cleaving the proteins. Such a cleavage step can be performed by applying enzymes, chemicals or other suitable reagents which are known to those skilled in the art. In an alternative embodiment, one may perform a cleavage step and subsequent separation of the cleaved the biomarker proteins, (e.g., beta-2-microglobulin, CRP and cystatin C) fragments, in particular, followed by, for example, measurements of the level of the panel of biomarker proteins, (e.g., beta-2-microglobulin, CRP and cystatin C) using any one of the methods, kits, machines, computer systems or media as disclosed herein. In some embodiments of this aspect of the invention, a cleaved biomarker protein fragments can be labeled and, optionally separated where the protein spots which correspond to cleaved biomarker protein fragments can be visualized by imaging techniques, for instance using the PROTEP TOPO® imaging technique.

In some embodiments, a protein-binding agents or antibodies or useful in the methods as disclosed herein bind or have affinity for a biomarker from the panel of biomarker proteins, (e.g., beta-2-microglobulin, CRP and cystatin C).

In some embodiments, protein-binding moieties such as antibodies can be utilized to detect the level of each biomarker from the panel of biomarker proteins (e.g., beta-2-microglobulin, CRP and cystatin C) by itself (i.e. individually), or when each biomarker exists in complex with other polypeptides, for example when it is complexed with a ligand or receptor. Additionally, in other embodiments, protein-binding moieties such as antibodies can be utilized to detect the presence of a biomarker protein (e.g., beta-2-microglobulin, CRP and cystatin C) when it is post-translationally modified, for example when a biomarker protein is ubiquitinated. In some embodiments, protein binding moieties such as antibodies can bind to a biomarker protein individually or in a complex, and in some embodiments a protein-binding moiety such as an antibody can be labeled with a detectable label.

In some embodiments, antibodies and protein-binding molecules are labeled. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

In all aspects of the present invention, the level of the panel of biomarker proteins (e.g., beta-2-microglobulin, CRP and cystatin C) can be determined by using immunological techniques using antibody, using common methods known by a person of ordinary skill in the art, e.g., antibody techniques such as immunohistochemistry, immunocytochemistry, FACS scanning, immunoblotting, radioimmunoassays, western blotting, immunoprecipitation, enzyme-linked immunosorbant assays (ELISA), and derivative techniques that make use of antibodies directed against the biomarker protein, or variants or derivatives thereof.

Any method to detect the panel of biomarker proteins, (e.g., beta-2-microglobulin, CRP and cystatin C) known by a person of ordinary skill in the art are useful in the methods, kits, machines and computer systems and media as disclosed herein to detect the level of the panel of biomarker proteins, (e.g., beta-2-microglobulin, CRP and cystatin C). For example, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody. Immunohistochemical assays are well known to those of skill in the art (e.g., see Jalkanen, et al., J. Cell. Biol. 101:976-985 (1985); Jalkanen, et al., J. Cell. Biol. 105:3087-3096 (1987).

In some embodiments, antibodies, polyclonal, monoclonal and chimeric antibodies useful in the methods as disclosed herein can be purchased from a variety of commercial suppliers, or may be manufactured using well-known methods, e.g., as described in Harlow et al., Antibodies: A Laboratory Manual, 2nd Ed; Cold. Spring Harbor Laboratory Press, Cold Spring Harbor, N.Y. (1988). In general, examples of antibodies useful in the present invention include anti-serine antibodies. Such antibodies can be purchased, for example, from Sigma-Aldrich, CalBiochem, Abcam, Santa-Cruz Biotechnology, novus Bio, U.S. biologicals, Millipore, LifeSpan, Abnova, Cell Signalling etc.

In some embodiments, direct labeling techniques can be used, where a labeled antibody is utilized. For indirect labeling techniques, the sample is further reacted with a labeled substance.

In some embodiments, immunocytochemistry may be utilized where, in general, tissue or cells are obtained from a subject are fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith Swintosky et al., 1997.

Immunological methods are particularly useful in the methods as disclosed herein, because they require only small quantities of biological material, and are easily performed and at multiple different locations. In some embodiments, such an immunological method useful in the methods as disclosed herein uses a “lab-on-a-chip” device, involving a single device to run a single or multiple biological samples and requires minimal reagents and apparatus and is easily performed, making the “lab-on-a-chip” devices which detect the panel of biomarker proteins, (e.g., beta-2-microglobulin, CRP and cystatin C) levels is ideal for rapid, on-site diagnostic tests to identify if the subject from whom the biological sample was obtained from is likely to have a major adverse event. In some embodiments, the immunological methods can be done at the cellular level and thereby necessitate a minimum of one cell. Alternatively, in some embodiments, one method to determine the amount or level of the panel of biomarker proteins, (e.g., beta-2-microglobulin, CRP and cystatin C) in a biological sample is to use a two dimensional gel electrophoresis to yield a stained gel and the increased or decreased concentration of the protein detected by an increased an increased or decreased intensity of a protein-containing spot on the stained gel, compared with a corresponding control or comparative gel.

In some embodiments, methods to determine the amount of the panel of biomarker proteins, (e.g., beta-2-microglobulin, CRP and cystatin C) in a biological sample does not necessarily require a step of comparison of the concentration of each biomarker protein with a control sample, but it can be carried out with reference either to a control or a comparative sample. Thus, measuring the amount of the panel of biomarker proteins, (e.g., beta-2-microglobulin, CRP and cystatin C) in a biological sample can be used to determine the stage of progression, if desired with reference to results obtained earlier from the same subject or by reference to standard reference threshold values that are considered typical of the stage of the disease. In this way, the invention can be used to determine whether, for example after treatment of the subject, the subject is at the same, or less (e.g., decreased) or higher (e.g., increased) risk of having a major adverse event. The result can lead to an additional prognosis of the risk of the subject having a major adverse event over time.

In a heterogeneous format, the assay utilizes two phases (typically aqueous liquid and solid). Typically a biomarker-specific affinity reagent is bound to a solid support to facilitate separation of the biomarker from the bulk of the biological sample. After reaction for a time sufficient to allow for formation of affinity reagent/biomarker complexes, the solid support or surface containing the antibody is typically washed prior to detection of bound polypeptides. The affinity reagent in the assay for measurement of biomarkers may be provided on a support (e.g., solid or semi-solid); alternatively, the polypeptides in the sample can be immobilized on a support or surface. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, glass and Protein A beads. Both standard and competitive formats for these assays are known in the art. Accordingly, provided herein are complexes comprising the biomarker bound to a reagent specific for the biomarker, wherein said reagent is attached to a surface. Also provided herein are complexes comprising at least one biomarker bound to a reagent specific for the biomarker, wherein said biomarker is attached to a surface.

Array-type heterogeneous assays are suitable for measuring the level of the panel of biomarker proteins, (e.g., beta-2-microglobulin, CRP and cystatin C) when the methods of the invention are practiced in utilizing multiple samples or where the panel of biomarker proteins, (e.g., beta-2-microglobulin, CRP and cystatin C) are measured with levels of other biomarker proteins. Array-type assays used in the practice of the methods of the invention will commonly utilize a solid substrate with two or more capture reagents specific for each biomarker in the panel of biomarker proteins, (e.g., capture reagents specific for each of beta-2-microglobulin, CRP and cystatin C) bound to the substrate a predetermined pattern (e.g., a grid). A biological fluid sample is applied to the substrate and biomarkers (e.g., beta-2-microglobulin, CRP and cystatin C proteins) in the sample are bound by the capture reagents. After removal of the sample (and appropriate washing), the bound biomarkers are detected using a mixture of appropriate detection reagents that specifically bind the various biomarkers. Binding of the detection reagent is commonly accomplished using a visual system, such as a fluorescent dye-based system. Because the capture reagents are arranged on the substrate in a predetermined pattern, array-type assays provide the advantage of detection of multiple biomarkers without the need for a multiplexed detection system.

In a homogeneous format the assay takes place in single phase (e.g., aqueous liquid phase). Typically, the biological sample is incubated with an affinity reagent specific for the biomarker protein in solution. For example, it may be under conditions that will precipitate any affinity reagent/antibody complexes which are formed. Both standard and competitive formats for these assays are known in the art.

In a standard (direct reaction) format, the level of biomarker/affinity reagent complex is directly monitored. This may be accomplished by, for example, determining the amount of a labeled detection reagent that forms is bound to a biomarker protein/affinity reagent complexes. In a competitive format, the amount of a biomarker protein in the sample is deduced by monitoring the competitive effect on the binding of a known amount of labeled biomarker (or other competing ligand) in the complex. Amounts of binding or complex formation can be determined either qualitatively or quantitatively.

The methods described in this patent may be implemented using any device capable of implementing the methods. Examples of devices that may be used include but are not limited to electronic computational devices, including computers of all types. When the methods described in the present invention are implemented in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods may also be provided over an electronic network, for example, over the internet, world-wide web, an intranet, or other network.

In one example, the methods described in the present invention may be implemented in a system comprising a processor and a computer readable medium that includes program code means for causing the system to carry out the steps of the methods described in the present invention. The processor may be any processor capable of carrying out the operations needed for implementation of the methods. The program code means may be any code that when implemented in the system can cause the system to carry out the steps of the methods described in the present invention. Examples of program code means include but are not limited to instructions to carry out the methods described in this patent written in a high level computer language such as C++, Java, or Fortran; instructions to carry out the methods described in the present invention written in a low level computer language such as assembly language; or instructions to carry out the methods described in the present invention in a computer executable form such as compiled and linked machine language.

Complexes comprising a biomarker (e.g., beta-2-microglobulin, CRP and cystatin C) and an affinity reagent can be detected by any of a number of known techniques known in the art, depending on the format of the assay and the preference of the user. For example, unlabeled affinity reagents may be detected with DNA amplification technology (e.g., for aptamers and DNA-labeled antibodies) or labeled “secondary” antibodies which bind the affinity reagent. Alternately, the affinity reagent may be labeled, and the amount of complex may be determined directly (as for dye-(fluorescent or visible), bead-, or enzyme-labeled affinity reagent) or indirectly (as for affinity reagents “tagged” with biotin, expression tags, and the like).

As will be understood by those of skill in the art, the mode of detection of the signal will depend on the detection system utilized in the assay. For example, if a radiolabeled detection reagent is utilized, the signal will be measured using a technology capable of quantitation of the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

When levels the panel of biomarker proteins (e.g., beta-2-microglobulin, CRP and cystatin C) are to be measured multiple times, or at different intervals, a biological sample may be divided into a number of aliquots, with separate aliquots used to measure the panel of biomarker protein levels at different concentrations and/or times (although division of the biological sample into multiple aliquots to allow multiple determinations of each biomarker level (e.g., beta-2-microglobulin, CRP and cystatin C) in a particular sample are also contemplated). Alternately the biological sample (or an aliquot therefrom) may be tested to determine the levels of biomarker protein in a single reaction using an assay capable of measuring the individual levels of the panel of biomarker proteins (e.g., beta-2-microglobulin, CRP and cystatin C) in a single assay, such as an array-type assay or assay utilizing multiplexed detection technology (e.g., an assay utilizing detection reagents labeled with different fluorescent dye markers).

It is common in the art to perform “replicate” measurements when measuring the panel of biomarker proteins (e.g., beta-2-microglobulin, CRP and cystatin C). Replicate measurements are ordinarily obtained by splitting a sample into multiple aliquots, and separately measuring the panel of biomarkers (e.g., beta-2-microglobulin, CRP and cystatin C) protein levels in separate reactions of the same assay system. Replicate measurements are not necessary to the methods of the invention, but many embodiments of the invention will utilize replicate testing, particularly duplicate and triplicate testing.

In one embodiment, a sample is analyzed by means of a biochip. A biochip generally comprises a solid substrate having a substantially planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by CIPHERGEN BIOSYSTEMS™, Inc. (Fremont, Calif.), ZYOMYX™ (Hayward, Calif.), INVITROGEN™ (Carlsbad, Calif.), BIACORE™ (Uppsala, Sweden) and PROCOGNIA™ (Berkshire, UK). Examples of such protein biochips are described in the following patents or published patent applications: U.S. Pat. No. 6,225,047 (Hutchens & Yip); U.S. Pat. No. 6,537,749 (Kuimelis and Wagner); U.S. Pat. No. 6,329,209 (Wagner et al.); PCT International Publication No. WO 00/56934 (Englert et al.); PCT International Publication No. WO 03/048768 (Boutell et al.) and U.S. Pat. No. 5,242,828 (Bergstrom et al.).

Detection by Mass Spectrometry. In some embodiments, a biomarker of this invention is detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In another embodiment, a mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI.

SELDI. In some embodiments, a preferred mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI also has been called is called “affinity capture mass spectrometry” or “Surface-Enhanced Affinity Capture” (“SEAC”). This version involves the use of probes that have a material on the probe surface that captures analytes through a non-covalent affinity interaction (adsorption) between the material and the analyte. The material is variously called an “adsorbent” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidazole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸M.

Protein biochips produced by CIPHERGEN BIOSYSTEMS™, Inc. comprise surfaces having chromatographic or biospecific adsorbents attached thereto at addressable locations. CIPHERGEN™ ProteinChip™ arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities Immobilized metal chelate ProteinChip arrays have nitrilotriacetic acid functionalities (IMAC 3 and IMAC 30) or 0-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Publication No. US 2003-0218130 A1 (Boschetti et al., “Biochips With Surfaces Coated With Polysaccharide-Based Hydrogels,” Apr. 14, 2003) and U.S. Patent Publication No. U.S. 2005-059086 A1 (Huang et al., “Photocrosslinked Hydrogel Blend Surface Coatings,” Mar. 17, 2005).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI chip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

SEND. Another method of laser desorption mass spectrometry is called Surface-Enhanced Neat Desorption (“SEND”). SEND involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of laser desorption mass spectrometry in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

SEPAR. Another version of LDI is called Surface-Enhanced Photolabile Attachment and Release (“SEPAR”). SEPAR involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

MALDI. MALDI is a traditional method of laser desorption/ionization used to analyze biomolecules such as proteins and nucleic acids. In one MALDI method, the sample is mixed with matrix and deposited directly on a MALDI chip. However, the complexity of biological samples such as serum or urine make this method less than optimal without prior fractionation of the sample. Accordingly, in certain embodiments with biomarkers are preferably first captured with biospecific (e.g., an antibody) or chromatographic materials coupled to a solid support such as a resin (e.g., in a spin column) Specific affinity materials that bind beta2-microglobulin is described above. After purification on the affinity material, the biomarkers are eluted and then detected by MALDI.

Other Forms of Ionization in Mass Spectrometry. In another method, the biomarkers are detected by LC-MS or LC-LC-MS. This involves resolving the proteins in a sample by one or two passes through liquid chromatography, followed by mass spectrometry analysis, typically electrospray ionization.

Data Analysis. Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In CIPHERGEN PROTEINCHIP® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip™ software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

General Protocol for SELDI Detection of Biomarkers for assessing risk of a major adverse event.

In some embodiments, the detection of the biomarkers of the invention is as follows. The biological sample to be tested, e.g., serum, preferably is subject to pre-fractionation before SELDI analysis. This simplifies the sample and improves sensitivity. A preferred method of pre-fractionation involves contacting the sample with an anion exchange chromatographic material, such as Q HyperD (BIOSEPRA™ SA). The bound materials are then subject to stepwise pH elution using buffers at pH 9, pH 7, pH 5 and pH 4. The fractions in which the biomarkers are eluted and various fractions containing the biomarker are collected.

A sample to be tested (preferably pre-fractionated) is then contacted with an affinity capture probe comprising an cation exchange adsorbent (preferably a CM10 PROTEINCHIPTm array (CIPHERGEN BIOSYSTEMS™, Inc.)) or an IMAC adsorbent (preferably an LMAC30 PROTEINCHIPTm array (CIPHERGEN BIOSYSTEMS™, Inc.)), again as indicated in Table 1, Table 2 and/or FIG. 3. The probe is washed with a buffer that will retain the biomarker while washing away unbound molecules (see Example 1, below). The biomarkers are detected by laser desorption/ionization mass spectrometry.

Alternatively, samples may be diluted, with or without denaturing, in the appropriate array binding buffer and bound and washed under conditions optimized for detecting each analyte.

Alternatively, if antibodies that recognize the biomarker are available, for example from DAKO, U.S. BIOLOGICAL™, CHEMICON™, ABCAM™ and GENWAY™. These can be attached to the surface of a probe, such as a pre-activated PS10 or PS20 ProteinChip array (CIPHERGEN BIOSYSTEMS™, Inc.). These antibodies can capture the biomarkers from a sample onto the probe surface. Then the biomarkers can be detected by, e.g., laser desorption/ionization mass spectrometry.

Any robot that performs fluidics operations can be used in these assays, for example, those available from TECAN™ or HAMILTONTm.

Detection by Immunoassay. In another embodiment of the invention, the biomarkers of the invention are measured by a method other than mass spectrometry or other than methods that rely on a measurement of the mass of the biomarker. In some embodiments, beta 2-microglobulin, CRP and/or cystatin-C can be measured by immunoassay Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art. Beta 2-microglobulin antibodies and methods for detecting beta 2-microglobulin using standard assays are described in the art, e.g., Hilgert et al. (Folia Biol (Praha) (1984) 30:369-76). Examples of the use of these antibodies to detect increased levels of beta 2-microglobulin in patients relative to normal patients are provided herein. Similar methods for the immunoassay detection of CRP and cystatin C are also known in the art.

This invention contemplates traditional immunoassays including, for example, sandwich immunoassays including ELISA or fluorescence-based immunoassays, other enzyme immunoassays and western blot. Nephelometry is an assay done in liquid phase, in which antibodies are in solution. Binding of the antigen to the antibody results in changes in absorbance, which is measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

The measured amount or concentration of a biomarker as disclosed herein can be standardized prior to the comparison. Based on the number of biomarkers examined, the desired sensitivity and specificity of the assay can be chosen. The standard can be an actual sample or previously-generated empirical data. The standard (e.g., reference threshold level) can be obtained from a known normal person. The known normal person can be a healthy person and can have a predetermined dietary intake for a predetermined time before sampling. Moreover, the sample can be obtained from a known normal person of the same sex as the subject. Alternatively, the biomarkers could be compared to those of a known major adverse event subject, in which case the similarity between the two samples, or the relative concentration of the biomarkers compared to a standard, would be examined. Various techniques and/or kits can be used by a medical professional for screening subject samples in order to determine the level and/or amount of a particular biomarker (e.g., beta-2 microglobulin, CRP and cystatin C) in a subject sample. Examples of such assays are described below and include, but are not limited to, an immunoassay, mass spectroscopy, chromatography, a chemical analysis, a colorimetric assay, a spectrophotometric analysis, an electrochemical analysis, and nuclear magnetic resonance. Additionally, such assays can be performed on any biological sample including whole blood, blood plasma, blood serum, cerebrospinal fluid, saliva, urine, seminal fluid, breast nipple aspirate, pancreatic fluid, and combinations thereof. These assays are chosen based on which are best suited to detect a particular analyte as well as which are best suited for use with a particular biological sample. Accordingly, multiple assays can be used to detect the desired analytes, and samples can be analyzed from one or more sources.

A biomarker (e.g., beta-2 microglobulin, CRP and cystatin C) can be detected and/or quantified by using one or more separation methods. For example, suitable separation methods may include a mass spectrometry method, such as electrospray ionization mass spectrometry (ESI-MS), ESI-MS/MS, ESI-MS/(MS)ⁿ (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)“, atmospheric pressure photoionization mass spectrometry (APPI-MS), APPI-MS/MS, and APPI-(MS)”. Other mass spectrometry methods may include, inter alia, quadrupole, fourier transform mass spectrometry (FTMS) and ion trap. Spectrometric techniques that can also be used include resonance spectroscopy and optical spectroscopy.

Other suitable separation methods include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE), or other chromatographic techniques, such as thin-layer, gas or liquid chromatography, or any combination thereof. In one embodiment, the biological sample to be assayed may be fractionated prior to application of the separation method.

Tandem linking of chromatography (for example liquid chromatography (“LC”)) and mass spectrometry (“MS”) can be useful for detecting and quantifying one or more of the analytes. LC can be used to separate the molecules, which may include an analyte, in a sample from an individual. A small amount of the sample, dissolved in a solvent, can be injected into the injection port of the LC device, which can be kept at a high temperature. The LC column of the device contains a solid substrate that can be either polar or non-polar. Because of differing polarities of the molecules in the sample, the molecules will have differing affinities for the solid substrate in the column and will elute at different times. The stronger the affinity of the molecule to the substrate, the longer the retention time of the molecule in the column. As the molecules exit the column, they enter the mass spectrometer. The mass spectrometer ionizes the molecules. In the tandem mass spectrometry mode, if the system can be standardized properly, each compound sent into a mass spectrometer fragments into ions of various masses and abundances forming a signature pattern unique to that substance. By comparing the tandem mass spectrograph of each peak to a computerized database, the computer is usually able to identify the molecules with a high degree of certainty. Alternately, or additionally, this comparison may be carried out by human inspection. Once an identity is established, the computer integrates the area under each peak and thereby determines the relative quantity of each molecule in the mixture. To the extent any of the molecules are identified as biomarker (e.g., beta-2 microglobulin, CRP and cystatin C), the amount of the biomarker can be compared with the amount of the same biomarker from a standard to determine if there is a difference.

Biomarker (e.g., beta-2 microglobulin, CRP and cystatin C) can also be detected and/or quantified by methods that do not require physical separation of the analytes themselves. For example, nuclear magnetic resonance (NMR) spectroscopy can be used to resolve a profile of an analyte from a complex mixture of molecules. An analogous use of NMR to classify tumors is disclosed in Hagberg, NMR Biomed. 11: 148-56 (1998), for example. Additional procedures include nucleic acid amplification technologies, which can be used to determine an analyte profile without physical separation of individual molecules. (See Stordeur et al., J. Immunol Methods 259: 55-64 (2002) and Tan et al., Proc. Nat'l Acad. Sci. USA 99: 11387-11392 (2002), for example.)

Levels of biomarkers (e.g., levels of beta-2 microglobulin, CRP and cystatin C) in a sample also can be detected and/or quantified, for example, by combining the analyte with a binding moiety capable of specifically binding the biomarker protein. A protein-binding moiety or protein binding molecule can include, for example, a member of a ligand-receptor pair, i.e., a pair of molecules capable of having a specific binding interaction. The binding moiety can also include, for example, a member of a specific binding pair, such as antibody-antigen, enzyme-substrate, nucleic acid-nucleic acid, protein-nucleic acid, protein-protein, or other specific binding pairs known in the art. Binding proteins may be designed which have enhanced affinity for a target. Optionally, the binding moiety may be linked with a detectable label, such as an enzymatic, fluorescent, radioactive, phosphorescent or colored particle label. The labeled complex may be detected, e.g., visually or with the aid of a spectrophotometer or other detector, and/or may be quantified.

Levels of biomarkers (e.g., levels of beta-2 microglobulin, CRP and cystatin C) can also be detected and/or quantified using gel electrophoresis techniques available in the art. In two-dimensional gel electrophoresis, molecules are separated first in a pH gradient gel according to their isoelectric point. The resulting gel then can be placed on a second polyacrylamide gel, and the molecules separated according to molecular weight (See, for example, O′Farrell J. Biol. Chem. 250: 4007-4021 (1975)). Levels of biomarkers (e.g., levels of beta-2 microglobulin, CRP and cystatin C) for major event may be detected by first isolating molecules from a sample obtained from an individual suspected of being at risk for a major adverse cardiovascular or cerebrovascular event and then separating the molecules by two-dimensional gel electrophoresis to produce a characteristic two-dimensional gel electrophoresis pattern. The pattern may then be compared with a standard gel pattern produced by separating, under the same or similar conditions, molecules isolated from the standard (e.g., healthy or major acute cardiac event subjects). The standard gel pattern may be stored in, and retrieved from, an electronic database of electrophoresis patterns. Thus, it can be determined if the amount of the biomarkers (e.g., levels of beta-2 microglobulin, CRP and cystatin C) in the subject is different from the amount in the standard. The presence of a plurality, e.g., two to fifty, biomarkers on the two-dimensional gel in an amount different than a known normal standard indicates a positive screen for a major adverse event in the individual. The assay thus permits the prediction and treatment of major adverse events.

Levels of biomarkers (e.g., levels of beta-2 microglobulin, CRP and cystatin C) can also be detected and/or quantified using any of a wide range of immunoassay techniques available in the art. For example, sandwich immunoassay format may be used to detect and/or quantify levels of biomarkers (e.g., levels of beta-2 microglobulin, CRP and cystatin C) in a sample from a subject. Alternatively, conventional immuno-histochemical procedures may be used for detecting and/or quantifying the presence of an levels of biomarkers (e.g., levels of beta-2 microglobulin, CRP and cystatin C) in a sample using one or more labeled binding proteins.

In a sandwich immunoassay, two antibodies capable of binding an analytes generally are used, e.g., one immobilized onto a solid support, and one free in solution and labeled with a detectable chemical compound. Examples of chemical labels that may be used for the second antibody include radioisotopes, fluorescent compounds, and enzymes or other molecules that generate colored or electrochemically active products when exposed to a reactant or enzyme substrate. When a sample containing the analyte is placed in this system, the analyte binds to both the immobilized antibody and the labeled antibody, to form a “sandwich” immune complex on the support's surface. The complexed analyte is detected by washing away non-bound sample components and excess labeled antibody, and measuring the amount of labeled antibody complexed to the analyte on the support's surface. Alternatively, the antibody free in solution, which can be labeled with a chemical moiety, for example, a hapten, may be detected by a third antibody labeled with a detectable moiety which binds the free antibody or, for example, the hapten coupled thereto.

Both the sandwich immunoassay and tissue immunohistochemical procedures are highly specific and very sensitive, provided that labels with good limits of detection are used. A detailed review of immunological assay design, theory and protocols can be found in numerous texts in the art, including Butt, W. R., Practical Immunology, ed. Marcel Dekker, New York (1984) and Harlow et al. Antibodies, A Laboratory Approach, ed. Cold Spring Harbor Laboratory (1988).

In general, immunoassay design considerations include preparation of antibodies (e.g., monoclonal or polyclonal antibodies) having sufficiently high binding specificity for the target to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target. The higher the antibody binding specificity, the lower the target concentration that can be detected. As used herein, the terms “specific binding” or “binding specifically” are understood to mean that the binding moiety, for example, a binding protein, has a binding affinity for the target of greater than about 10⁵ M⁻¹, more preferably greater than about 10⁷ M⁻¹.

Antibodies to an isolated target biomarker (e.g., beta-2 microglobulin, CRP and cystatin C) which are useful in assays for predicting a major adverse event in an individual may be generated using standard immunological procedures well known and described in the art. See, for example Practical Immunology, supra. Briefly, an isolated biomarker can be used to raise antibodies in a xenogeneic host, such as a mouse, goat or other suitable mammal. The biomarkers (e.g., beta-2 microglobulin, CRP and cystatin C) can be used alone or in combination, and can also be combined with a suitable adjuvant capable of enhancing antibody production in the host, and can be injected into the host, for example, by intraperitoneal administration. Any adjuvant suitable for stimulating the host's immune response may be used. A commonly used adjuvant is Freund's complete adjuvant (an emulsion comprising killed and dried microbial cells and available from, for example, Calbiochem Corp., San Diego, or Gibco, Grand Island, N.Y.). Where multiple antigen injections are desired, the subsequent injections may comprise the antigen in combination with an incomplete adjuvant (e.g., cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target and have the desired binding affinity.

Antibody binding domains also may be produced biosynthetically and the amino acid sequence of the binding domain manipulated to enhance binding affinity with a preferred epitope on the target. Specific antibody methodologies are well understood and described in the literature. A more detailed description of their preparation can be found, for example, in Practical Immunology, (supra). In addition, genetically engineered biosynthetic antibody binding sites, also known in the art as BABS or sFv's, may be used to determine if a sample contains an analyte. Methods for making and using BABS comprising (i) non-covalently associated or disulfide bonded synthetic V_{H} and V_{L} dimers, (ii) covalently linked V_{H} and V_{L} single chain binding sites, (iii) individual V_{H} or V_{L} domains, or (iv) single chain antibody binding sites are disclosed, for example, in U.S. Pat. Nos. 5,091,513; 5,132,405; 4,704,692; and 4,946,778. Furthermore, BABS having requisite specificity for the analyte can be derived by phage antibody cloning from combinatorial gene libraries (see, for example, Clackson et al. Nature 352: 624-628 (1991)). Briefly, phages, each expressing on their coat surfaces BABS having immunoglobulin variable regions encoded by variable region gene sequences derived from mice pre-immunized with an isolated analyte, or a fragment thereof, are screened for binding activity against the immobilized analyte. Phages which bind to the immobilized analyte are harvested and the gene encoding the BABS can be sequenced. The resulting nucleic acid sequences encoding the BABS of interest then may be expressed in conventional expression systems to produce the BABS protein.

### Determination of Subjects Risk of Having a Major Adverse Event

The biomarkers of the invention can be used in diagnostic tests to assess if a subject is at risk of a major adverse event, e.g., a heart attack, stroke or death.

The phrase “major adverse event” also referred to herein as “MAE” also includes a “major adverse cardiovascular event” or “MACE” and includes any distinguishable manifestation of a serious medical event occurring to the subject, when the outcome is death, life threatening, or requires initial or prolonged hospitalization. The term “life-threatening” in the definition of “serious” refers to an event in which the patient was at risk of death at the time of the event; it does not refer to an event, which hypothetically might have caused death if it were more severe. For example, a major adverse event can result in death, is life-threatening, requires inpatient hospitalization or a prolongation of existing hospitalization, results in persistent or significant disability/incapacity, is a congenital anomaly/birth defect, or requires intervention to prevent permanent impairment or damage. In particular, major adverse events require medium to long term care, are moderate to severe and unacceptable, they normally require further treatment and are serious and distressing. Major adverse events (MAE) are described in U.S. Pat. No. 8,090,562 which is incorporated herein in its entirety by reference.

The correlation of test results with a major adverse event involves applying a classification algorithm of some kind to the results to generate the status. The classification algorithm may be as simple as determining whether or not the amount of beta-2-microglobulin, CRP and cystatin-C measured is above or below a particular cut-off number (e.g., reference number). When multiple biomarkers or cardiovascular risk factors (e.g., the age, gender, blood pressure, blood sugar, and blood cholesterol) are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

### Reference Values and Control Subjects

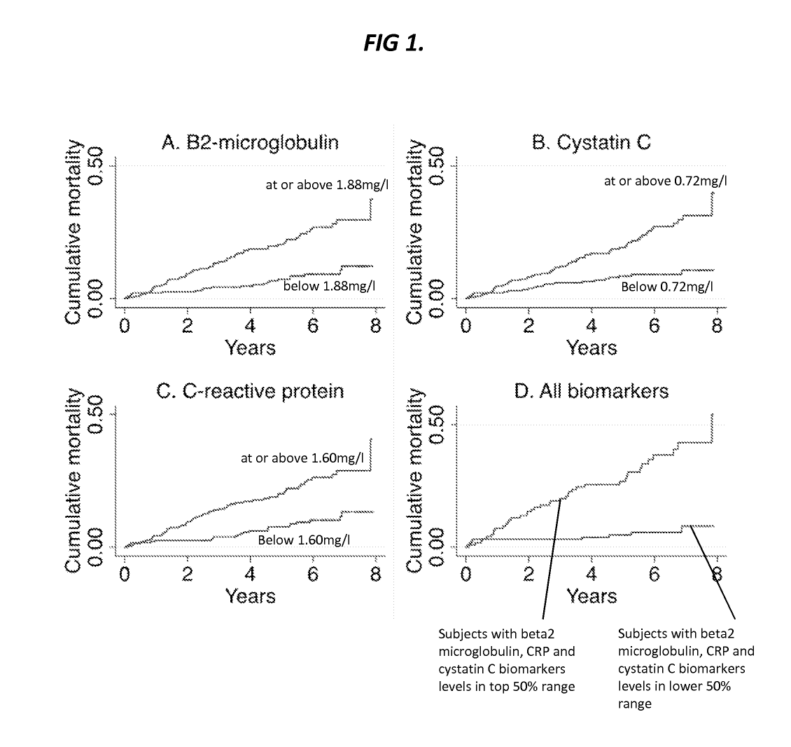

The reference threshold levels or values of biomarker levels (e.g., beta-2-microglobulin, CRP and cystatin C) used for comparison with the level of biomarker proteins, (e.g., beta-2-microglobulin, CRP and cystatin C) from a subject may vary, depending on the aspect of the invention being practiced, as will be understood throughout this specification, and below. A reference threshold value can be based on an individual sample value, such as for example, a value obtained from a biological sample from the subject being tested, but at an earlier point in time (e.g., at a first timepoint (t1), e.g., a first biomarker level measured, or at a second timepoint (t2), e.g.,). A reference threshold value can also be based on a pool of samples, for example, value(s) obtained from samples from a pool of subjects being tested. For example, as shown in FIG. 1, reference threshold values for biomarkers beta-2-microglobulin, CRP and cystatin C are based on measured the 50% value (e.g., median) of the biomarker measured in the subjects. Subjects in the top 50% (e.g., at or above the median level) for each biomarker were demonstrated to be at risk of having a major adverse event. Reference value(s) can also be based on a pool of samples including or excluding the sample(s) to be tested. The reference value can be based on a large number of samples, such as from population of healthy subjects of the chronological age-matched group, or from subjects who do not have a risk of a major adverse event.

For assessing the risk of a subject likely to experience a major adverse event by the methods and systems as disclosed herein, a “reference threshold value” is typically a predetermined reference threshold level, such as the median serum or blood biomarker protein level obtained from a population of healthy subjects that are in the chronological age group matched with the chronological age of the tested subject. As indicated earlier, in some situations, the reference samples may also be gender matched as well as matched based on ethnicity. In some embodiments, the reference threshold value for each biomarker is the median blood level for that biomarker in subjects for the same ethnicity, e.g., Caucasian, Black, Hispanic, Asian, and Asian-Indian, Pakistani, Middle Eastern and Pacific Islander.

For assessing the risk of a subject likely to experience a major adverse event using the methods and systems as disclosed herein, the reference threshold level for each biomarker may be a predetermined level, such as an average or median of levels obtained from a population of healthy subjects that are in the chronological age group matched with the chronological age of the tested subject. In some embodiments, such a predetermined level of a reference threshold level for beta 2 microglobulin (M2M) is 1.88 mg/l; the reference threshold level for cystatin C is 0.72 mg/l and the reference level for CRP is 1.60 mg/l. Alternately, the reference threshold level for each biomarker may be a historical reference level for the particular subject (e.g., a blood level of beta-2-microglobulin, and/or CRP and/or cystatin C) that was obtained from a sample derived from the same subject, but at an earlier point in time, and/or when the subject did not have a risk of a major adverse event). In some instances, the reference threshold level for each biomarker may be a historical reference level of for each biomarker for a particular group of subjects (e.g., blood levels of beta-2-microglobulin, and/or CRP and/or cystatin C from subject whom have all had a major adverse event due to coronary artery disease (CAD) etc.).

In some embodiments, control subjects are non-CAD subjects, or CAD patients who do not have a risk of a major adverse event.

In some embodiments, healthy subjects are selected as the control subjects. In some embodiments, controls are age-matched controls. Healthy subject may be used to obtain a reference threshold level of beta-2-microglobulin, and/or CRP and/or cystatin C, e.g., levels of beta-2-microglobulin, and/or CRP and/or cystatin C in a serum sample. A “healthy” subject or sample from a “healthy” subject or individual as used herein is the same as those commonly understood to one skilled in the art. For example, one may use methods commonly known to evaluate cardiac function, and/or amyloidosis to select control subjects as healthy subjects for diagnosis and treatment methods related to amyloidic cardiomyopathy. In some embodiments, subjects in good health with no signs or symptoms suggesting cardiac dysfunction can be recruited as healthy control subjects. The subjects are evaluated based on extensive evaluations consisted of medical history, family history, physical and cardiac examinations by clinicians who cardiology and/or amyloid diseases, laboratory tests. Examples of analysis of cardiac function and cardiac amyloid disease include, but are not limited to (i) electrocardiogram (ECG or EKG) which is a graphic recordation of cardiac activity, either on paper or a computer monitor. An ECG can be beneficial in detecting disease and/or damage; (ii) echocardiogram (heart ultrasound) used to investigate congenital heart disease and assessing abnormalities of the heart wall, including functional abnormalities of the heart wall, valves and blood vessels; (iiii) Doppler ultrasound (or Doppler imaging (TDI) and strain imaging (SI)) can be used to measure blood flow across a heart valve; (iv) nuclear medicine imaging (also referred to as radionuclide scanning in the art) allows visualization of the anatomy and function of an organ, and (v) magnetic resonance imaging (MRI) can be used to detect presence of amyloid deposits on organs, including the heart. In some embodiments, a control subject can be selected by lack of congo red staining or lack of anti-mycin staining of endomyocardial biopsy samples. Other methods to identify lack of cardiac amyloid deposits are known, for example, traditional echocardiographic techniques as well as new echocardiographic imaging modalities such as tissue Doppler, Doppler-based strain, speckle tracking imaging, and three-dimensional imaging in the assessment of cardiac amyloid (as disclosed in Tsang et al., Echocardiographic Evaluation of Cardiac Amyloid, Curr Cardiology Reports, 2010, 12(3), 272-276).

Age-matched populations (from which reference values may be obtained) are ideally the same chronological age as the subject or individual being tested, but approximately age-matched populations are also acceptable. Approximately age-matched populations may be within 1, 2, 3, 4, or 5 years of the chronological age of the individual tested, or may be groups of different chronological ages which encompass the chronological age of the individual being tested.

A subject that is compared to its “chronological age matched group” is generally referring to comparing the subject with a chronological age-matched within a range of 5 to 20 years. Approximately age-matched populations may be in 2, 3, 4, 5, 6, 7, 8, 9, 10 or 15, or 20 year increments (e.g. a “5 year increment” group may serve as the source for reference values for a 62 year old subject might include 58-62 year old individuals, 59-63 year old individuals, 60-64 year old individuals, 61-65 year old individuals, or 62-66 year old individuals). In a broader definition, where there are larger gaps between different chronological age groups, for example, when there are few different chronological age groups available for reference values, and the gaps between different chronological age groups exceed the 2, 3, 4, 5, 6, 7, 8, 9, 10 or 15, or 20 year increments described herein, then the “chronological age matched group” may refer to the age group that is in closer match to the chronological age of the subject (e.g. when references values available for an older age group (e.g., 80-90 years) and a younger age group (e.g., 20-30 years), a chronological age matched group for a 51 year old may use the younger age group (20-30 years), which is closer to the chronological age of the test subject, as the reference level.

Other factors to be considered while selecting control subjects include, but not limited to, species, gender, ethnicity, and so on. Moreover, biomarker reference threshold levels for beta-2-microglobulin, and/or CRP and/or cystatin C may be different within different age groups, and/or may be gender specific as well as ethnicity specific. Hence in one embodiment, a reference level may be a predetermined reference level, such as an average or median of levels obtained from a population of healthy control subjects that are gender-matched with the gender of the tested subject. In some embodiments, a reference level may be a predetermined reference level, such as an average or median of levels obtained from a population of healthy control subjects that are ethnicity-matched with the ethnicity of the tested subject (e.g., the reference threshold level for each biomarker is specific for the same ethnicity as the subject, e.g., Caucasian, Black, Hispanic, Asian, and Asian-Indian, Pakistani, Middle Eastern and Pacific Islander). In another embodiment, both chronological age and gender of the population of healthy subjects are matched with the chronological age and gender of the tested subject, respectively. In another embodiment, both chronological age and ethnicity of the population of healthy subjects are matched with the chronological age and ethnicity of the tested subject, respectively. In a further embodiment, chronological age, gender, and ethnicity of the population of healthy control subjects are all matched with the chronological age, gender, and ethnicity of the tested subject, respectively.

### Comparing Levels of the Panel of Biomarkers

The process of comparing a level of the panel of biomarkers (e.g., beta-2-microglobulin, CRP, Cystatin C) in a biological sample from a subject and a reference threshold level for each biomarker can be carried out in any convenient manner appropriate. Generally, values of biomarker levels (e.g., beta-2-microglobulin, CRP, Cystatin C) used in the methods of the invention may be quantitative values (e.g., quantitative values of concentration, such as milligrams of each biomarker per liter (e.g., mg/L) of sample, or an absolute amount). Alternatively, values of biomarker protein levels (e.g., beta-2-microglobulin, CRP, Cystatin C) level can be qualitative depending on the measurement techniques, and thus the mode of comparing a value from a subject and a reference value can vary depending on the measurement technology employed. For example, the comparison can be made by inspecting the numerical data, by inspecting representations of the data (e.g., inspecting graphical representations such as bar or line graphs). In one example, when a qualitative calorimetric assay is used to measure biomarker levels (e.g., beta-2-microglobulin, CRP, cystatin C levels), the levels may be compared by visually comparing the intensity of the colored reaction product, or by comparing data from densitometric or spectrometric measurements of the colored reaction product (e.g., comparing numerical data or graphical data, such as bar charts, derived from the measuring device).

As described herein, biological fluid samples may be measured quantitatively (absolute values) or qualitatively (relative values). In some embodiments, quantitative values of biomarker levels (e.g., beta-2-microglobulin, CRP, cystatin C levels), in the biological fluid samples may indicate a given level (or grade) of risk of a major adverse event. For example, quantitative values of biomarkers in the biological fluid samples may indicate a given level of a major adverse event.

In certain embodiments, the comparison is performed to determine the magnitude of the difference between the values from a subject and reference values (e.g., comparing the “fold” or percentage difference between the measured biomarker levels (e.g., beta-2-microglobulin, CRP, cystatin C levels), obtained from a subject and the reference threshold biomarker value). A fold difference can be determined by measuring the absolute concentration of the biomarker levels (e.g., beta-2-microglobulin, CRP, cystatin C levels), and comparing that to the absolute value to the reference threshold biomarker level, or a fold difference can be measured by the relative difference between a reference value and a sample value, where neither value is a measure of absolute concentration, and/or where both values are measured simultaneously. For example, an ELISA measures the absolute content or concentration of a protein from which a fold change is determined in comparison to the absolute concentration of the same protein in the reference. As another example, an antibody array measures the relative concentration from which a fold change is determined. Accordingly, the magnitude of the difference between the measured value and the reference value that suggests or indicates a particular diagnosis will depend on the particular biomarker being measured to produce the measured value and the reference value used (which in turn depends on the method being practiced).

As will be apparent to those of skill in the art, when replicate measurements are taken for measurement of biomarker levels (e.g., beta-2-microglobulin, CRP, cystatin C levels), the measured values from subjects can be compared with the reference threshold biomarker levels, and takes into account the replicate measurements. The replicate measurements may be taken into account by using either the mean or median of the measured values.

In some embodiments, the process of comparing may be manual (such as visual inspection by the practitioner of the method) or it may be automated. For example, an assay device (such as a luminometer for measuring chemiluminescent signals) may include circuitry and software enabling it to compare a value from a subject with a reference value for a biomarker. Alternately, a separate device (e.g., a digital computer) may be used to compare the measured biomarker levels (e.g., beta-2-microglobulin, CRP, cystatin C levels) from subject(s) and the reference threshold levels for each biomarker. Automated devices for comparison may include stored reference values for the biomarker levels (e.g., beta-2-microglobulin, CRP, cystatin C) being measured, or they may compare the measured biomarker levels from subject(s) with reference threshold levels for each biomarker that are derived from contemporaneously measured reference samples.

G-Allele of rs10757269 as a Marker for a Major Adverse Event, Including PAD

Another aspect of the present invention relates to the discovery that a polymorphism at the rs10757269 allele of the chromosome 9p21, in particular, G-allele of rs10757269 is a cardiovascular-risk and indicates a risk of PAD (peripheral Arterial Disease), a group of patients at particularly elevated risk of major adverse cardiovascular event (MACE), such as, but not limited to, myocardial infarction and stroke. In particular, the inventors have demonstrated that the panel of biomarkers (e.g., the level of beta-2-microglobulin, c-reactive protein (CRP) and cystatin C, and plasma glucose equal to, or above a reference threshold level for each biomarker and the G-allele of rs10757269), is reflective of heritable risk and proteomic information (e.g., a level of beta-2-microglobulin, c-reactive protein (CRP) and cystatin C, and plasma glucose above a predefined threshold level) integrates environmental exposures, and can be used to predict the presence or absence of PAD better than any current or established risk models.

The polymorphism rs10757269 is located on chromosome 9 near the CDKN2B gene. The polymorphism rs10757269 is present in the Homo sapiens genome and can be an A-allele or a G-Allele. The inventors have demonstrated herein that the presence of a G-allele at rs10757269 identifies a subject with an increased risk of PAD, and thus is subsequently at risk of a major adverse event.

### Single Nucleotide Polymorphisms (SNPs), Polymorphisms and Alleles

The genomes of all organisms undergo spontaneous mutation in the course of their continuing evolution, generating variant forms of progenitor genetic sequences (Gusella, Ann Rev. Biochem. 55, 831-854 (1986)). The coexistence of multiple forms of a genetic sequence gives rise to genetic polymorphisms, including SNPs.

Approximately 90% of all polymorphisms in the human genome are SNPs. SNPs are single base positions in DNA at which different alleles, or alternative nucleotides, exist in a population. The SNP position (interchangeably referred to herein as SNP, SNP site, SNP allele or SNP locus) is usually preceded by and followed by highly conserved sequences of the allele (e.g., sequences that vary in less than 1/100 or 1/1000 members of the populations). An individual can be homozygous or heterozygous for an allele at each SNP position. A SNP can, in some instances, be referred to as a “cSNP” to denote that the nucleotide sequence containing the SNP is an amino acid coding sequence.

A SNP can arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP can also be a single base insertion or deletion variant referred to as an “in/del” (Weber et al., “Human diallelic insertion/deletion polymorphisms”, Am J Hum Genet October 2002; 71(4):854-62).

A synonymous codon change, or silent mutation/SNP (the terms “SNP” and “mutation” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers”, or “di-allelic markers”.

As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore can provide increased diagnostic accuracy in some cases (Stephens et al. Science 293, 489-493, 20 Jul. 2001).

Causative SNPs are those SNPs that produce alterations in gene expression or in the expression, structure, and/or function of a gene product, and therefore are most predictive of a possible clinical phenotype. One such class includes SNPs falling within regions of genes encoding a polypeptide product, i.e. coding SNPs (or “cSNPs”). These SNPs can result in an alteration of the amino acid sequence of the polypeptide product (i.e., non-synonymous codon changes) and give rise to the expression of a defective or other variant protein. Furthermore, in the case of nonsense mutations, a SNP can lead to premature termination of a polypeptide product. Such variant products can result in a pathological condition, e.g., genetic disease. Examples of genes in which a SNP within a coding sequence causes a genetic disease include sickle cell anemia and cystic fibrosis.

Causative SNPs do not necessarily have to occur in coding regions; causative SNPs can occur in, for example, any genetic region that can ultimately affect the expression, structure, and/or activity of the protein encoded by a nucleic acid and are encompassed within the scope of the present invention. Such genetic regions include, for example, those involved in transcription, such as SNPs in transcription factor binding domains, SNPs in promoter regions, in areas involved in transcript processing, such as SNPs at intron-exon boundaries that can cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of, or predisposition to, or an increased risk in developing the disease. These SNPs, although not causative, are nonetheless also useful for diagnostics, disease predisposition screening, and other uses. In some embodiments, the G-allele of rs10757269 as disclosed herein is a causative SNP, which present in a coding region of a polypeptide or a gene.

An association study of a SNP and a specific disorder involves determining the presence or frequency of the SNP allele in biological samples from subjects with the disorder of interest, such as a subject at risk of a major adverse event (MAE) or PAD, and comparing the information to that of controls (i.e., individuals who do not have the disorder; controls can be also referred to as “healthy” or “normal” individuals) who are preferably of similar age and race. The appropriate selection of patients and controls is important to the success of SNP association studies. Therefore, a pool of individuals with well-characterized phenotypes is extremely desirable.

A SNP can be screened in diseased tissue samples or any biological sample obtained from a diseased individual, and compared to control samples, and selected for its increased (or decreased) occurrence in a specific pathological condition, such as pathologies related to coronary artery disease and coronary syndrome. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory region, etc.) that influences the pathological condition or phenotype. Association studies can be conducted within the general population and are not limited to studies performed on related individuals in affected families (linkage studies).

Particular SNP alleles, sometimes referred to as polymorphisms or polymorphic alleles, of the present invention can be associated with a risk of having PAD and thus a major adverse event.

Those skilled in the art will readily recognize that nucleic acid molecules can be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular SNP position, SNP allele, or nucleotide sequence. Probes and primers, can be designed to hybridize to either strand and SNP genotyping methods disclosed herein can generally target either strand. Throughout the specification, in identifying a SNP position, reference is generally made to the protein-encoding strand, only for the purpose of convenience.

In one aspect, the nucleic acid sequences of the gene's allelic variants, or portions thereof, can be the basis for probes or primers, e.g., in methods for determining the identity of the allelic variant of the polymorphic region. Thus, in one embodiment, nucleic acid probes or primers can be used in the methods of the present invention to determine whether a subject is at risk of a major adverse event and/or PAD a or alternatively, which therapy is most appropriate to prevent the development of the subject from having a MAE and/or PAD.

Genotyping for the G-Allele at rs10757269

According to one aspect of the present invention, a method for determining whether a human is homozygous for a polymorphism, heterozygous for a polymorphism, or lacking the polymorphism altogether (i.e. homozygous wildtype) is encompassed. As an exemplary embodiment only, method to detect the G-allele at rs10757269, a method for determining the G-allele, heterozygous for the G- and A-alleles, or homozygous for the G-allele of rs10757269 are provided. Substantially any method of detecting the G-allele at rs10757269, such as hybridization, amplification, restriction enzyme digestion, and sequencing methods, can be used.

In one embodiment, a haplotyping method useful according to the present invention is a physical separation of alleles by cloning, followed by sequencing. Other methods of haplotyping, useful according to the present invention include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotyping method which involves the use of PCR amplification.

Computational algorithms such as expectation-maximization (EM), subtraction and PHASE are useful methods for statistical estimation of haplotypes (see, e.g., Clark, A.G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. The analysis of natural populations. Genetics 120, 1145-54. (1988)).

In one embodiment, an allelic discrimination method for identifying the G-allele at rs10757269 of a human can be used. In one embodiment, the allelic discrimination method of the present invention involves use of a first oligonucleotide probe which anneals with a target portion of the individual's genome. As an illustrative example only, the target portion comprises a portion of the region surrounding rs10757269 (e.g., CTTAATTCCTTGATAGGTTCTTTTAG[A/G]TAATTTTTTTATAATGAAGCAATTA (SEQ ID NO: 1) to be screened, for example, including the nucleotide residue at position 27 in SEQ ID NO: 1. Because the nucleotide residue at this position differs, for example at position in the G-allele and the A-allele, the first probe is completely complementary to only one of the two alleles. Alternatively, a second oligonucleotide probe can also be used which is completely complementary to the target portion of the other of the two alleles. The allelic discrimination method of the present invention also involves use of at least one, and preferably a pair of amplification primers for amplifying a reference region surrounding the SNP rs10757269 of a subject. The reference region includes at least a portion of the rs10757269 SNP, for example a portion including the nucleotide residue at position 27 of the nucleic acid sequence of SEQ ID NO: 1.

The probe in some embodiments is a DNA oligonucleotide having a length in the range from about 20 to about 40 nucleotide residues, preferably from about 20 to about 30 nucleotide residues, and more preferably having a length of about 25 nucleotide residues. In one embodiment, the probe is rendered incapable of extension by a PCR-catalyzing enzyme such as Taq polymerase, for example by having a fluorescent probe attached at one or both ends thereof. Although non-labeled oligonucleotide probes can be used in the kits and methods of the invention, the probes are preferably detectably labeled. Exemplary labels include radionuclides, light-absorbing chemical moieties (e.g. dyes), fluorescent moieties, and the like. Preferably, the label is a fluorescent moiety, such as 6-carboxyfluorescein (FAM), 6-carboxy-4,7,2′,7′-tetrachlorofluoroscein (TET), rhodamine, JOE (2,7-dimethoxy-4,5-dichloro-6-carboxyfluorescein), HEX (hexachloro-6-carboxyfluorescein), or VIC.

In some embodiments, the probe of the present invention comprises both a fluorescent label and a fluorescence-quenching moiety such as 6-carboxy-N,N,N′,N′-tetramethylrhodamine (TAMRA), or 4-(4′-dimethlyaminophenylazo)benzoic acid (DABCYL). When the fluorescent label and the fluorescence-quenching moiety are attached to the same oligonucleotide and separated by no more than about 40 nucleotide residues, and preferably by no more than about 30 nucleotide residues, the fluorescent intensity of the fluorescent label is diminished. When one or both of the fluorescent label and the fluorescence-quenching moiety are separated from the oligonucleotide, the intensity of the fluorescent label is no longer diminished. In some embodiments, the probe of the present invention has a fluorescent label attached at or near (i.e. within about 10 nucleotide residues of) one end of the probe and a fluorescence-quenching moiety attached at or near the other end. Degradation of the probe by a PCR-catalyzing enzyme releases at least one of the fluorescent label and the fluorescence-quenching moiety from the probe, thereby discontinuing fluorescence quenching and increasing the detectable intensity of the fluorescent labels. Thus, cleavage of the probe (which, as discussed above, is correlated with complete complementarity of the probe with the target portion) can be detected as an increase in fluorescence of the assay mixture.

If different detectable labels are used, more than one labeled probe can be used, and therefore polymorphisms can be performed in multiplex. For example, the assay mixture can contain a first probe which is completely complementary to the target portion of the G allele of the rs10757269 loci and to which a first label is attached, and a second probe which is completely complementary to the target portion of the wildtype allele. When two probes are used, the probes are detectably different from each other, having, for example, detectably different size, absorbance, excitation, or emission spectra, radiative emission properties, or the like. For example, a first probe can be completely complementary to the target portion of the polymorphism and have FAM and TAMRA attached at or near opposite ends thereof. The first probe can be used in the method of the present invention together with a second probe which is completely complementary to the target portion of the wildtype allele and has TET and TAMRA attached at or near opposite ends thereof. Fluorescent enhancement of FAM (i.e. effected by cessation of fluorescence quenching upon degradation of the first probe by Taq polymerase) can be detected at one wavelength (e.g. 518 nanometers), and fluorescent enhancement of TET (i.e. effected by cessation of fluorescence quenching upon degradation of the second probe by Taq polymerase) can be detected at a different wavelength (e.g. 582 nanometers).

In some embodiments, the probe exhibits a melting temperature (Tm) within the range from about 60° C. to 70° C., and often within the range from 65° C. to 67° C. Furthermore, because each probe is completely complementary to only one of the alleles of rs10757269 (e.g., G- or A-allele), each probe will necessarily have at least one nucleotide residue which is not complementary to the corresponding residue of the other allele. This non-complementary nucleotide residue of the probe is often located near the midsection of the probe (i.e. within about the central third of the probe sequence) and is usually approximately equidistant from the ends of the probe. As an illustrative example, the probe which is completely complementary to the G-allele of rs10757269 can, for example, be completely complementary to nucleotide residues surrounding position 27 of SEQ ID NO: 1. For example, because the G- and A-alleles differ at position 27 of SEQ ID NO: 1, this probe will have a mismatched base pair at the nucleotide residues where the variance is, for instance a mismatch in the annealed probe at one nucleotide position corresponding with the target position of the G-allele.

By way of example, labeled probes having the sequences of SEQ ID NO:1 can be used in order to determine the allelic content of an individual (e.g. to assess whether the mammal comprises one or both of an G allele and an A allele of rs10757269). For example, custom TaqMan SNP genotyping probes for each allele can be designed using Primer Express® v2.0 software (APPLIED BIOSYSTEMS) using recommended guidelines. Successful discrimination of each allele can be verified using population control individuals. Genomic DNA (e.g. 20 ng) can be amplified according to assay recommendations and genotyping analysis performed, as described in greater detail below.

The size of the reference portion which is amplified according to the allelic discrimination method of the present invention is typically not more than about 100 nucleotide residues. It is also typical that the Tm for the amplified reference portion with the genomic DNA or fragment thereof be in the range from about 57° C. to 61° C., where possible.

It is understood that binding of the probe(s) and primers and that amplification of the reference portion of SEQ. ID NO: 1 according to the allelic discrimination method of the present invention will be affected by, among other factors, the concentration of Mg⁺⁺ in the assay mixture, the annealing and extension temperatures, and the amplification cycle times. Optimization of these factors requires merely routine experimentation which are well known to skilled artisans.

Another allelic discrimination method suitable for use in the present invention employs “molecular beacons”. Detailed description of this methodology can be found in Kostrikis et al., Science 1998; 279:1228-1229, which is incorporated herein by reference.

The use of microarrays comprising a multiplicity of reference sequences is becoming increasingly common in the art. Accordingly, another aspect of the present invention comprises a microarray having at least one oligonucleotide probe, as described above, appended thereon.

It is understood, however, that any method of ascertaining an allele of rs10757269 can be used herein. Thus, the present invention includes known methods (both those described herein and those not explicitly described herein) and allelic discrimination methods which can be hereafter developed.

As used herein, a first region of an oligonucleotide “flanks” a second region of the oligonucleotide if the two regions are adjacent one another or if the two regions are separated by no more than about 1000 nucleotide residues, and preferably no more than about 100 nucleotide residues.

A second set of primers is “nested” with respect to a first pair of primers if, after amplifying a nucleic acid using the first pair of primers, each of the second pair of primers anneals with the amplified nucleic acid, such that the amplified nucleic acid can be further amplified using the second pair of primers.

Nucleic acid molecules of the present invention can be prepared by two general methods: (1) Synthesis from appropriate nucleotide triphosphates, or (2) Isolation from biological sources. Both methods utilize protocols well known in the art.

The availability of nucleotide sequence information, such as a full-length nucleic acid sequence of SEQ ID NO: 1 or 9p21 or a part or fragment thereof, enables preparation of isolated nucleic acid molecules of the present invention by oligonucleotide synthesis. Synthetic oligonucleotides can be prepared by the phosphoramidite method employed in the APPLIED BIOSYSTEMS™ 38A DNA Synthesizer or similar devices. The resultant construct can be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Thus, for example, a 1.4 kb double-stranded molecule can be synthesized as several smaller segments of appropriate complementarity. Complementary segments thus produced can be annealed such that each segment possesses appropriate cohesive termini for attachment of an adjacent segment. Adjacent segments can be ligated by annealing cohesive termini in the presence of DNA ligase to construct an entire 1.4 kb double-stranded molecule. A synthetic DNA molecule so constructed can then be cloned and amplified in an appropriate vector.

Nucleic acid sequences of the present invention can also be isolated from appropriate biological sources using methods known in the art.

Also contemplated with the scope of the present invention are vectors or plasmids comprising a nucleic acid sequence of SEQ. ID NO:1 or fragment thereof comprising position 27 of SEQ ID NO: 1 and host cells or animals containing such vectors or plasmids. Also encompassed within the scope of the present invention are vectors or plasmids containing the nucleic acid sequences of portions of the nucleic acid sequences of SEQ ID NO:1 (e.g., containing position 27 of SEQ ID NO:1), and host cells or animals containing such vectors or plasmids. Methods for constructing vectors or plasmids containing the nucleic acid sequence of SEQ ID NO:1, or fragments thereof and host cells or animals containing the same are within the ability of persons skilled in the art of molecular biology.

Nucleic acids. Certain embodiments of the present invention concern various nucleic acids, including promoters, amplification primers, oligonucleotide probes and other nucleic acid elements involved in the analysis of genomic DNA. In certain aspects, a nucleic acid comprises a wild type, a mutant and/or a polymorphic nucleic acid.

Detection of Variances Mutations and/or Polymorphisms in rs10757269.

The rs10757269 polymorphism of the present invention can be detected directly or indirectly using any of a variety of suitable methods including fluorescent polarization, mass spectroscopy, and the like. Suitable methods comprise direct or indirect sequencing methods, restriction site analysis, hybridization methods, nucleic acid amplification methods, gel migration methods, the use of antibodies that are specific for the proteins encoded by the different alleles of the polymorphism, or by other suitable means. Alternatively, many such methods are well known in the art and are described, for example in T. Maniatis et al., Molecular Cloning, a Laboratory Manual, 2nd Edition, Cold Spring Harbor Press, Cold Spring Harbor, N.Y. (1989), J. W. Zyskind et al., Recombinant DNA Laboratory Manual, Academic Press, Inc., New York (1988), and in R. Elles, Molecular Diagnosis of Genetic Diseases, Humana Press, Totowa, N.J. (1996), and Mamotte et al, 2006, Clin Biochem Rev, 27; 63-75) each herein incorporated by reference.

According to the present invention, any approach that detects mutations or polymorphisms in a gene can be used, including but not limited to single-strand conformational polymorphism (SSCP) analysis (Orita et al. (1989) Proc. Natl. Acad. Sci. USA 86:2766-2770), heteroduplex analysis (Prior et al. (1995) Hum. Mutat. 5:263-268), oligonucleotide ligation (Nickerson et al. (1990) Proc. Natl. Acad. Sci. USA 87:8923-8927) and hybridization assays (Conner et al. (1983) Proc. Natl. Acad. Sci. USA 80:278-282). Traditional Taq polymerase PCR-based strategies, such as PCR-RFLP, allele-specific amplification (ASA) (Ruano and Kidd (1989) Nucleic Acids Res. 17:8392), single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), are easily performed and highly sensitive methods to determine haplotypes of the present invention (Michalatos-Beloin et al. (1996) Nucleic Acids Res. 24:4841-4843; Barnes (1994) Proc. Natl. Acad. Sci. USA 91:5695-5699; Ruano and Kidd (1991) Nucleic Acids Res. 19:6877-6882).

### Restriction Enzyme Analysis

In one embodiment, restriction enzymes can be utilized to identify variances in rs10757269 or a polymorphic site using “restriction fragment length polymorphism” (RFLP) analysis (Lentes et al., Nucleic Acids Res. 16:2359 (1988); and C. K. McQuitty et al., Hum. Genet. 93:225 (1994)). In RFLP, at least one target polynucleotide is digested with at least one restriction enzyme and the resulting restriction fragments are separated based on mobility in a gel. Typically, smaller fragments migrate faster than larger fragments. Consequently, a target polynucleotide that contains a particular restriction enzyme recognition site will be digested into two or more smaller fragments, which will migrate faster than a larger fragment lacking the restriction enzyme site. Knowledge of the nucleotide sequence of the target polynucleotide, the nature of the polymorphic site, and knowledge of restriction enzyme recognition sequences guide the design of such assays. In another embodiment of the present invention, restriction site analysis of particular nucleotide sequence by restriction enzymes the identity of a nucleotide at a polymorphic site is determined by the presence or absence of a restriction enzyme site. A large number of restriction enzymes are known in the art and, taken together, they are capable of recognizing at least one allele of many polymorphisms. Allele-specific Amplification (ASA).

Allele-specific Amplification is also known as amplification refectory mutation system (ARMS) uses allele specific oligonucleotides (ASO) PCR primers and is well established and known PCR based method for genotyping (Newton et al, J Med Genet, 1991; 28; 248-51). Typically, one of the two oligonucleotide primers used for the PCR binds to the mutation site, and amplification only takes place if the nucleotide of the mutation is present, with a mismatch being refractory to amplification. The resulting PCR products can be analyzed by any means known to persons skilled in the art. In a variation of the approach, termed mutagenically separated PCR (MS-PCR) the two ARMS primer of different lengths, one specific for the normal gene and one for the mutation are used, to yield PCR products of different lengths for the normal and mutant alleles (Rust et al, Nucl Acids Res, 1993; 21; 3623-9). Subsequent gel electrophoresis, for example will show at least one of the two allelic products, with normal, mutant or both (heterozygote) genes. A further variation of this forms the basis of the MASSCODE SYSTEM™ which uses small molecular weight tags covalently attached through a photo-cleavable linker to the ARMS primers, with each ARMS primers labeled with a tag of differing weight (Kokoris et al, 2000, 5; 329-40). A catalogue of numerous tags allows simultaneous amplification/genotyping (multiplexing) of 24 different targets in a single PCR reaction. For any one mutation, genotyping is based on comparison of the relative abundance of the two relevant mass tags by mass spectrometry.

### Ligation Based Assays

A number of approaches use DNA ligase, an enzyme that can join two adjacent oligonucleotides hybridized to a DNA template. In Oligonucleotide ligation assay (OLA) the sequence surrounding the mutation site is first amplified and one strand serves as a template for three ligation probes, two of these are ASO (allele-specific oligonucleotides) and a third common probe. Numerous approaches cane be used for the detection of the ligated products, for example the ASOs with differentially labeled with fluorescent of hapten labels and ligated products detected by fluorogenic of colorimetric enzyme-linked immunosorbent assays (Tobe et al, Nucleic Acid Res, 1996; 24; 3728-32). For electrophorosis-based systems, use of a morbidity modifier tags or variation in probe length coupled with fluorescence detection enables the multiplex genotyping of several single nucleotide substitutions in a single tube (Baron et al, 1997; Clinical Chem., 43; 1984-6). When used on arrays, ASOs can be spotted at specific locations or addresses on a chip, PCR amplified DNA can then be added and ligation to labeled oligonucleotides at specific addresses on the array measured (Zhong et al, Proc Natl Acad Sci 2003; 100; 11559-64).

### Single-Base Extension

Single base-extension or minisequencing involves annealing an oligonucleotide primer to the single strand of a PCR product and the addition of a single dideoxynucleotide by thermal DNA polymerase. The oligonucleotide is designed to be one base short of the mutation site. The dideoxynucleotide incorporated is complementary to the base at the mutation site. Approaches can use different fluorescent tags or haptens for each of the four different dideoxynucleotides (Pastinen et al, Clin Chem 1996, 42; 1391-7). The dideoxynucleotides differ in molecular weight and this is the basis for single-base extension methods utilizing mass-spectrometry. Genotyping based on the mass of the extended oligonucleotide primer, can be used, for example matrix-assisted laser adsorption/ionization time-of flight mass spectrometry or MALDI-TOF (Li et al, Electrophorosis, 1999, 20; 1258-65), which is quantitative and can be used to calculate the relative allele abundance making the approach suitable for other applications such as gene dosage studies (for example for estimation of allele frequencies on pooled DNA samples).

Minisequencing or Microsequencing by MALDI-TOF can be performed by means known by persons skilled in the art. In a variation of the MALDI-TOF technique, some embodiments can use the SEQUENOM™ Mass Array Technology (Sauser et al, Nucleic Acid Res, 2000, 28; E13 and Sauser et al, Nucleic Acid Res 2000, 28: E100). and also the GOOD Assay (Sauer S et al, Nucleic Acid Res, 2000; 28, E13 and Sauer et al, Nucleic Acid Res, 2000; 28:E100).

In some embodiments, variations of MALDI-TOF can be performed for analysis of variances in the rs10757269 loci. For example, MALDI and electrospray ionization (ESI) (Sauer S. Clin Chem Acta, 2006; 363; 93-105) is also useful with the methods of the present invention.

### Hybridization Based Genotyping

The G-allele at the rs10757269 loci can also be detected by measuring the binding of allele-specific oligonucleotides (ASO) hybridization probes. In such embodiments, two ASO probes, one complementary to the normal allele and the other to the mutant allele are hybridized to PCR-amplified DNA spanning the mutation site. In some embodiments, the amplified products can be immobilized on a solid surface and hybridization to radiolabelled oligonucleotides such as known as a ‘dot-blot’ assay. In alternative embodiments, the binding of the PCR products containing a quantifiable label (e.g., biotin or fluorescent labels) to a solid phase allele-specific oligonucleotide can be measured. Alternatively, for a reverse hybridization assay, or “reverse dot-blot” the binding of PCR products containing a quantifiable label (for example but not limited to biotin or fluorescent labels) to a solid phase allele-specific oligonucleotide can be measured. In some embodiments, the use of microarrays comprising hundreds of ASO immobilized onto a solid support surfaces to form an array of ASP can also be used for large scale genotyping of multiple single polymorphisms simultaneously, for example AFFYMETRIX GENECHIP® Mapping 10K Array, which can easily be performed by persons skilled in the art.

### Homogenous Assays

In homogenous assays, also called “closed tube” arrays, genomic DNA and all the reagents required for the amplification and genotyping are added simultaneously. Genotyping of the G-allele at the rs10757269 loci can be achieved without any post-amplification processing. In some embodiments, one such homogenous assay is the 5′fluorogenic nuclease assay, also known as the TaqMan® Assay (Livak et al, Genet Anal, 1999; 14:143-9) and in alternative embodiments Melting curve analyses of FRET probes are used. Such methods are carried out using “real-time” thermocyclers, and utilize two dual-labeled ASO hybridization probes complementary to normal and mutant alleles, where the two probes have different reported labels but a common quencher dye. In such embodiments, the changes in fluorescence characteristics of the probes upon binding to PCR products of target genes during amplification enables “real-time” monitoring of PCR amplification and differences in affinity of the fluorogenic probes for the PCR products of normal and mutant genes enables differentiation of genotypes. The approach uses two dual-labeled ASO hybridization probes complementary to the mutant and normal alleles. The two probes have different fluorescent reported dyes but a common quencher dye. When intact, the probes do not fluoresce due to the proximity of the reporter and quencher dyes. During annealing phase of PCR, two probes compete for hybridization to their target sequences, downstream of the primer sites and are subsequently cleaved by 5′ nuclease activity of Thermophilis aquaticus (Taq) polymerase as the primer is extended, resulting in the separation of the reporter dyes from the quencher. Genotyping is determined by measurement of the fluorescent intensity of the two reporter dyes after PCR amplification. Thus, when intact the probes do not fluoresce due to the proximity of the quencher dyes, whereas during the annealing phase of the PCR the probes compete for hybridization of the target sequences and the separation of one of the probes from the quencher, which can be detected.

Melting-curve analysis of FRET hybridization is another approach useful in the method of the invention. Briefly, the reaction includes two oligonucleotide probes which when in close proximity forms a fluorescent complex, where one probe often termed the “mutant sensor” probe is designed to specifically hybridizes across the mutation site and the other probe (often referred to as the “anchor probe”) hybridizes to an adjacent site. Fluorescent light is emitted by the “donor” excites the “acceptor” fluorophore creasing a unique fluorogenic complex, which only forms when the probes bind to adjacent sites on the amplified DNA. The “sensor” probe is complementary to either the normal or the mutant allele. Once PCR is complete, heating of the sample through the melting temperatures of the probe yields a fluorescent temperature curve which differs for the mutant and normal allele.

A variation of the FRET hybridization method is the LCGreen™ method, which obviates the requirement for fluorescent labeled probes altogether. LCGreen™ is a sensitive highly fluorogenic double-stranded DNA (dsDNA) binding dye that is used to detect the dissociation of unlabeled probes (Liew et al, Clin Chem, 2004; 50; 1156-64 and Zhou et al, Clin Chem, 2005; 51; 1761-2). The method uses unlabeled allele-specific oligonucleotides probes that are perfectly complementary either to the mutant or normal allele, and the mismatch of the ASO/template double strand DNA complex results in a lower melting temperature and an earlier reduction in fluorescent signal form the dsDNA binding dye with increasing temperature.

The OLA can also be used for FRET Probes (Chen et al, 1998; 8:549-56), for example, the PCR/ligation mixture can contain PCR primers, DNA polymerase without 5′ nuclease activity, thermal stable DNA ligase and oligonucleotides for the ligation reaction. The ligation of the allele-specific oligonucleotides have a different acceptor fluorophore and the third ligation oligonucleotide, which binds adjacently to the ASO has a donor fluorophore, and the three ligation oligonucleotides are designed to have a lower melting temperature for the PCR primers to prevent their interference in the PCR amplification. Following PCR, the temperature is lowered to allow ligation to proceed, which results in FRET between the donor and acceptor dyes, and alleles can be disconcerted by comparing the fluorescence emission of the two dyes.

Alternatives to homogenous PCR- and hybridization—based techniques for genotyping the G-allele at the rs10757269 loci are also encompassed. For example, molecular beacons (Tyagi et al, Nat Biotech, 1998; 16:49-53) and Scorpion® probes (Thelwell et al, Nucleic Acid Res, 2000; 28; 3752-610).

The OLA can also be performed by the use of FRET probes (Chen et al, Genome Res, 1998; 8:549-56). In such an embodiment, the PCR/ligation mix contains PCR primers, a thermostable DNA polymerase without 5′ exonuclease activity (to prevent the cleavage of ligation probes during the ligation phase), a thermostable DNA ligase as well as the oligonucleotides for the ligation reaction. The ligation of the ASO each have a different acceptor fluorophore and the third ligation oligonucleotide, which binds adjacently to the ASO has a donor fluorophore. The three ligation oligonucleotides are designed to have a lower melting temperature than the annealing temperature for the PCR primers prevent their interference in PCR amplification. Following PCR, the temperature is lowered to allow ligation to proceed. Ligation results in FRET between donor and acceptor dyes, and alleles can be discerned by comparing the fluorescence emission of the two dyes.

Further, variations of the homogenous PCR- and hybridization based techniques to detect the G-allele at the rs10757269 loci are also encompassed in the present invention. For example, the use of Molecular Beacons (Tyagi et al, Nat Biotech 1998; 16; 49-53) and Scorpion® Probes (Thelwell et al, Nucleic Acid Res 2000; 28; 3752-61). Molecular Beacons are comprised of oligonucleotides that have fluorescent reporter and dyes at their 5′ and 3′ ends, with the central portion of the oligonucleotide hybridizing across the target sequence, but the 5′ and 3′ flanking regions are complementary to each other. When not hybridized to their target sequence, the 5′ and 3′ flanking regions hybridize to form a stem-loop structure, and there is little fluorescence because of the proximity of the reported and the quencher dyes. However, upon hybridization to their target sequence, the dyes are separated and there is a large increase in the fluorescence. Mismatched probe-target hybrids dissociate at substantially lower temperatures than exactly matched complementary hybrids. There are a number of variations of the “molecular Beacon” approach. In some embodiments, such a variation includes use of Scorpion® Probes which are similar but incorporate a PCR primer sequence as part of the probe (Thelwell et al, Nucleic Acid Res 2000; 28; 3752-61). In another variation, ‘duplex’ format gives a better fluorescent signal (Solinas et al, Nucleic Acid Res, 2001, 29;E96).

In another embodiment, the G-allele at the rs10757269 loci can be detected by genotyping using a homogenous or real-time analysis on whole blood samples, without the need for DNA extraction or real-time PCR. Such a method is compatible with FRET and TaqMan® (Castley et al, Clin Chem, 2005; 51; 2025-30) enabling extremely rapid screening for the particular polymorphism of interest.

Fluorescent Polarization (FP). In FP, the degree to which the emitted light remains polarized in a particular plane is proportional to the speed at which the molecules rotate and tumble in solution. Under constant pressure, temperature and viscosity, FP is directly related to the molecular weight of a fluorescent species. Therefore, when a small fluorescent molecule is incorporated into a larger molecule, there is an increase in FP. FP can be used in for genotyping of polymorphisms of interest (Chen et al, Genome Res, 1999; 9:492-8 and Latif et al, Genome Res, 2001; 11; 436-40). FP can be utilized in 5′ nuclease assay (as described above), where the oligonucleotide probe is digested to a lower molecule weight species, for example is amenable to analysis by FP, but with the added benefit of not requiring a quencher. For example, PerkinElmer's AcycloPrime™-FP SNP Detection Kit can be used as a FP minisequencing method. Following PCR amplification, unincorporated primers and nucleotides are degraded enzymatically, the enzymes heat inactivated and a miniseqencing reaction using DNA polymerase and fluorescent-labeled dideoxynucleotides performed. FP is then measured, typically in a 96- to 386-well plate format on a FP-plate reader.

Pyrosequencing™. Pyrosequencing™ is a novel and rapid sequencing technique. It is a homogenous methods which is not based on chain termination, does not use dideoxynucleotides, nor does it require electrophorosis (Ahmadian et al, Anal Biochem, 2000, 280:103-10; Alderborn et al, Genome Res, 2000; 10:1249-58; and Ronaghi et al, Anal Biochem, 2000; 286:282-8). The approach is based on the generation of pyrophosphate whenever a deoxynucleotide is incorporated during polymerization of DNA, for example as nucleotides are added to the 3; end of a sequencing primer, or a primer extension: DNAn+dNTP 4 DNAn+1+ pyrophosphate. The generation of pyrophosphate us coupled to a luciferase catalyzed reaction resulting in light emission if the particular deoxynucleotide added is incorporated, yielding a qualitative and distinctive program. Sample processing includes PCR amplification with a biotinylated primer, isolation of the biotinylated single stranded amplicon on streptavidin coated beads (or other solid phase) and annealing of a sequencing primer. Samples are then analyzed by a Pyrosequencer™ which adds a number of enzymes and substrates required for indicator reaction, including sulfurylase and luciferase, as well as a pyrase for degradation of unincorporated nucleotides. The sample is then interrogated by addition of the four deoxynucleotides. Light emission is detected by a charge coupled device camera (CCD) and is proportional to the number of nucleotides incorporated. Results are automatically assigned by pattern recognition.

Other genotyping assays and techniques known to persons skilled in the art to detect a G-allele at the rs10757269 loci are encompassed for use with the present invention, for example see Kwok, Hum Mut 2002; 9; 315-323 and Kwok, Annu Rev Genomic Hum Genetics, 2001; 2; 235-58 for reviews, which are incorporated herein in their entirety by reference. Examples of other techniques to detect variances and/or polymorphisms are the Invader® Assay (Gut et al, Hum Mutat, 2001; 17:475-92, Shi et al, Clin Chem, 2001, 47, 164-92, and Olivier et al, Mutat Res, 2005; 573:103-110), the method utilizing FLAP endonucleases (U.S. Pat. No. 6,706,476) and the SNPlex genotyping systems (Tobler et al, J. Biomol Tech, 2005; 16; 398-406.

In one embodiment, a long-range PCR (LR-PCR) is used to detect the G-allele at the rs10757269 loci of the present invention. LR-PCR products are genotyped for mutations or polymorphisms using any genotyping methods known to one skilled in the art, and haplotypes inferred using mathematical approaches (e.g., Clark's algorithm (Clark (1990) Mol. Biol. Evol. 7:111-122).

For example, methods including complementary DNA (cDNA) arrays (Shalon et al., Genome Research 6(7):639-45, 1996; Bernard et al., Nucleic Acids Research 24(8):1435-42, 1996), solid-phase mini-sequencing technique (U.S. Pat. No. 6,013,431, Suomalainen et al. Mol. Biotechnol. June;15(2):123-31, 2000), ion-pair high-performance liquid chromatography (Doris et al. J. Chromatogr. A can 8; 806(1):47-60, 1998), and 5′ nuclease assay or real-time RT-PCR (Holland et al. Proc Natl Acad Sci USA 88: 7276-7280, 1991), or primer extension methods described in the U.S. Pat. No. 6,355,433, can be used.

In one embodiment, the primer extension reaction and analysis is performed using PYROSEQUENCING™ (Uppsala, Sweden) which essentially is sequencing by synthesis. A sequencing primer, designed directly next to the nucleic acid differing between the disease-causing mutation and the normal allele or the different SNP alleles is first hybridized to a single stranded, PCR amplified DNA template from the individual, and incubated with the enzymes, DNA polymerase, ATP sulfurylase, luciferase and apyrase, and the substrates, adenosine 5′ phosphosulfate (APS) and luciferin. One of four deoxynucleotide triphosphates (dNTP), for example, corresponding to the nucleotide present in the mutation or polymorphism, is then added to the reaction. DNA polymerase catalyzes the incorporation of the dNTP into the standard DNA strand. Each incorporation event is accompanied by release of pyrophosphate (PPi) in a quantity equimolar to the amount of incorporated nucleotide. Consequently, ATP sulfurylase converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a PYROGRAIVI™. Each light signal is proportional to the number of nucleotides incorporated and allows a clear determination of the presence or absence of, for example, the mutation or polymorphism. Thereafter, apyrase, a nucleotide degrading enzyme, continuously degrades unincorporated dNTPs and excess ATP. When degradation is complete, another dNTP is added which corresponds to the dNTP present in for example the selected SNP. Addition of dNTPs is performed one at a time. Deoxyadenosine alfa-thio triphosphate (dATPS) is used as a substitute for the natural deoxyadenosine triphosphate (dATP) since it is efficiently used by the DNA polymerase, but not recognized by the luciferase. For detailed information about reaction conditions for the PYROSEQUENCING, see, e.g. U.S. Pat. No. 6,210,891, which is herein incorporated by reference in its entirety.

Molecular beacons also contain fluorescent and quenching dyes, but FRET only occurs when the quenching dye is directly adjacent to the fluorescent dye. Molecular beacons are designed to adopt a hairpin structure while free in solution, bringing the fluorescent dye and quencher in close proximity Therefore, for example, two different molecular beacons are designed, one recognizing the mutation or polymorphism and the other the corresponding wildtype allele. When the molecular beacons hybridize to the nucleic acids, the fluorescent dye and quencher are separated, FRET does not occur, and the fluorescent dye emits light upon irradiation. Unlike TaqMan probes, molecular beacons are designed to remain intact during the amplification reaction, and must rebind to target in every cycle for signal measurement. TaqMan probes and molecular beacons allow multiple DNA species to be measured in the same sample (multiplex PCR), since fluorescent dyes with different emission spectra can be attached to the different probes, e.g. different dyes are used in making the probes for different disease-causing and SNP alleles. Multiplex PCR also allows internal controls to be co-amplified and permits allele discrimination in single-tube assays. (AMBION™ Inc, Austin, Tex., TechNotes 8(1)—February 2001, Real-time PCR goes prime time).

Another method to detect G-allele at the rs10757269 loci is by using fluorescence tagged dNTP/ddNTPs. In addition to use of the fluorescent label in the solid phase mini-sequencing method, a standard nucleic acid sequencing gel can be used to detect the fluorescent label incorporated into the PCR amplification product. A sequencing primer is designed to anneal next to the base differentiating the disease-causing and normal allele or the selected SNP alleles. A primer extension reaction is performed using chain terminating dideoxyribonucleoside triphosphates (ddNTPs) labeled with a fluorescent dye, one label attached to the ddNTP to be added to the standard nucleic acid and another to the ddNTP to be added to the target nucleic acid.

Alternatively, an INVADER® assay can be used (Third Wave Technologies, Inc (Madison, Wis.)). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535). For example, an INVADER® operating system (OS), provides a method for detecting and quantifying DNA and RNA. The INVADER® OS is based on a “perfect match” enzyme-substrate reaction. The INVADER® OS uses proprietary CLEAVASE® enzymes (Third Wave Technologies, Inc (Madison, Wis.)), which recognize and cut only the specific structure formed during the INVADER® process which structure differs between the different alleles selected for detection, i.e. the disease-causing allele and the normal allele as well as between the different selected SNPs. Unlike the PCR-based methods, the INVADER® OS relies on linear amplification of the signal generated by the INVADER® process, rather than on exponential amplification of the target.

In the INVADER® process, two short DNA probes hybridize to the target to form a structure recognized by the CLEAVASE® enzyme. The enzyme then cuts one of the probes to release a short DNA “flap.” Each released flap binds to a fluorescently-labeled probe and forms another cleavage structure. When the CLEAVASE® enzyme cuts the labeled probe, the probe emits a detectable fluorescence signal.

The G-allele at the rs10757269 loci can also be detected using allele-specific hybridization followed by a MALDI-TOF-MS detection of the different hybridization products. In the preferred embodiment, the detection of the enhanced or amplified nucleic acids representing the different alleles is performed using matrix-assisted laser desorption ionization/time-of-flight (MALDI-TOF) mass spectrometric (MS) analysis described in the Examples below. This method differentiates the alleles based on their different mass and can be applied to analyze the products from the various above-described primer-extension methods or the INVADER® process.

In one embodiment, a haplotyping method useful according to the present invention is a physical separation of alleles by cloning, followed by sequencing. Other methods of haplotyping, useful according to the present invention include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotyping method which involves the use of PCR amplification.

Computational algorithms such as expectation-maximization (EM), subtraction and PHASE are useful methods for statistical estimation of haplotypes (see, e.g., Clark, A. G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

### Other Assays

Other genotyping assays and methods for detecting the presence of the G-allele at the rs10757269 loci can be used within the scope of the present invention, for example, to detect mutations in genomic DNA, cDNA and/or RNA samples. Methods commonly used, or newly developed or methods yet unknown are encompassed for used in the present invention. Examples of newly discovered methods include for example, but are not limited to; SNP mapping (Davis et al, Methods Mol Biology, 2006; 351; 75-92); Nanogen Nano Chip, (keen-Kim et al, 2006; Expert Rev Mol Diagnostic, 6; 287-294); Rolling circle amplification (RCA) combined with circularable oligonucleotide probes (c-probes) for the detection of nucleic acids (Zhang et al, 2006: 363; 61-70), luminex XMAP system for detecting multiple SNPs in a single reaction vessel (Dunbar S A, Clin Chim Acta, 2006; 363; 71-82; Dunbar et al, Methods Mol Med, 2005; 114:147-1471) and enzymatic mutation detection methods (Yeung et al, Biotechniques, 2005; 38; 749-758).

Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single strand conformation polymorphism analysis (“SSCP”) and other methods well known in the art.

One method of screening for the G-allele at the rs10757269 loci is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

In such embodiments, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e. g., RNA or DNA, obtained from a tissue sample. The double-stranded duplexes are treated with an agent which cleaves single-stranded regions of the duplex such as duplexes formed based on base pair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNAse A treatment, electrophoretically separated according to size, are compared to similarly treated control duplexes. Samples containing smaller fragments (cleavage products) not seen in the control duplex are scored as positive.

Other investigators have described the use of RNaseI in mismatch assays. The use of RNaseI for mismatch detection is described in literature from PROMEGA BIOTECH™. PROMEGA™ markets a kit containing RNaseI that is reported to cleave three out of four known mismatches.

In other embodiments, alterations in electrophoretic mobility are used to identify the particular allelic variant. For example, single strand conformation polymorphism (SSCP) can be used to detect differences in electrophoretic mobility between mutant and wild type nucleic acids (Orita et al. (1989) Proc Natl. Acad. Sol USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

Gel Migration Single strand conformational polymorphism (SSCP; M. Orita et al., Genomics 5:8 74-8 79 (1989); Humphries et al., In: Molecular Diagnosis of Genetic Diseases, R. Elles, ed. pp321-340 (1996)) and temperature gradient gel electrophoresis (TGGE; R. M. Wartell et al., Nucl. Acids Res. 18:2699-2706 (1990)) are examples of suitable gel migration-based methods for determining the identity of a polymorphic site. In SSCP, a single strand of DNA will adopt a conformation that is uniquely dependent of its sequence composition. This conformation is usually different, if even a single base is changed. Thus, certain embodiments of the present invention, SSCP can be utilized to identify polymorphic sites, as wherein amplified products (or restriction fragments thereof of the target polynucleotide are denatured, then run on a non-denaturing gel. Alterations in the mobility of the resultant products are thus indicative of a base change. Suitable controls and knowledge of the “normal” migration patterns of the wild-type alleles can be used to identify polymorphic variants.

In yet another embodiment, the identity of the G-allele at the rs10757269 loci is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for, example by adding a GC clamp of approximately 40 bp of high-melting GC rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Others have described using the MutS protein or other DNA-repair enzymes for detection of single-base mismatches. Alternative methods for detection of deletion, insertion or substitution mutations that can be used in the practice of the present invention are disclosed in U.S. Pat. Nos. 5,849,483, 5,851,770, 5,866,337, 5,925,525 and 5,928,870, each of which is incorporated herein by reference in its entirety.

### Further Examples of SNP Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are widely used in human and animal genetic analyses.

In one embodiment, restriction enzymes can be utilized in identifying a polymorphic site in “restriction fragment length polymorphism” (RFLP) analysis (Lentes et al., Nucleic Acids Res. 16:2359 (1988); and C. K. McQuitty et al., Hum. Genet. 93:225 (1994)). In RFLP, at least one target polynucleotide is digested with at least one restriction enzyme and the resultant “restriction fragments” are separated based on mobility in a gel. Typically, smaller fragments migrate faster than larger fragments. Consequently, a target polynucleotide that contains a particular restriction enzyme recognition site will be digested into two or more smaller fragments, which will migrate faster than a larger fragment lacking the restriction enzyme site. Knowledge of the nucleotide sequence of the target polynucleotide, the nature of the polymorphic site, and knowledge of restriction enzyme recognition sequences guide the design of such assays. In another embodiment of the present invention, restriction site analysis of particular nucleotide sequence by restriction enzymes the identity of a nucleotide at a polymorphic site is determined by the presence or absence of a restriction enzyme site. A large number of restriction enzymes are known in the art and, taken together, they are capable of recognizing at least one allele of many polymorphisms.

However, such single nucleotide polymorphisms (SNPs) rarely result in changes in a restriction endonuclease site. Thus, SNPs are rarely detectable by restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset Alzheimer's disease etc.

In context of the present invention, screening methods and assays to detect G-allele at the rs10757269 loci are performed to screen an individual for the risk of a major adverse event (MAE) and/or PAD as disclosed herein. To do this, a sample (such as blood or other bodily fluid or tissue sample) will be taken from a subject for genotype analysis.

Several methods have been developed to screen polymorphisms and some examples are listed below. The reference of Kwok and Chen (2003) and Kwok (2001) provide overviews of some of these methods, both of these references are specifically incorporated by reference.

Examples of identifying polymorphisms and applying that information in a way that yields useful information regarding patients can be found, for example, in U.S. Pat. No. 6,472,157; U.S. Patent Application Publications 20020016293, 20030099960, 20040203034; WO 0180896, all of which are hereby incorporated by reference.

### Linkage Disequilibrium

Polymorphisms in linkage disequilibrium with the polymorphism at the G-allele at the rs10757269 loci can also be used with the methods of the present invention. “Linkage disequilibrium” (“LD” as used herein, though also referred to as “LED” in the art) refers to a situation where a particular combination; of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or o value that can be 0.25 or 0.1 and can be 0.1, 0.05. 0.001, 0.00001 or less. “Haplotype” is used herein according to its plain and ordinary meaning to one skilled in the art. It refers to a collective genotype of two or more alleles or polymorphisms along one of the homologous chromosomes.

The term “allele-specific PCR” refers to PCR techniques where the primer pairs are chosen such that amplification is dependent upon the input template nucleic acid containing the polymorphism of interest. In such embodiments, primer pairs are chosen such that at least one primer is an allele-specific oligonucleotide primer. In some embodiments of the present invention, allele-specific primers are chosen so that amplification creates a restriction site, facilitating identification of a polymorphic site. In other embodiments of the present invention, amplification of the target polynucleotide is by multiplex PCR (Wallace et al. (PCT Application W089/10414)). Through the use of multiplex PCR, a multiplicity of regions of a target polynucleotide can be amplified simultaneously. This is particularly advantageous in embodiments where more than one SNP is to be detected.

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e g, immunohistochemistry or immunoprecipitation. Antibodies to the wild-type or signal peptide mutated forms of the signal peptide proteins can be prepared according to methods known in the art.

In another embodiment, multiplex PCR procedures using allele-specific primers can be used to simultaneously amplify multiple regions of a target nucleic acid (PCT Application W089/10414), enabling amplification only if a particular allele is present in a sample. Other embodiments using alternative primer-guided nucleotide incorporation procedures for assaying polymorphic sites in DNA can be used, and have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Nad. Acad. Sci. (U.S.A) 88:1143-1147 (1991); Bajaj et al. (U.S. Pat. No. 5,846,710); Prezant, T. R. et al., Hum Mutat. 1: 159-164 (1992); Ugozzoli, L. et al., GATA 9:107-112 47 (1992); Nyr6n, P. et al., Anal. Biochem. 208:171-175 (1993)).

Other known nucleic acid amplification procedures include transcription-based amplification systems (Malek, L. T. et al., U.S. Pat. No. 5,130,238; Davey, C. et al., European Patent Application 329,822; Schuster et al.) U.S. Pat. No. 5,169,766; Miller, H. I. et al., PCT-Application W089/06700; Kwoh, D. et al., Proc. NatI. Acad Sci. (U.S.A) 86:1173 Z1989); Gingeras, T. R. et al., PCT Application W088/10315)), or isothermal amplification methods (Walker, G. T. et al., Proc. NatI. 4cad Sci. (U.S.A) 89:392-396 (1992)) can also be used.

### Solid Supports

Solid supports containing oligonucleotide probes for identifying the alleles, including the G-allele at the rs10757269 loci of the present invention can be filters, polyvinyl chloride dishes, silicon or glass based chips, etc. Such wafers and hybridization methods are widely available, for example, those disclosed by Beattie (WO 95/11755). Any solid surface to which oligonucleotides can be bound, either directly or indirectly, either covalently or noncovalently, can be used. A preferred solid support is a high density array or DNA chip. These contain a particular oligonucleotide probe in a predetermined location on the array. Each predetermined location can contain more than one molecule of the probe, but each molecule within the predetermined location has an identical sequence. Such predetermined locations are termed features. There can be, for example, about 2, 10, 100, 1000 to 10,000; 100,000, 400,000 or 1,000,000 of such features on a single solid support. The solid support, or the area within which the probes are attached can be on the order of a square centimeter.

Oligonucleotide probe arrays can be made and used according to any techniques known in the art (see for example, Lockchart et al. (1996), Nat. Biotechnol. 14: 1675-1680; McGall et al. (1996), Proc. Nat. Acad. Sci. USA 93: 13555-13460). Such probe arrays can contain at least two or more oligonucleotides that are complementary to or hybridize to two or more of the SNPs described herein.

### Databases

The present invention includes databases containing information concerning subjects with a G-allele at the rs10757269 loci and any associated symptoms with PAD and/or details if the subject has suffered a serious adverse event, as defined herein, for instance, information concerning polymorphic allele frequency and strength of the association of the G-allele at the rs10757269 loci with myocardial infarction, stroke and other major adverse events and the like. Databases can also contain information associated with subjects G-allele at the rs10757269 loci such as descriptive information about the probability of association of the polymorphism with prediction of clinical phenotype, for example the likelihood of the subject having PAD and/or a major adverse event and/or prediction of infarct size on myocardial infarction. Other information that can be included in the databases of the present invention include, but is not limited to, SNP sequence information, descriptive information concerning the clinical status of a tissue sample analyzed for SNP haplotype, or the subject from which the sample was derived. The database can be designed to include different parts, for instance a SNP frequency database and a SNP sequence database. Methods for the configuration and construction of databases are widely available, for instance, see Akerblom et al., (1999) U.S. Pat. No. 5,953,727, which is herein incorporated by reference in its entirety.

The databases of the present invention can be linked to an outside or external database. In a preferred embodiment, the external database can be the HGBASE database maintained by the Karolinska Institute, The SNP Consortium (TSC) and/or the databases maintained by the National Center for Biotechnology Information (NCBI) such as GenBank.

The databases of the present invention can also be used to present information identifying the polymorphic alleles in a subject and such a presentation can be used to predict the likelihood that the subject will develop cancer. Further, the databases of the present invention can comprise information relating to the expression level of one or more of the genes associated with the polymorphic alleles of the invention.

### Combinations of Markers

beta2-microglobulin, CRP and cystatin C are differentially present in predicting a subject at risk of a major adverse event, and, therefore, are each useful by themselves in methods of determining a major adverse event. The method involves, first, measuring beta2-microglobulin, CRP or cystatin C in a subject sample using the methods described herein, e.g., measurement by an immunoassay or capture on a SELDI biochip followed by detection by mass spectrometry and, second, comparing the measurement with a diagnostic amount or cut-off (e.g., reference value) that distinguishes a positive major adverse event status from a negative major adverse event status. The diagnostic amount represents a measured amount of a biomarker above which or below which a subject is classified as having a particular major adverse event status. For example, because beta-2-microglobulin, CRP and cystatin-C are all up-regulated in a subject at risk of a major adverse event compared to a normal subject (e.g., not at risk of a major adverse event), then a measured amount of beta-2-microglobulin, CRP and/or cystatin C above the diagnostic cutoff reference level indicates an increased risk of a major adverse event. By contrast, a level of CRP, beta-2-microglobulin, or cystatin C may be low enough to virtually exclude the subject being at risk of a major adverse event. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different major adverse events statuses, as was done here, and drawing the cut-off to suit the diagnostician's desired levels of specificity and sensitivity.

In some embodiments, the cut-off levels are shown in FIG. 1. For example, in some embodiments, the cut off reference level for beta 2 macroglobulin is 1.88 mg/l in the blood, where a subject with a level of beta-2 microglobulin at or above 1.88 mg/l is at risk of having a major adverse event. In some embodiments, the cut off reference level for cystatin C is 0.72 mg/l in the blood, where a subject with a level of cystatin-c at or above 0.72 mg/l is at risk of having a major adverse event. In some embodiments, the cut off reference level for CRP is 1.66 mg/l in the blood, where a subject with a level of CRP at or above 1.60 mg/l is at risk of having a major adverse event.

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test. A combination of at least two biomarkers is sometimes referred to as a “biomarker profile” or “biomarker fingerprint.” Accordingly, beta-2-microglobulin, CRP and cystatin-C can be combined with other biomarkers for detecting major adverse events to improve the sensitivity and/or specificity of the diagnostic test. Examples of other biomarkers useful for screening for major adverse events are disclosed in U.S. Pat. No. 8,090,562, which is incorporated herein in its entirety by reference.

### Major Adverse Event Status

Determining a major adverse event status typically involves classifying an individual into one of two or more groups (statuses) based on the results of the diagnostic test. The diagnostic tests described herein can be used to classify between a number of different states. The phrase “MAE status” includes distinguishing, inter alia, MAE v. non-MAE (e.g., normal).

In one embodiment, the invention provides methods, screens and assays for assessing the risk of a subject having a major adverse event (status: MAE v. non-MAE). The risk of a major adverse event is determined by measuring the relevant biomarkers (e.g., beta-2-microglobulin, CRP and cystatin-C alone or in combination with other biomarkers) and then either submitting them to a classification algorithm or comparing them with a reference amount (e.g., a cut off reference amount as disclosed herein) and/or pattern of biomarkers that is associated with the particular risk level.

### Determining Risk of Having a Major Adverse Event

In one embodiment, this invention provides methods for determining a subject with a high risk of having a major adverse event (status: low-risk v. high risk). Biomarker amounts or patterns are characteristic of various risk states, e.g., high, medium or low. The risk of developing a disease is determined by measuring the relevant biomarkers (e.g., beta-2-microglobulin, CRP and cystatin-C alone or in combination with other biomarkers) and/or the presence of a G-allele at the rs10757269 loci, and then either submitting them to a classification algorithm or comparing them with a reference amount (e.g., a cut off reference amount as disclosed herein) and/or pattern of biomarkers that is associated with the particular risk level.

### Determining Stage of Risk of Major Adverse Event

In one embodiment, the present invention provides methods, kits, screens, systems and assays for determining the severity or stage or risk of having a major adverse event in a subject. Each stage of a disease will have a characteristic amount of a biomarker or relative amounts of a set of biomarkers (a pattern). The stage of a disease is determined by measuring the relevant biomarkers (e.g., beta-2-microglobulin, CRP and cystatin-C alone or in combination with other biomarkers) and/or the presence of G-allele at the rs10757269 loci and then either submitting them to a classification algorithm or comparing them with a reference amount and/or pattern of biomarkers that is associated with the particular stage, e.g., how soon the subject will likely have a major adverse event. For example, one can classify between likely to have a major adverse event within a year (e.g., a poor prognosis) or a subject likely to have an major adverse event in the next 5 years.

### Determining Decrease or Increase Risk of Having a Major Adverse Event Over a Period of Time.

In one embodiment, the present invention provides methods, kits, assays, systems and screens for determining an increase or decreased risk of having a major adverse event over a period of time in the subject. Thus, the risk of having a major adverse event can be monitored over time, and where the risk increases, it indicates disease progression (worsening) and where the risk decreases, it indicates disease regression (improvement). Over time, the amounts or relative amounts (e.g., the pattern) of the biomarkers changes (e.g., beta-2-microglobulin, CRP and cystatin-C alone or in combination with other biomarkers) are measured. For example, high beta-2-microglobulin levels, and/or high CRP, and/or high cystatin C levels and/or the presence of the G-allele at the rs10757269 loci are correlated with a risk of having a major adverse event. Therefore, the trend of these markers, either increased or decreased over time toward low-risk or non-MAE and with or without the presence of a G-allele at the rs10757269 loci, can be used to monitor the change in risk of having a major adverse event. Accordingly, this method involves measuring one or more biomarkers (e.g., beta-2-microglobulin, CRP and cystatin-C alone or in combination with other biomarkers) and/or the presence of a G-allele at the rs10757269 loci in a biological sample from the subject for at least two different time points, e.g., a first time and a second time, and comparing the change in amounts, if any. The change in the risk of an adverse is determined based on these comparisons.

### Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In some embodiments of the invention, a risk of having a major adverse event based on levels of (1) beta2-microglobulin and/or CRP and/or cystatin C in a biological sample from the subject, and/or (2) the presence of a G-allele at the rs10757269 loci, is communicated to the subject after the levels or prognosis are obtained. The prognosis or diagnosis may be communicated to the subject by the subject's treating physician. Alternatively, the prognosis or diagnosis may be sent to the subject by email or communicated to the subject by phone. A computer may be used to communicate the prognosis or diagnosis by email or phone, or via the internet using a secure gateway patient log-in service. In certain embodiments, the message containing results of the prognosis or diagnostic test may be generated and delivered automatically to the subject using a combination of computer hardware and software which will be familiar to artisans skilled in telecommunications. One example of a healthcare-oriented communications system is described in U.S. Pat. No. 6,283,761, which is incorporated herein in its entirety by reference; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Subject Management

In certain embodiments of the methods of qualifying or assessing a risk of a major adverse event, the methods further comprise managing subject treatment based on the risk of having a major adverse event. Such management includes the actions of the physician or clinician subsequent to determining the subjects risk of having a major adverse event. For example, if a physician makes a diagnosis of the subject at risk of a major adverse event, then a certain regimen of treatment may follow. A suitable regimen of treatment may include, without limitation, a supervised exercise program; control of blood pressure, sugar intake, and/or lipid levels; cessation of smoking, including any necessary counseling and nicotine replacement; and drug therapies including the administration of aspirin (with or without dipyridamole), clopidogrel, cilostazol, and/or pentoxifylline. Alternatively, a diagnosis of a risk of having a major adverse event can be followed by further testing to determine whether a patient is suffering from a specific cardiovascular disease or disorder, or whether the patient is suffering from related diseases such as coronary artery disease. Also, if the diagnostic test gives an inconclusive result on the risk of a major adverse event status, further tests may be called for.

### Generation of Classification Algorithms for Qualifying Risk of a Subject Likely to Experience a Major Adverse Event

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In some embodiments, supervised classification method is a recursive partitioning process. Recursive partitioning processes use recursive partitioning trees to classify spectra derived from unknown samples. Further details about recursive partitioning processes are provided in U.S. Pat. No. 6,675,104 (Paulse et al., “Method for analyzing mass spectra”).

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof′), U.S. Patent Application No. 2002 0193950 A1 (Gavin et al., “Method for analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for identifying a subject at risk of a major adverse event. The classification algorithms, in turn, form the base for diagnostic tests by providing diagnostic values (e.g., cut-off points) for biomarkers used singly or in combination.

### Compositions of Matter

In another aspect, this invention provides compositions of matter based on the biomarkers of this invention, e.g., the beta2-microglobulin, CRP and cystatin C.

In some embodiments, the present invention provides the biomarker of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found. The biomarkers can be isolated from biological fluids, such as urine or serum. Biomarkers may be purified using any method known in the art, including, but not limited to, mechanical separation (e.g., centrifugation), ammonium sulphate precipitation, dialysis (including size-exclusion dialysis), electrophoresis (e.g. acrylamide gel electrophoresis) size-exclusion chromatography, affinity chromatography, anion-exchange chromatography, cation-exchange chromatography, and methal-chelate chromatography. Such methods may be performed at any appropriate scale, for example, in a chromatography column, or on a biochip.

In another embodiment, this invention provides a biospecific capture reagent, optionally in purified form, that specifically binds a biomarker of this invention. In one embodiment, the biospecific capture reagent is an antibody. Such compositions are useful for detecting the biomarker in a detection assay, e.g., for diagnostics.

In another embodiment, this invention provides an article comprising a biospecific capture reagent that binds a biomarker of this invention, wherein the reagent is bound to a solid phase. For example, this invention contemplates a device comprising bead, chip, membrane, monolith or microtiter plate derivatized with the biospecific capture reagent. Such articles are useful in biomarker detection assays.

In another aspect this invention provides a composition comprising a biospecific capture reagent, such as an antibody, bound to a biomarker of this invention, the composition optionally being in purified form. Such compositions are useful for purifying the biomarker or in assays for detecting the biomarker.

In another embodiment, this invention provides an article comprising a solid substrate to which is attached an adsorbent, e.g., a chromatographic adsorbent or a biospecific capture reagent, to which is further bound a biomarker of this invention. In one embodiment, the article is a biochip or a probe for mass spectrometry, e.g., a SELDI probe. Such articles are useful for purifying the biomarker or detecting the biomarker.

### Kits for Detection of Biomarkers for Determining a Subject at Risk of Having an Major Adverse Event

In another aspect, the present invention provides kits for qualifying the risk of a major adverse event, which kits are used to detect biomarkers according to the invention. In one embodiment, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI, such as ProteinChip™ arrays. In the case of biospecific capture reagents, the kit can comprise a solid support with a reactive surface, and a container comprising the biospecific capture reagent (e.g., an antibody for beta2-microglobulin).

In some embodiments, the kits comprise probes, e.g., but not limited to, antibodies or antibody fragments which bind to the biomarkers ((e.g., beta-2-microglobulin, CRP and cystatin-C alone or in combination with other biomarkers) as disclosed herein. In some embodiments, the kits can comprise probes which can be used to detect the presence of a G-allele at the rs10757269 loci, e.g., but not limited to, allele-specific primers or allele-specific hybridization probes.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

### Determining Therapeutic Efficacy of Pharmaceutical Drug

In another embodiment, this invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of beta2-microglobulin (or CRP and/or cystatin C) changes toward a non-MAE profile, or reduced risk of major adverse event. For example, beta-2-microglobulin is increased in subjects with an increased risk of a major adverse event. Therefore, one can follow the effect of treatment (and other biomarkers) in the subject diagnosed with a risk of having a major adverse event during the course of treatment. Accordingly, this method involves measuring one or more biomarkers (e.g., beta-2-microglobulin, CRP, and/or cystatin C) in a subject receiving drug therapy, and correlating the amounts of the biomarkers (e.g., beta-2-microglobulin, CRP, and/or cystatin C) with the risk of a major adverse event in the subject, where a decrease in the risk of a major adverse event in the subject over the course of the treatment indicates that the subject is effective at decreasing the risk of a major adverse event in the subject. One embodiment of this method involves determining the levels of the biomarkers (e.g., beta-2-microglobulin, CRP, and/or cystatin C) for at least two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal (e.g., decrease), while if treatment is ineffective or alternatively, increase the risk of the subject having a major adverse event, the biomarkers will increase or elevate towards and above the threshold cut-off reference levels.

### Systems and Computer Readable Media

One aspect of the present invention relates to a system for assessing if a subject has a risk of a major adverse event, the system as shown as an exemplary example in FIG. 3 comprises: (a) a determination module configured to receive a biological sample, measure levels of a panel of biomarkers (e.g., beta-2-microglobulin, CRP, cystatin C levels), in the biological sample and to output information of the level of a panel of biomarkers (e.g., beta-2-microglobulin, CRP, cystatin C levels) in the biological sample; (b) a storage device configured to store biomarkers level output information from the determination module; (c) a comparison module adapted to receive input from the storage device and compare the data stored on the storage device with at least one reference threshold biomarker level, wherein if measured biomarker level is at least the same or higher than the reference threshold level for that biomarker, the comparison module provides information to an output module that the biological sample is associated with a subject that deviates from the reference threshold biomarker level; and (d) an output module for displaying the information to the user.

Another aspect of the present invention relates to a system for assessing if a subject has a risk of a major adverse event and/or PAD, where the system comprises: (a) a determination module configured to receive a biological sample, perform a genotyping assay to detect the presence of a G-allele at the rs10757269 loci in the biological sample and to output information of presence of a G-allele at the rs10757269 loci in the biological sample; (b) a storage device configured to store the identification of the allele at the rs10757269 loci output information from the determination module; (c) a comparison module adapted to receive input from the storage device and determine the presence of a G-allele at the rs10757269 loci, wherein if there is the presence of a G-allele at the rs10757269 loci, the comparison module provides information to an output module that the biological sample comprises a G-allele at the rs10757269; and (d) an output module for displaying the information to the user.

In all aspects of the invention, methods to determine the levels of a panel of biomarkers (e.g., beta-2 microglobulin, CRP and cystatin C) can be performed using an automated machine or system. Such machines and systems generate a report, such as displaying a report on a visible screen or a printable report which indicates the levels of a panel of biomarkers (e.g., beta-2 microglobulin, CRP and cystatin C) and/or report an increase or the same as a reference threshold level for each biomarker in the panel of biomarkers, and/or if the subject from which the sample was obtained is at risk of having a major adverse event.

Accordingly, some embodiments of the invention also provide for a machine, computer systems and computer readable media for performing the steps of (i) determining the levels of a panel of biomarkers (e.g., beta-2 microglobulin, CRP and cystatin C) and/or determining the presence of a G-allele at the rs10757269 loci, (ii) indicating or reporting whether a subject is at risk of having a major adverse event and/or PAD.

Embodiments of this aspect of the present invention are described through functional modules, which are defined by computer executable instructions recorded on computer readable media and which cause a computer to perform method steps when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

### Computer Systems:

One aspect of the present invention is a computer system that can be used to determine if a subject is likely to be at risk of having a major adverse event. In such an embodiment, a computer system is connected to a determination module and is configured to obtain output data from a determination module regarding a biological specimen, where a determination module is configured to detect the levels of a panel of biomarkers (e.g., beta-2 microglobulin, CRP and cystatin C) and/or the presence of a G-allele at the rs10757269 loci in a biological sample obtained from the subject; and where the computer system comprises (a) a storage device configured to store data output from the determination module as well as reference data; where the storage device is connected to (b) a comparison module which in one embodiment, is adapted to compare the output data stored on the storage device with stored reference data, and in alternative embodiments, adapted to compare the output data with itself, where the comparison module produces report data and is connected to (c) a display module for displaying a page of retrieved content (i.e. report data from the comparison module) for the user on a client computer, wherein the retrieved content can indicate the levels of a panel of biomarkers (e.g., beta-2 microglobulin, CRP and cystatin C), and/or the presence of a G-allele at the rs10757269 loci and/or likelihood of the subject experiencing a major adverse event and/or PAD in the future.

As an example, determination modules for determining the levels of a panel of biomarkers (e.g., beta-2 microglobulin, CRP and cystatin C) may include known systems for automated detection of proteins and biomarkers, including but not limited Mass Spectrometry systems including MALDI-TOF, or Matrix Assisted Laser Desorption Ionization—Time of Flight systems; SELDI-TOF-MS ProteinChip array profiling systems, e.g. Machines with CIPHERGEN PROTEIN BIOLOGY SYSTEM II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, COMPLETE GENECHIP® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GENE TITAN™ Instrument, GeneChip® Array Station, HT Array; an automated ELISA system (e.g. DSX® or DK® form Dynax, Chantilly, Va. or the ENEASYSTEM III®, TRITURUS®, THE MAGO® Plus); Densitometers (e.g. X-Rite-508-Spectro Densitometer®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

As an example, a determination module for determining the levels of a panel of biomarkers (e.g., beta-2 microglobulin, CRP and cystatin C) and/or the presence of a G-allele at the rs10757269 loci in the biological sample obtained from the subject may include known systems for automated protein expression level determination, including for example, but not limited to, mass spectrometry systems including Matrix Assisted Laser Desorption Ionization—Time of Flight (MALDI-TOF) systems and SELDI-TOF-MS ProteinChip array profiling systems; systems for analyzing gene expression data (see, for example, published U.S. Patent Application, Pub. No. U.S. 2003/0194711, which is incorporated herein in its entirety by reference); systems for array based expression analysis: e.g., HT array systems and cartridge array systems such as GENECHIP® AUTOLOADER, COMPLETE GENECHIP® Instrument System, GENECHIP® Fluidics Station 450, GENECHIP® Hybridization Oven 645, GENECHIP® QC Toolbox Software Kit, GENECHIP® Scanner 3000 7G plus Targeted Genotyping System, GENECHIP® Scanner 3000 7G Whole-Genome Association System, GENE TITAN™ Instrument, and GENECHIP® Array Station (each available from Affymetrix, Santa Clara, Calif.); automated ELISA systems (e.g., DSX® or DK® (available from Dynax, Chantilly, Va.) or the TRITURUS® (available from Grifols USA, Los Angeles, Calif.), The MAGO® Plus (available from Diamedix Corporation, Miami, Fla.); Densitometers (e.g. X-Rite-508-SPECTRO DENSITOMETER® (available from RP IMAGINGTm, Tucson, Ariz.), The HYRYS™ 2 HIT densitometer (available from Sebia Electrophoresis, Norcross, Ga.); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, (available from Becton Dickinson, Franklin Lakes, N.J.); and radio isotope analyzers (e.g. scintillation counters).

In some embodiments, the determination module has computer executable instructions to provide information in computer readable form. As an example, a determination module for determining the level of a biomarker protein by binding of a protein-binding molecule to a protein, for example but not limited to the binding of an anti-B2M antibody to a beta-2-microglobulin protein, or anti-CRP antibody a CRP protein, or an anti-cystatin C antibody binding to a cystatin C protein include for example but are not limited to automated immunohistochemistry apparatus, for example, robotically automated immunohistochemistry apparatus which in an automated system section the tissue or biological sample specimen, prepare slides, perform immunohistochemistry procedure and detect intensity of immunostaining, such as intensity of anti-biomarker antibody staining in the biological sample or tissue and produce output data. Examples of such automated immunohistochemistry apparatus are commercially available, for example such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In situ Hybridization (ISH).

As an example, a determination module for determining (e.g., measuring) the levels of a panel of biomarkers (e.g., beta-2 microglobulin, CRP and cystatin C) may include known systems for automated protein expression analysis including but not limited Mass Spectrometry systems including MALDI-TOF, or Matrix Assisted Laser Desorption Ionization—Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array; an automated ELISA system (e.g. DSX® or DK® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

Algorithms for identifying protein expression levels and profiles, such as the total amount of the levels of a panel of biomarkers (e.g., beta-2 microglobulin, CRP and cystatin C) available in a biological sample can include the use of optimization algorithms such as the mean variance algorithm, e.g. J MP Genomics algorithm available from JMP Software.

In some embodiments of this aspect and all other aspects of the present invention a variety of software programs and formats can be used to store the biomarker protein level information on the storage device. Any number of data processor structuring formats (e.g., text file or database) can be employed to obtain or create a medium having recorded thereon the sequence information or expression level information.

### Storage Module

In some embodiments, the levels of a panel of biomarkers (e.g., beta-2 microglobulin, CRP and cystatin C) and/or the presence of a G-allele at the rs10757269 loci as determined in the determination module can be read by the storage device. As used herein the “storage device” is intended to include any suitable computing or processing apparatus or other device configured or adapted for storing data or information. Examples of electronic apparatus suitable for use with the present invention include stand-alone computing apparatus; communications networks, including local area networks (LAN), wide area networks (WAN), Internet, Intranet, and Extranet; and local and distributed processing systems. Storage devices also include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon sequence information or expression level information. The data are typically provided in digital form that can be transmitted and read electronically, e.g., via the Internet, on diskette, or any other mode of electronic or non-electronic communication.

Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, other types of volatile and non-volatile memory, any other medium which can be used to store the desired information and which can accessed by a computer, and any suitable combination of the foregoing. The computer readable media does not encompass a data signal or a carrier wave, preferably the computer readable medium is of physical form.

In some embodiments of this aspect and all other aspects of the present invention, a computer readable media can be any available media that can be accessed by a computer. By way of example, and not a limitation, computer readable media may comprise computer storage media and communication media.

As used herein, “stored” refers to a process for encoding information on the storage device. Those skilled in the art can readily adopt any of the presently known methods for recording information on known media to generate manufactures comprising the sequence information or expression level information.

In some embodiments of this aspect and all other aspects of the present invention a variety of software programs and formats can be used to store the phosphorylation information or expression level information on the storage device. Any number of data processor structuring formats (e.g., text file or database) can be employed to obtain or create a medium having recorded thereon the sequence information or expression level information.

In some embodiments of this aspect and all other aspects of the present invention, the reference data stored in the storage device to be read by the comparison module is sequence information data obtained from a control biological sample of the same type as the biological sample to be tested. Alternatively, the reference data are a database, e.g., a part of the entire genome sequence of an organism, or a protein family of sequences, or an expression level profile (RNA, protein or peptide). In one embodiment the reference data are sequence information or expression level profiles that are indicative of a specific disease or disorder.

In some embodiments of this aspect and all other aspects of the present invention, the reference data are electronically or digitally recorded and annotated from databases including, but not limited to GenBank (NCBI) protein and DNA databases such as genome, ESTs, SNPS, Traces, Celara, Ventor Reads, Watson reads, HGTS, etc.; Swiss Institute of Bioinformatics databases, such as ENZYME, PROSITE, SWISS-2DPAGE, Swiss-Prot and TrEMBL databases; the Melanie software package or the ExPASy WWW server, etc., the SWISS-MODEL, Swiss-Shop and other network-based computational tools; the Comprehensive Microbial Resource database (The institute of Genomic Research). The resulting information can be stored in a relational data base that may be employed to determine homologies between the reference data or genes or proteins within and among genomes.

### Comparison Module

By providing the levels of a panel of biomarkers (e.g., beta-2 microglobulin, CRP and cystatin C) and/or presence of a G-allele at the rs10757269 loci in readable form in the comparison module, it can be used to compare with the reference threshold levels of each biomarker and/or other alleles at the rs10757269 within the storage device. The comparison made in computer-readable form provides computer readable content which can be processed by a variety of means. The content can be retrieved from the comparison module, the retrieved content.

In some embodiments of this aspect and all other aspects of the present invention, the “comparison module” can use a variety of available software programs and formats for the comparison operative to compare sequence information determined in the determination module to reference data. In one embodiment, the comparison module is configured to use pattern recognition techniques to compare sequence information from one or more entries to one or more reference data patterns. The comparison module may be configured using existing commercially-available or freely-available software for comparing patterns, and may be optimized for particular data comparisons that are conducted. The comparison module provides computer readable information related to the sequence information that can include, for example, the presence of a G-allele at the rs10757269 loci, or detection of the presence or absence of a sequence (e.g., detection of a G-allele at position 27 of SEQ. ID NO: 1, information regarding distinct alleles, or omission or repetition of sequences); determination of the concentration of a sequence in the sample (e.g. amino acid sequence/protein expression levels, or nucleotide (RNA or DNA) expression levels), or determination of an expression profile.

In one embodiment, the comparison module permits the comparison of the levels of a panel of biomarkers (e.g., beta-2 microglobulin, CRP and cystatin C) and/or the presence of a G-allele at the rs10757269 loci from the output data of the determination module with reference threshold level data for each biomarker or the rs10757269 loci.

In one embodiment, the comparison module performs comparisons with mass-spectrometry spectra, for example comparisons of peptide fragment sequence information can be carried out using spectra processed in MATLB with script called “Qcealign” (see for example WO2007/022248, herein incorporated by reference) and “Qpeaks” (Spectrum Square Associates, Ithaca, N.Y.), or Ciphergen Peaks 2.1™ software. The processed spectra can then be aligned using alignment algorithms that align sample data to the control data using minimum entropy algorithm by taking baseline corrected data (see for example WO2007/022248, herein incorporated by reference). The retrieved content can be further processed by calculating ratios

In one embodiment, the comparison module compares the protein phosphorylation profiles. In one embodiment, the comparison module compares gene expression profiles. For example, detection of gene expression profiles can be determined using Affymetrix Microarray Suite software version 5.0 (MAS 5.0) to analyze the relative abundance of a gene or genes on the basis of the intensity of the signal from probe sets and the MAS 5.0 data files can be transferred into a database and analyzed with Microsoft Excel and GeneSpring 6.0 software (Silicon genetics). The detection algorithm of MAS 5.0 software can be used to obtain a comprehensive overview of how many transcripts are detected in given samples and allows a comparative analysis of 2 or more microarray data sets.

Any available comparison software can be used, including but not limited to, the Ciphergen Express (CE) and Biomarker Patterns Software (BPS) package, Ciphergen Biosystems, Inc., CA, USA. Comparative analysis can be done with protein chip system software (e.g. The Proteinchip suite for Bio-Rad Laboratories).

In one embodiment, computational algorithms such as expectation-maximization (EM), subtraction and PHASE are used in methods for statistical estimation of haplotypes (see, e.g., Clark, A. G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

In some embodiments of this aspect and all other aspects of the present invention, the comparison module, or any other module of the invention, may include an operating system (e.g., UNIX) on which runs a relational database management system, a World Wide Web application, and a World Wide Web server. World Wide Web application includes the executable code necessary for generation of database language statements [e.g., Standard Query Language (SQL) statements]. Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In some embodiments of this aspect and all other aspects of the present invention, a computer-readable data embodied on one or more computer-readable media may define instructions, for example, as part of one or more programs, that, as a result of being executed by a computer, instruct the computer to perform one or more of the functions described herein (e.g., in relation to computer system, or computer readable medium), and/or various embodiments, variations and combinations thereof. Such instructions may be written in any of a plurality of programming languages, for example, Java, J, Visual Basic, C, C#, or C++, Fortran, Pascal, Eiffel, Basic, COBOL, etc., or any of a variety of combinations thereof. The computer-readable media on which such instructions are embodied may reside on one or more of the components of either of computer system [or machine], or computer readable medium described herein, may be distributed across one or more of such components, and may be in transition there between.

In some embodiments of this aspect and all other aspects of the present invention, a computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement aspects of the present invention. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Instructions can be provided to the computer systems 150 which refers to a number of computer-implemented steps for processing information in the system. Instructions can be implemented in software, firmware or hardware and include any type of programmed step undertaken by modules of the electronic financing system. The computer system 150 can be connected to a local network. One example of the Local Area Network may be a corporate computing network, including access to the Internet, to which computers and computing devices comprising the financing system are connected. In one embodiment, the LAN conforms to the Transmission Control Protocol/Internet Protocol (TCP/IP) industry standard. Transmission Control Protocol Transmission Control Protocol (TCP) is a transport layer protocol used to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data re-transmissions, and multiplexing multiple connections through a single network connection. Finally, TCP is responsible for encapsulating information into a datagram structure.

In alternative embodiments, the LAN may conform to other network standards, including, but not limited to, the International Standards Organization's Open Systems Interconnection, IBM's SNA, Novell's Netware, and Banyan VINES. The computer system may comprise a microprocessor. A microprocessor may be any conventional general purpose single-or multi-chip microprocessor such as a PentiumW processor, a PentiumX Pro processor, a 8051 processor, a MISS, processor, a Power PC′processor, or an ALPHAZ processor. In addition, the microprocessor may be any conventional special purpose microprocessor such as a digital signal processor or a graphics processor. The microprocessor typically has conventional address lines, conventional data lines, and one or more conventional control lines.

In some embodiments, the computer system 150 as described herein can include any type of electronically connected group of computers including, for instance, the following networks: Internet, Intranet, Local Area Networks (LAN) or Wide Area Networks (WAN). In addition, the connectivity to the network may be, for example, remote modem, Ethernet (IEEE 802.3), Token Ring (IEEE 802.5), Fiber Distributed Datalink Interface (FDDI) or Asynchronous Transfer Mode (ATM). Note that computing devices may be desktop, server, portable, hand-held, set-top, or any other desired type of configuration. As used herein, an Internet includes network variations such as public internet, a private internet, a secure internet, a private network, a public network, a value-added network, an intranet, and the like.

The computer systems and comparison module can use a variety of operating Systems. For example the computer system 150 can be used in connection with various operating systems such as: UNIX, Disk Operating System (DOS), OS/2, Windows 3, X, Windows 95, Windows 98, and Windows NT. The computer system 150 as described herein can be programmed in any programming language, for example the system may be written in any programming language such as C, C++, BASIC, Pascal, Java, and FORTRAN and ran under the well-known operating system. C, C++, BASIC, Pascal, Java, and FORTRAN are industry standard programming languages for which many commercial compilers can be used to create executable code.

In one embodiment of the invention, the computer system can comprise a pattern comparison software can be used to determine whether patterns of the levels of a panel of biomarkers (e.g., beta-2 microglobulin, CRP and cystatin C) are indicative of a subject being at risk of having a major adverse event.

In some embodiments of this aspect and all other aspects of the present invention, a comparison module provides computer readable data that can be processed in computer readable form by predefined criteria, or criteria defined by a user, to provide a retrieved content that may be stored and output as requested by a user using a display module.

In some embodiments of this aspect and all other aspects of the present invention, the retrieved content can be the identification of the levels of a panel of biomarkers (e.g., beta-2 microglobulin, CRP and cystatin C), and/or if the levels of each biomarker are at the same level, or higher than the reference threshold level for each biomarker respectively. In another embodiment, the retrieved content is a positive indicator that the biological sample is a risk of having a major adverse event.

### Display Module

In some embodiments of this aspect and all other aspects of the present invention, a page of the retrieved content which is the report data from the comparison module is displayed on a computer monitor 120. In one embodiment of the invention, a page of the retrieved content is displayed through printable media 130 and 140. The display module 120 can be any computer adapted for display of computer readable information to a user, non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD), or any other type of processor. Other displays modules include; speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc.

In some embodiments of this aspect and all other aspects of the present invention, a World Wide Web browser is used for providing a user interface for display of the retrieved content. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars, etc. conventionally employed in graphical user interfaces. The requests so formulated with the user's Web browser are transmitted to a Web application which formats them to produce a query that can be employed to extract the pertinent information related to the levels of a panel of biomarkers (e.g., beta-2 microglobulin, CRP and cystatin C) and/or the presence of a G-allele at the rs10757269 loci, the retrieved content, e.g. display of an indication of the levels of a panel of biomarkers (e.g., beta-2 microglobulin, CRP and cystatin C); and/or the presence or absence of a G-allele at the rs10757269 loci, and/or display of expression levels of an amino acid sequence (protein); display of nucleotide (RNA or DNA) expression levels; or display of expression, SNP, or mutation profiles, or haplotypes. In one embodiment, the sequence information of the reference sample data is also displayed.

The display module 110 also displays whether the retrieved content is indicative of the subject being at risk of experiencing a major adverse event, e.g. whether the levels of a panel of biomarkers (e.g., beta-2 microglobulin, CRP and cystatin C) in the biological sample from the subject were at the same level or higher than the reference threshold level for each biomarker as compared the control subject and/or the presence of a G-allele at the rs10757269 loci. In one embodiment, the retrieved content displayed is a positive signal identifying that the levels of a panel of biomarkers (e.g., beta-2 microglobulin, CRP and cystatin C) were at the same level or higher than the reference threshold level for each biomarker (or conversely a negative signal if the levels of biomarkers were below the reference threshold level for each biomarker respectively), where a positive signal indicates the subject has a risk of having a major adverse event in the future.

In one embodiment, the retrieved content displayed is a positive signal identifying that the presence of a G-allele at the rs10757269 loci (or conversely a negative signal if there is an absence of a G-allele at the rs10757269 loci), where a positive signal indicates the subject has a risk of having a major adverse event and/or PAD in the future.

### Application of the Methods, Kits, Machines, Computer Systems, Computer Readable Media:

In the research context, embodiments of the invention may provide a method for drug screening and reporting of drug effects in preclinical and clinical trials. The inventive methods can be used to identify which subjects are likely to be responsive to treatment to reduce risk of a major adverse event, assess the effectiveness of a therapy or regimen to reduce a subjects likelihood of having a major adverse event, improve the quality and reduce costs of clinical trials, discover the subset of positive responders to a particular class of therapy or treatment for reducing incidence of a major adverse event (i.e. stratifying patient populations), improve therapeutic success rates, and/or reduce sample sizes, trial duration and costs of clinical trials.

In the health care context, embodiments of the invention may provide a service to physicians that will enable the physicians to tailor optimal personalized patient therapies. For example, a biological sample taken from a subject can be sent by the pathologist and/or clinical oncologist to a laboratory facility, for example, one such lab is operated by THERANOSTICS HEALTH™, LLC. The laboratory may analyze the levels of the panel of biomarkers (e.g., beta-2 microglobulin, CRP and cystain-c) and/or the presence of a G-allele at the rs10757269 loci in a biological sample from a subject and provide a report to the physician or health care provider. The laboratory may provide the treating pathologist or clinician with a report indicating if the subject from which the biological sample was taken is likely to be at risk of having a major adverse event, and optionally provide a listing of suitable therapies and regimens which can be recommended to a subject identified as being at risk of having a major adverse event. This may enable a physician or clinician to tailor therapy to the individual subject's tumor or other disorder, prescribe the right therapy to the right patient at right time, provide a higher treatment success rate, spare the patient unnecessary toxicity and side effects, reduce the cost to patients and insurers of unnecessary or dangerous ineffective medication, and improve patient quality of life, eventually making cancer a managed disease, with follow up assays as appropriate. Physicians can use the reported information to tailor optimal personalized patient therapies instead of the current “trial and error” or “one size fits all” methods used to prescribe chemotherapy under current systems. The inventive methods may establish a system of personalized medicine.

### Use of Biomarkers for Diagnosing a Major Adverse Event in Screening Assays

The methods of the present invention have other applications as well. For example, the biomarkers (e.g., beta-2-microglobulin, CRP, and/or cystatin C) can be used to screen for compounds that modulate the expression of the biomarkers in vitro or in vivo, which compounds in turn may be useful in treating or preventing a major adverse event in patients. In another example, the biomarkers can be used to monitor the response to treatments for decreasing the risk of a major adverse event in the subject. In yet another example, the biomarkers (e.g., beta-2-microglobulin, CRP, and/or cystatin C) can be used in heredity studies to determine if the subject is at risk for having a major adverse event, as well a genetic susceptibility of subjects with a high risk of a having a major adverse event.

Compounds suitable for therapeutic testing may be screened initially by identifying compounds which interact with beta-2-microglobulin and CRP, and/or cystatin C, one or more additional biomarkers. By way of example, screening might include recombinantly expressing a biomarker, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of beta-2-microglobulin, CRP, and/or cystatin C, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of beta-2-microglobulin, CRP, cystatin C or another one or more of the biomarkers herein may also be measured. For example, the self-assembly of a multi-protein complex which includes beta-2-microglobulin may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of beta-2-microglobulin, CRP, and/or cystatin C may be administered to patients who are suffering from or are at risk of having a major adverse event. For example, the administration of a test compound which increases the activity of a particular biomarker (e.g., beta-2-microglobulin, CRP, and/or cystatin C) may decrease the risk of the subject having a major adverse event if the activity of the particular biomarker in vivo prevents the accumulation of proteins which can contribute to a major adverse event. Conversely, the administration of a test compound which decreases the activity of a particular biomarker (e.g., beta-2-microglobulin, CRP, and/or cystatin C) may decrease the risk of having a major adverse event in a patient if the increased activity of the biomarker is responsible, at least in part, for the onset of the major adverse event.

In an additional aspect, the invention provides a method for identifying compounds useful for reducing the risk of having a major adverse event by using modified forms of beta-2-microglobulin, CRP, or cystatin C. For example, in one embodiment, cell extracts or expression libraries may be screened for compounds which catalyze the cleavage of full-length beta-2-microglobulin to form truncated forms of beta-2-microglobulin. In one embodiment of such a screening assay, cleavage of beta-2-microglobulin may be detected by attaching a fluorophore to beta-2-microglobulin which remains quenched when beta-2-microglobulin is uncleaved but which fluoresces when the protein is cleaved. Alternatively, a version of full-length beta-2-microglobulin modified so as to render the amide bond between amino acids x and y uncleavable may be used to selectively bind or “trap” the cellular protease which cleaves full-length beta-2-microglobulin at that site in vivo. Methods for screening and identifying proteases and their targets are well-documented in the scientific literature, e.g., in Lopez-Ottin et al. (Nature Reviews, 3:509-519 (2002)).

In yet another embodiment, the invention provides a method for treating or reducing the risk of having a major adverse event which is associated with the increased levels of truncated beta-2-microglobulin. For example, after one or more proteins have been identified which cleave full-length beta-2-microglobulin, combinatorial libraries may be screened for compounds which inhibit the cleavage activity of the identified proteins. Methods of screening chemical libraries for such compounds are well-known in art. See, e.g., Lopez-Otin et al. (2002). Alternatively, inhibitory compounds may be intelligently designed based on the structure of beta-2-microglobulin.

Full-length beta-2-microglobulin is believed to be involved in regulation of the body's iron stores, as well as in hereditary hemochromatosis, chronic renal insufficiency, and renal anemia. Beta-2-microglobulin expression is also induced as part of the body's immune response via the interleukin cascade. Because beta-2-microglobulin is highly processed from its pre-pro and pro-forms, it is likely that there are proteases which target and cleave it. Therefore, in a further embodiment, the invention provides methods for identifying compounds which increase the affinity of truncated beta-2-microglobulin for its target proteases. For example, compounds may be screened for their ability to cleave beta-2-microglobulin. Test compounds capable of modulating the cleavage of beta-2-microglobulin or the activity of molecules which interact with beta-2-microglobulin may then be tested in vivo for their ability to slow the occurrence or decrease the risk of a major adverse event in a subject.

At the clinical level, screening a test compound includes obtaining samples from test subjects before and after the subjects have been exposed to a test compound. The levels in the samples of one or more of the biomarkers (e.g., beta-2-microglobulin, CRP, and/or cystatin C) may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers (e.g., beta-2-microglobulin, CRP, and/or cystatin C) may be measured directly by Western blot using radio-or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers (e.g., beta-2-microglobulin, CRP, and/or cystatin C) may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with a high risk of a major adverse event, test compounds will be screened for their ability to slow or reduce the occurrence of a major adverse event.

An isolated biomarker can also be used for the development of diagnostic and other tissue evaluating kits and assays to monitor the level of the biomarkers (e.g., beta-2 microglobulin, CRP and cystatin C) in a tissue or fluid sample. For example, the kit may include antibodies or other specific binding proteins which bind specifically to one or more biomarkers (e.g., beta-2 microglobulin, CRP and cystatin C) and which permit the presence and/or amount of the one or more biomarkers to be detected and/or quantified in a tissue or fluid sample.

Suitable kits for detecting one or more biomarkers (e.g., beta-2 microglobulin, CRP and cystatin C) are contemplated to include, but are not limited to, a receptacle or other means for capturing a sample to be evaluated and a means for detecting the presence and/or amount in the sample of one or more of the biomarkers (e.g., beta-2 microglobulin, CRP and cystatin C) described herein. Means for detecting in one embodiment includes, but is not limited to, one or more antibodies specific for these biomarkers (e.g., beta-2 microglobulin, CRP and cystatin C) and means for detecting the binding of the antibodies to these biomarkers by, for example, a standard sandwich immunoassay as described herein. Where the presence of a biomarker located within a cell is to be detected (e.g., as from a tissue sample) the kit also may comprise means for disrupting the cell structure so as to expose intracellular components.

The biomarkers (e.g., beta-2 microglobulin, CRP and cystatin C) of the present teachings may include nucleic acids of a particular sequence. One or more of the biomarkers (e.g., beta-2 microglobulin, CRP and cystatin C) may be detected and/or quantified by determining an amount or absolute concentration of the biomarker (e.g., beta-2 microglobulin, CRP and cystatin C) nucleic acid in a sample, using, for example, Real-Time Quantitative PCR (RT-PCR) and comparing the measured amount to a standard to determine a relative concentration of the biomarker (e.g., beta-2 microglobulin, CRP and cystatin C) nucleic acid in a sample. RT-PCR effectively measures the amount of a biomarker nucleic acid (e.g., mRNA levels for beta-2 microglobulin, CRP and cystatin C) resulting from PCR. A positive result represents a measured amount of the biomarker nucleic acid that is different than the amount of the biomarker (e.g., beta-2 microglobulin, CRP and cystatin C) from a standard, or a relative concentration having a value above or below zero.

Primers can be developed that are complementary to the nucleic acid sequence of a particular nucleic acid biomarker. These primers direct a polymerase to copy and amplify that particular nucleic acid. RT-PCR detects the accumulation of the amplified nucleic acid biomarker during the reaction. During the exponential phase of the PCR reaction, the accumulating nucleic acid of the biomarker can be measured. A calibration standard having a known concentration of nucleic acid can be used to prepare a standard curve from which the quantity of the nucleic acid biomarker (e.g., beta-2 microglobulin, CRP and cystatin C) in the tested sample can be extrapolated. Once the amount or absolute concentration of a nucleic acid of the biomarker in a sample is known, it can be compared to the amount of the nucleic acid biomarker from a standard to determine a relative concentration of a nucleic acid biomarker in a sample. The standard for classification of major adverse cardiovascular or cerebrovascular event subjects can be determined by empirical means. For example, the amount can be determined by amplifying the nucleic acid biomarker in a sample from a population of one or more known normal individuals and quantitatively analyzing the amount of a nucleic acid biomarker in the population.

Also, additional forms of chemical analysis of a sample can be performed. For example, quantitative tests can be carried out that indicate the amounts or absolute concentrations of each biomarker (e.g., beta-2 microglobulin, CRP and cystatin C) in a sample. A colorimetric assay is a quantitative chemical analysis measuring color intensity produced by reacting a sample with a reactant as a proxy for the amount of the assayed biomarker (e.g., beta-2 microglobulin, CRP and cystatin C) in a sample. Reagents can be provided that, when reacted with any analyte, produce a color in the assay sample. The intensity of that color can be dependent on the amount of the biomarker (e.g., beta-2 microglobulin, CRP and cystatin C) in the sample. By comparison of the intensity with a calibrated color card and/or standard, the amount of the biomarker in the sample can be determined. This amount can then be compared with the amount of the biomarker (e.g., beta-2 microglobulin, CRP and cystatin C) from a standard (such as from a known normal person) to determine a relative concentration of the biomarker (e.g., beta-2 microglobulin, CRP and cystatin C) in a sample.

Additionally, urinalysis can be used to determine the amount or absolute concentration of the biomarkers (e.g., beta-2 microglobulin, CRP and cystatin C) in a urine sample. Urine samples are tested with a variety of different instruments and techniques. Some tests use dipsticks, which are thin strips of plastic that change color in the presence of specific substances. Dipsticks could be used to measure the amount of the biomarkers (e.g., beta-2 microglobulin, CRP and cystatin C).

Not only does comparing the absolute level or concentration of each of at least three biomarkers (e.g., beta-2 microglobulin, CRP and cystatin C) to the level of each of the biomarkers from a standard level (e.g., a reference threshold level) to determine a relative concentration of each of the biomarker allow for diagnosis of having or being at risk of having a major adverse event, but this same comparison methodology can be adapted to other uses. For example, the biomarkers (e.g., beta-2 microglobulin, CRP and cystatin C) can be used to screen candidate drugs for treating a major adverse event. In this instance, treatment with candidate drugs can be monitored by monitoring the level of the biomarkers (e.g., beta-2 microglobulin, CRP and cystatin C). To the extent the absolute concentration of the biomarkers (e.g., beta-2 microglobulin, CRP and cystatin C) returned to the standard level from the diseased level, whereby the relative concentration approaches zero, efficacy can be determined. Moreover, with any drug that has already been found effective to treat a major adverse event, it may be that certain subjects may be responders and some may be non-responders. Accordingly, the biomarkers (e.g., beta-2 microglobulin, CRP and cystatin C) could be monitored during treatment to determine if the drug is effective by determining if the absolute level or concentration of the biomarkers (e.g., beta-2 microglobulin, CRP and cystatin C) return to the standard level, whereby the relative concentration approaches zero. Of course, there may not be any existing, known population of responders and non-responders, so that the efficacy of drug treatment on any major adverse event subject can be monitored over time. To the extent it is not efficacious, its use can be discontinued and another drug supplied in its place.

Moreover, determining a relative concentration by comparing the absolute level or concentration of each of the biomarkers (e.g., beta-2 microglobulin, CRP and cystatin C) to the level of each of the biomarkers to a reference threshold level can be done as a preventative screening measure and not just when an adverse is observed (i.e., after the disease may have progressed). For example, assuming no evidence of an adverse event, subjects could be monitored after a certain age and at predetermined intervals in order to obtain a diagnosis of having or being at risk of having a major adverse event at the earliest possible time. To the extent the screen is positive, a medical professional might recommend further monitoring for disease progression, and/or the medical professional might begin treatments with a drug or other therapy.

The results of the analysis, including, for example, the amount or absolute concentration of each of the biomarkers (e.g., beta-2 microglobulin, CRP and cystatin C), the relative concentration of each of the biomarkers (e.g., beta-2 microglobulin, CRP and cystatin C) to a reference threshold level or standard, and/or a likelihood of having or being at risk of having a major adverse event, can be displayed or outputted to a user interface device, a computer readable storage medium, or a local or remote computer system. Displaying or outputting a result or diagnosis means that the results of any of the foregoing analyses are communicated to a user using any medium, such as for example, orally, writing, visual display, etc., computer readable medium or computer system. It will be clear to one skilled in the art that outputting the result is not limited to outputting to a user or a linked external component(s), such as a computer system or computer memory, but may alternatively or additionally be outputted to internal components, such as any computer readable medium. Computer readable media may include, but are not limited to hard drives, floppy disks, CD-ROMs, DVDs, and DATs. Computer readable media does not include carrier waves or other wave forms for data transmission. It will be clear to one skilled in the art that the various sample evaluation and diagnosis methods disclosed and claimed herein, can, but need not be, computer-implemented, and that, for example, the displaying or outputting step can be done by, for example, by communicating to a person orally or in writing (e.g., in handwriting).

Moreover, the biomarkers (e.g., beta-2 microglobulin, CRP and cystatin C) can be used to validate animal models of major adverse events. For example, in any particular model, a sample could be analyzed to determine if levels of the biomarkers (e.g., beta-2 microglobulin, CRP and cystatin C) in the animal are the same as the levels of the biomarkers (e.g., beta-2 microglobulin, CRP and cystatin C) in a known major adverse event subject. This would validate the model, for example, to test candidate drugs in the manner described above.

### Kits

In some embodiments, the present invention further includes a kit for use in a method of measuring the amount of the panel of biomarkers (e.g., beta-2 microglobulin, CRP and cystatin C) in a biological sample, where the kit comprises a binding partner, as described above, in an assay-compatible format, for interaction with the panel of biomarkers (e.g., beta-2 microglobulin, CRP and cystatin C) present in the biological sample. Thus, in some embodiments, it is contemplated within the invention to use an antibody chip or array of chips, capable of measuring the levels of the biomarkers (e.g., beta-2 microglobulin, CRP and cystatin C).

In some embodiments, a kit for use in a method or system for measuring the amount of the panel of biomarkers (e.g., beta-2 microglobulin, CRP and cystatin C) in a biological sample from the subject is an immunoassay, for example but not limited to immunofluorescent assay, ELISA, chemiluminescent assay, and in some embodiments, the kit can optionally include instructions for measuring biomarker levels.

In some embodiment, a kit can comprise a reference sample, e.g., a control reference sample from a healthy subject (e.g., a negative control), and in some embodiments, a positive control sample (e.g., obtained from a subject with levels of the biomarkers at or above the reference threshold levels for each biomarker).

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

The present invention may be as defined in any one of the following numbered paragraphs.

1. An assay to determine if a subject is at risk of having a major adverse event, the assay comprising:

contacting a biological sample obtained from the subject with at least one probe to detect the levels of at least three biomarkers selected from beta-2 microglobulin, C-reactive protein (CRP) and cystatin C; measuring the levels of at least three biomarkers selected from beta-2 microglobulin, C-reactive protein (CRP) and cystatin C;

wherein the level of beta-2 microglobulin, C-reactive protein (CRP) and cystatin C above a threshold reference level for each of beta-2 microglobulin, C-reactive protein (CRP) and cystatin C identifies a subject who would be predicted to be at risk of having a major adverse event.

2. The assay of paragraph 1, wherein the probe comprises a detectable label or means of generating a detectable signal.
3. An assay comprising:
a. measuring the levels of antibodies that are reactive to at least three biomarkers selected from beta-2 microglobulin, C-reactive protein (CRP), and cystatin C in a biological sample obtained from a subject who has a body mass index (BMI) of 25 or greater for determining the likelihood of the subject having a major adverse event; and
b. comparing the level of the antibodies of the least three biomarkers in the biological sample with a reference antibody level for each of beta-2 microglobulin, C-reactive protein (CRP) and cystatin C, wherein a detectable increase of each antibody for each biomarker in the biological sample above the reference antibody level indicates the likelihood of the subject at risk of having a major adverse event.
   4. The assay of any of paragraphs 1-2, wherein the probe is an antibody, antibody binding fragment or protein binding molecule.
   5. The assay of any of paragraphs 1, 2 or 4, wherein the antibody is an antibody binding fragment or protein binding molecule.
   6. The assay of any of paragraphs 1 to 5, wherein the level of beta-2-microglobulin at or above 1.88 mg/1 threshold reference level indicates that the subject is predicted to be at risk of having a major adverse event.
   7. The assay of any of paragraphs 1 to 5, wherein the level of CRP at or above 1.60 mg/l threshold reference level indicates that the subject is predicted to be at risk of having a major adverse event.
   8. The assay of any of paragraphs 1 to 5, wherein the level of cystatin C at or above 0.72 mg/l threshold reference level indicates that the subject is predicted to be at risk of having a major adverse event.
   9. The assay of any of paragraphs 1 to 8, wherein the subject is determined to have a major adverse event in the next 12 months or earlier.
   10. The assay of any of paragraphs 1 to 9, wherein the major adverse event is stroke, heart attack or death.
   11. The assay of any of paragraphs 1 to 9, wherein the major adverse event is a major adverse cardiovascular or cerebrovascular event (MACCE).
   12. The assay of paragraph 11, wherein the MACCE is selected from the group consisting of: recurrence of an initial cardiac event, angina, decompensation of heart failure, admission for cardiovascular disease (CVD), mortality due to CVD, and transplant.
   13. The assay of any of paragraphs 1 to 12, wherein additional biomarkers can be measured, selected from the group consisting of CD40, fibrinogen, IL-3, IL-8, SGOT and von Willebrand factor.
   14. The assay of any of paragraphs 1 to 13, wherein the biological sample is a blood-based sample or a urine sample.
   15. The assay of paragraph 14, wherein the blood based sample is a serum, plasma or blood sample.
   16. The assay of paragraphs 14 or 15, wherein the blood-based sample or urine sample is obtained from a subject who has fasted.
   17. The assay of any of paragraphs 1 to 16, wherein the subject is a human subject.
   18. The assay of any of paragraphs 1 to 17, wherein the subject has been diagnosed with heart failure.
   19. The assay of any of paragraphs 1 to 18, wherein the subject has a body mass index (BMI) of 25 to 29, a BMI of greater or equal to 30.
   20. The assay of any of paragraphs 1 to 19, wherein a decision to discharge a subject or to continue treating a subject in an inpatient basis is made in part on the results of the assay.
   21. The assay of any of paragraphs 1 to 20, wherein the biological sample is obtained from a subject that has been hospitalized after an acute cardiac event.
   22. The assay of any of paragraphs 1 to 21, wherein the subject has a pulmonary disorder or a liver disorder.
   23. The assay of any of paragraphs 1 to 22, wherein the antibody or probes are deposited or immobilized on a solid support.
   24. The assay of any of paragraphs 1 to 23, wherein the assay is an immunoassay.
   25. The assay of paragraph 24, wherein the immunoassay is an ELISA.
   26. The assay of paragraph 23, wherein the support is in the format of a dipstick, a test strip, a latex bead, a microsphere or a multi-plate.
   27. The assay of paragraph 26, wherein the antibody is detected by a detection antibody comprising a detectable label or a means of generating a detectable signal.
   28. The assay of any of paragraphs 1 to 27, wherein the subject is a Caucasian subject.
   29. The assay of any of paragraphs 1 to 27, wherein the subject is an African-American, Black, Hispanic, an Asian-American or an Asian subject.
   30. The assay of any of paragraphs 1 to 27, wherein the subject is of Asian-Indian, Pakistani, Middle Eastern or Pacific Islander ethnicity.
   31. An assay to determine if the subject is at risk of a major cardiac event (MAE), the comprising:
a. subjecting a biological sample obtained from a subject with a Body Mass Index (BMI) of 25 or greater to at least one genotyping assay that determines the genotype of the allele at the rs10757269 loci;
b. determining the genotype of the allele at the rs10757269 loci; and
c. selecting a treatment regimen for the subject where the subject has at least one G-allele at the rs10757269 loci and is at risk of a major cardiac event, and not selecting the treatment regimen for the subject where the subject does not have at least one G-allele at the rs10757269 loci.
   32. An assay to determine if the subject is at risk of peripheral artery disease (PAD), the comprising:
a. subjecting a biological sample obtained from a subject with a Body Mass Index (BMI) of 25 or greater to at least one genotyping assay that determines the genotype of the allele at the rs10757269 loci;
b. determining the genotype of the allele at the rs10757269 loci; and
c. selecting a treatment regimen for the subject where the subject has at least one G-allele at the rs10757269 loci and is at risk of PAD, and not selecting the treatment regimen for the subject where the subject does not have at least one G-allele at the rs10757269 loci.
   33. The assay of paragraphs 31 and 32, wherein the subject has a genotype of G/A or G/G at the rs10757269 loci.
   34. The assay of any of paragraph 31 to 33, wherein the treatment regimen is selected from any of the combination of: healthy diet, increased exercise, increased weight loss, medication to decrease blood pressure, and aspirin.
   35. The assay of any of paragraph 31 to 34, wherein the subject who has at least one G-allele at the rs10757269 loci is determined to have a major adverse event in the next 12 months or earlier.
   36. The assay of any of paragraph 31 to 35, wherein the major adverse event is stroke, heart attack or death.
   37. The assay of any of paragraph 31 to 36, wherein the major adverse event is a major adverse cardiovascular or cerebrovascular event (MACCE).
   38. The assay of paragraph 37, wherein the MACCE is selected from the group consisting of: recurrence of an initial cardiac event, angina, decompensation of heart failure, admission for cardiovascular disease (CVD), mortality due to CVD, and transplant.
   39. The assay of any of paragraph 31 to 36, wherein the biological sample is a blood-based sample or a urine sample.
   40. The assay of paragraph 39, wherein the blood based sample is a serum, plasma or blood sample.
   41. The assay of any of paragraph 31 to 40, wherein the subject is a human subject.
   42. The assay of any of paragraph 31 to 41, wherein the subject has been diagnosed with heart failure.
   43. The assay of any of paragraph 31 to 42, wherein the subject has a body mass index (BMI) of 25 to 29, or a BMI of greater or equal to 30.
   44. The assay of any of paragraph 31 to 43, wherein the biological sample is obtained from a subject that has been hospitalized after an acute cardiac event.
   45. The assay of any of paragraph 31 to 44, wherein the subject has a pulmonary disorder or a liver disorder.
   46. The assay of any of paragraphs 31 to 45, wherein the genotyping assay is selected from any or a combination in the group consisting of: PCR-based assays, RT-PCR, nucleic acid hybridization, sequence analysis, TaqMan SNP genotyping probes, microarrays, direct or indirect sequencing, restriction site analysis, hybridization based genotyping assays, gel migration assays, antibodies assays, fluorescent polarization, mass spectroscopy, allele-specific PCR, single-strand conformational polymorphism (SSCP) analysis, heteroduplex analysis, oligonucleotide ligation, PCR-RFLP, allele-specific amplification (ASA), single-molecule dilution (SMD), coupled amplification and sequencing (CAS), Restriction enzyme analysis, restriction fragment length polymorphism (RFLP), ligation based assays, single base extension (or minisequencing), MALDI-TOF, and homogenous assays.
   47. The assay of any of paragraphs 31 to 46, wherein the genotyping assay detects a G-allele at position 27 of SEQ ID NO: 1, or a C-allele in the complementary nucleic acid sequence of SEQ ID NO: 1.
   48. The assay of any of paragraphs 31 to 47, wherein the genotyping assay comprises an allele-specific oligonucleotide (ASO) probe which specifically hybridizes to a G-allele at position 27 of SEQ ID NO: 1, or a C-allele in the complementary nucleic acid sequence of SEQ ID NO: 1
   49. The assay of paragraph 48, wherein the allele-specific oligonucleotide (ASO) probe is a nucleic acid probe and comprises a detectable signal or a means to generate a detectable signal.
   50. The assay of any of paragraphs 31 to 47, wherein the genotyping assay comprises at least one probe flanking position 27 of SEQ ID NO: 1.
   51. The assay of any of paragraphs 31 to 50, wherein the genotyping assay comprises at least one allele-specific oligonucleotide (ASO) primer that specifically hybridizes to the G-allele at position 27 of SEQ ID NO: 1.
   52. The assay of any of paragraphs 31 to 50, wherein the treatment regimen for the subject where the subject has at least one G-allele at the rs10757269 loci is selected from any combination of treatments in the group consisting of: an exercise program; control of blood pressure, decreased sugar intake, and/or decreased lipid levels, cessation of smoking, and administration of drug therapies including the administration of aspirin (with or without dipyridamole), clopidogrel, cilostazol, and/or pentoxifylline.
   53. The assay of any of paragraphs 31 to 50, wherein the treatment regimen for the subject where the subject has at least one G-allele at the rs10757269 loci is selected from any suitable treatment for peripheral arterial disease (PAD).
   54. An assay comprising:
a. performing the assay according to paragraphs 1-30; and
b. performing the assay according to paragraphs 31-53.
   55. A computer system for determining if a subject is at risk of having a major adverse event, the system comprising:

a measuring module configured to detect the levels of at least three biomarkers selected from beta-2 microglobulin, C-reactive protein (CRP) and cystatin C in a biological subject obtained from a subject; a storage module configured to store output data from the measuring module;

a comparison module adapted to compare the data stored on the storage module with a reference threshold levels for beta-2 microglobulin, C-reactive protein (CRP) and cystatin, and to provide a retrieved content, and

a display module for displaying whether there the levels of beta-2 microglobulin, C-reactive protein (CRP) and cystatin C are at or above the reference threshold level, wherein the levels of beta-2 microglobulin, C-reactive protein (CRP) and cystatin C above the reference threshold level for each of beta-2 microglobulin, C-reactive protein (CRP) and cystatin C are above the reference threshold level indicate the subject is at risk of having a major adverse event, and/or displaying levels of beta-2 microglobulin, C-reactive protein (CRP) and cystatin C measured present in the biological sample.

56. The system of 55, wherein if the comparison module determines that the levels of beta-2 microglobulin, C-reactive protein (CRP) and cystatin C in the biological sample obtained from the subject are at or above the reference threshold level, the display module displays a positive signal indicating that the subject is likely to be at risk of having a major adverse event, as compared to a subject who has levels of beta-2 microglobulin, C-reactive protein (CRP) and cystatin C below the reference threshold levels for beta-2 microglobulin, C-reactive protein (CRP) and cystatin C.
57. The system of any of paragraphs 55 to 56, wherein if the comparison module determines the levels of beta-2 microglobulin, C-reactive protein (CRP) and cystatin C in the biological sample obtained from the subject are below the reference threshold levels for beta-2 microglobulin, C-reactive protein (CRP) and cystatin C, the display module displays a negative signal indicating that the subject is not likely to be at risk of having a major adverse event, as compared to a subject who has levels of beta-2 microglobulin, C-reactive protein (CRP) and cystatin C at or above the reference threshold levels for beta-2 microglobulin, C-reactive protein (CRP) and cystatin C.
58. The system of any of paragraphs 55 to 57, further comprising creating a report based on the levels of beta-2 microglobulin, C-reactive protein (CRP) and cystatin C in the biological sample obtained from the subject as compared to the reference threshold levels for beta-2 microglobulin, C-reactive protein (CRP) and cystatin C.
59. A method of identifying a subject at risk of a major adverse event, the method comprising detecting in a biological sample taken from the subject presenting a symptom of an acute cardiac event, or BMI of 25-30 or greater than 30, for the level of at least three biomarkers selected from beta-2-microglobulin, c-reactive protein (CRP) and cystatin C, wherein combination of the levels of beta-2-microglobulin, c-reactive protein (CRP) and cystatin C equal to, or above a threshold reference level for each of beta-2-microglobulin, c-reactive protein (CRP) and cystatin C indicates that the subject is at risk of a major adverse event.
60. A method of identifying a subject suitable for treatment to prevent the occurrence of a major adverse event, the method comprising detecting in a biological sample taken from the subject presenting a symptom of an acute cardiac event, or BMI of 25-30 or greater than 30, for the level of at least three biomarkers selected from beta-2-microglobulin, c-reactive protein (CRP) and cystatin C, wherein the combination of the levels of beta-2-microglobulin, c-reactive protein (CRP) and cystatin C above threshold reference levels for each beta-2-microglobulin, c-reactive protein (CRP) and cystatin C indicates that the subject should undergo treatment to reduce the incidence of a major adverse event.
61. The method of any of paragraphs 59 or 60, wherein the levels of beta-2-microglobulin, c-reactive protein (CRP) and cystatin C are measured in a biological sample obtained from a subject who has fasted.
62. The method of any of paragraphs 59 to 61, wherein the biological sample is a blood-based biological sample, or urine sample.
63. The method of any of paragraphs 59 to 62, wherein the levels of levels of beta-2-microglobulin, c-reactive protein (CRP) and cystatin C are measured using an antibody, antibody fragment or protein-binding molecule or other protein-binding probe.
64. The method of any of paragraphs 59 or 63, wherein the antibody, antibody fragment or protein-binding molecule or other protein-binding probe is bound to a solid support.
65. The method of any of paragraphs 59 or 64, wherein the levels of beta-2-microglobulin, c-reactive protein (CRP) and cystatin C are measured using an immunoassay.
66. The method of paragraph 65, wherein the immunoassay is an ELISA.
67. The method of any of paragraphs 59 or 66, wherein the subject is a Caucasian subject.
68. The method of any of paragraphs 59 or 66, wherein the subject is a African-American, Hispanic, Asian-American or Asian subject.
69. The method of any of paragraphs 59 or 66, wherein the subject is of Asian-Indian, Pakistani, Middle Eastern or Pacific Islander ethnicity.
70. The method of any of paragraphs 59 or 69, wherein a treatment to prevent the occurrence a major adverse event is selected from the group of: an exercise program; control of blood pressure, reduced sugar intake, cessation of smoking and drug therapies selected from the group of aspirin (with or without dipyridamole), clopidogrel, cilostazol, and/or pentoxifylline.
71. A method comprising:
(a) assaying a biological sample from the subject to determine the levels of beta-2-microglobulin, c-reactive protein (CRP) and cystatin C;
(b) determining a level of beta-2-microglobulin, c-reactive protein (CRP) and cystatin C is equal to, or above a reference threshold level for each biomarker; and
(c) diagnosing the subject as in need of treatment or therapy to prevent the occurrence of a major adverse event.
   72. A method for treating a human subject with a risk of a major adverse event, comprising administering a treatment or therapy to prevent the occurrence of a major adverse event to a human subject who is determined to have a level of beta-2-microglobulin, c-reactive protein (CRP) and cystatin C equal to, or above a reference threshold level for each biomarker.
   73. The method of paragraphs 71 and 72, wherein the treatment or therapy to prevent the occurrence a major adverse event is selected from the group of: an exercise program; control of blood pressure, reduced sugar intake, cessation of smoking and drug therapies selected from the group of aspirin (with or without dipyridamole), clopidogrel, cilostazol, and/or pentoxifylline.
   74. The method of any of paragraphs 71 to 73, wherein the major adverse event is stroke, heart attack or death.
   75. The method of any of paragraphs 71 to 74, wherein the major adverse event is a major adverse cardiovascular or cerebrovascular event (MACCE).
   76. The method of paragraph 75, wherein the MACCE is selected from the group consisting of: recurrence of an initial cardiac event, angina, decompensation of heart failure, admission for cardiovascular disease (CVD), mortality due to CVD, and transplant.
   77. The method of any of paragraphs 71 to 76, wherein threshold reference level for beta-2-microglobulin is 1.88 mg/l.
   78. The method of any of paragraphs 71 to 77, wherein threshold reference level for CRP is 1.60 mg/l.
   79. The method of any of paragraphs 71 to 78, wherein threshold reference level for cystatin C is 0.72 mg/l.
   80. An assay to select a subject at risk of having a major adverse event, the assay comprising:

contacting a biological sample obtained from the subject with at least one probe to detect the levels of at least three biomarkers selected from beta-2 microglobulin, C-reactive protein (CRP) and cystatin C; measuring the levels of at least three biomarkers selected from beta-2 microglobulin, C-reactive protein (CRP) and cystatin C;

wherein the level of beta-2 microglobulin, C-reactive protein (CRP) and cystatin C above a threshold reference level for each of beta-2 microglobulin, C-reactive protein (CRP) and cystatin C, thereby selecting a subject at risk of having a major adverse event.

81. An assay comprising:
a. measuring the levels of antibodies that are reactive to at least three biomarkers selected from beta-2 microglobulin, C-reactive protein (CRP), and cystatin C in a biological sample obtained from a subject who has a body mass index (BMI) of 25 or greater for determining the likelihood of the subject having a major adverse event; and
b. selecting a subject having an increased level of the antibodies of the least three biomarkers in the biological sample relative to a reference antibody level for each of beta-2 microglobulin, C-reactive protein (CRP) and cystatin C, as being at risk of having a major adverse event.

### EXAMPLES

The examples presented herein relate to the methods, kits, machines and computer systems and media to determine the levels of biomarkers beta 2 microglobulin, CRP and cystatin C in the plasma or serum to identify a subject at risk of a major adverse event, such as but not limited to a heart attack, stroke or death. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Materials and Methods:

Study Population.

The Genetic Determinants of Peripheral Arterial Disease (GenePAD) study consists of individuals who underwent an elective, non-emergent coronary angiogram for angina, shortness of breath or an abnormal stress test at Stanford University or Mount Sinai Medical Centers between Jan. 1, 2004 and Mar. 1, 2008.^{21,22} As previously described²³, a subgroup of 470 individuals was selected to characterize the role of biomarkers in PAD. The GenePAD study was approved by the Stanford University and Mount Sinai School of Medicine Committees for the Protection of Human Subjects.

Inclusion Criteria.

Individuals were eligible for inclusion in the study sample if complete data was available on rs10757269, the biomarkers beta-2-microglobulin, cystatin C, C-reactive protein and plasma glucose in addition to age, sex, race, smoking history, body mass index (BMI), systolic blood pressure (SBP), use of lipid-lowering and anti-hypertensive medications, use of insulin or oral hypoglycemic agents, total cholesterol, high-density lipoprotein (HDL) cholesterol, ankle-brachial index (ABI) and history of CAD, CVD, and congestive heart failure (CHF). Additionally, the inventors included Caucasian, African-American and Asian-American individuals as other polymorphisms (different to the rs10757269) at the 9p21 locus has previously been shown to be potentially predictive of cardiovascular events in these racial-ethnic groups (Ding et al. Circ Cardiovasc Genet. 2009; 2: 338-46; Heckman et al., Eur J Neurol. 2013; 20: 300-8; Shiffman et al., BMC Cardiovasc Disord. 2011; 11: 10; Murabito et al., Circ Cardiovasc Genet. 2012; 5: 100-12). Using these criteria, 393 subjects were identified from the original cohort of 470 individuals, and were included for study.

Covariates.

Prior to the coronary angiogram, posterior tibial, dorsalis pedis, and brachial artery systolic pressures were measured using a 5 MHz Doppler ultrasound. The ABI for each patient was calculated by dividing the higher ankle pressure of each leg over the higher of the left or right brachial pressures. Each patient was then classified as having peripheral arterial disease by an ABI of <0.9 in either leg or not having PAD with an ABI≧0.9 in both legs.

Detailed information on all included covariates was obtained by a trained nurse or clinical research assistant at enrollment. Age, sex, race, smoking history and history of CVD, CHF and CAD were acquired by self-report and BMI and SBP were measured. The use of lipid-lowering and anti-hypertensive medications was evaluated by direct medication inventory. Diabetes status was classified as self-reported use of insulin or oral hypoglycemic agents. Total and HDL cholesterol levels were measured at the time of coronary angiography. The biomarkers were measured with standard nephelometry using BNII-Nephelometry system (Dade Behring Inc.) using fasting blood samples collected while the patient was being prepped for scheduled coronary angiography. Patients completed the Walking Impairment Questionnaire (WIQ) at enrollment with a trained nurse or clinical research assistant as previously described in the GenePAD study²⁹. The WIQ consists of three categories assessing subjective walking distance, stair-climbing and walking speed ability and has been previously validated as a measure of objective walking distance.

Statistical Methods.

The biomarkers beta-2-microglobulin, cystatin C, C-reactive protein and plasma glucose were log-transformed to achieve a normal distribution. The association of rs10757269 with PAD was tested using a multivariable logistic regression analysis and for association with ABI and the WIQ category scores using a multivariable linear regression analysis. The fully adjusted model included beta-2-microglobulin, cystatin C, C-reactive protein, plasma glucose, age, sex, race, smoking history, BMI, hypertension stage, use of lipid-lowering and anti-hypertensive medications, diabetes status, total cholesterol and HDL cholesterol. All covariates were continuous except race (categorical), smoking, use or nonuse of lipid-lowering and anti-hypertensive medications and diabetes status (dichotomous).

The integrated discrimination improvement (IDI) and the net reclassification improvement (NRI) were evaluated to determine whether the addition of rs10757269 to a baseline model significantly improved risk discrimination and reclassification respectively (Pencina et al., Stat Med. 2008; 27: 157-72). In this analysis, the inventors used a baseline model previously validated for PAD that included the risk factors age, sex, race, smoking history, BMI, hypertension stage, diabetes status and history of CVD, CHF, and CAD (Duval et al., Vasc Med. 2012; 17: 342-51). The IDI compares two models according to the average difference in predicted risk between those who have the outcome and those who do not. If the new model assigns a higher risk to those who have PAD and a lower risk to those who do not, as compared to the baseline model, the IDI will be greater than zero. Therefore, the IDI can be interpreted as the average net improvement in the predicted risk of PAD in the model with rs10757269 compared to the baseline model.

The category-free NRI was used in this study, as a priori risk categories do not exist. This NRI quantifies the degree of correct upward or downward absolute risk reclassification with the addition of rs10757269 to the baseline model. Furthermore, the NRI was calculated separately among individuals with and without PAD.

Tests were considered significant if the two-sided P-value was <0.05. All analyses were performed using Stata version 12.0 (StataCorp, College Station, Tex.). Study data were collected and managed using REDCap electronic data capture tools hosted at Stanford University (Harris et al., J Biomed Inform. 2009; 42: 377-81).

### Example 1

The Genetic Determinants of Peripheral Arterial Disease (GenePAD) study consists of individuals who underwent an elective, non-emergent coronary angiogram for angina, shortness of breath or an abnormal stress test at Stanford University or Mount Sinai Medical Centers between Jan. 1, 2004 and Mar. 1, 2008^{3,4}. As previously detailed⁵, a sub-cohort of individuals was selected from the total cohort (n=1755) to characterize the role of biomarkers in cardiovascular disease. There were 470 patients with data on all biomarkers and relevant covariates included in this study. All individuals provided written informed consent. The GenePAD study was approved by the Stanford University and Mount Sinai School of Medicine Committees for the Protection of Human Subjects.

The biomarkers assessed were beta-2-microglobulin, cystatin C and C-reactive protein. Fasting blood samples were collected while the patient was being prepped for scheduled coronary angiography. The biomarkers were measured with standard nephelometry using BNII-Nephelometry system (Dade Behring Inc.). The intra-assay and inter-assay coefficients of variation were <4.1% and <3.3% for beta-2-microglobulin, <4.4% and <5.7% for cystatin C, and <2.83% and <5.1% for C-reactive protein respectively.

The outcomes of interest in this analysis were death from any cause and from cardiovascular causes. Cardiovascular deaths were attributed to myocardial infarction, cardiac arrest, stroke, heart failure or aneurysm rupture. Ascertainment of mortality was achieved through phone or postal communication, medical record review and the Social Security Death Index. New mortalities were identified through Mar. 31, 2012.

At enrollment, participants provided information on all included covariates through a trained nurse or research assistant. Diabetes status was classified as use of insulin or oral hypoglycemic agents as ascertained by direct medication inventory. Total cholesterol and high-density lipoprotein (HDL) cholesterol were measured by standard assays using AU5400 Chemistry Immuno-Analyzer (Olympus Inc.). The glomerular filtration rate (GFR) was estimated using the Modification of Diet in Renal Disease method⁶. An experienced cardiologist who was blinded to participant details evaluated coronary angiograms. Hemodynamically significant coronary artery disease (CAD) was defined as >60% stenosis^{7,8}.

Cumulative mortality for all-cause and cardiovascular mortality was calculated for each biomarker using the Kaplan-Meier method with the median level for each biomarker as the designated cut-off value between groups. Additionally, participants in the upper 50% for all three biomarkers were compared to those in the lower 50% for all three biomarkers.

Continuous variables with a right-skew (beta-2-microglobulin, cystatin C and C-reactive protein) were log-transformed to achieve a normal distribution. The association of biomarkers with death from all causes and death from cardiovascular causes was investigated using Cox proportional-hazards regression. Hazard ratios were expressed per 1-standard deviation change of the log biomarker level. Standard deviations were 6.4, 0.98 and 7.0 mg/L for beta-2-microglobulin, cystatin C and C-reactive protein respectively. Subgroup analysis was carried out for all-cause mortality according to CAD status. Due to limited numbers of cardiovascular mortalities (n=19) the inventors elected not to undertake subgroup analysis on this outcome.

For all survival analyses the follow-up time was defined as the period between the enrollment interview and the last confirmed follow-up or date of death. If participants had a confirmed mortality of unknown cause they were excluded from the cardiovascular mortality analysis (n=48). Survival analyses were adjusted for age, sex, race, systolic blood pressure (SBP), body mass index (BMI), total cholesterol, HDL cholesterol, smoking history, use of lipid-lowering and anti-hypertensive medications, use of insulin or oral hypoglycemic agents, and GFR. All variables were continuous except race (categorical), diabetes status, smoking, and use or nonuse of lipid-lowering and anti-hypertensive medications (dichotomous). Proportional-hazards assumptions were evaluated by Schoenfeld's residuals tests. Calibration was assessed on all models using the Gronnesby-Borgan test to evaluate goodness-of-fit (P≧0.05) by comparing predicted mortalities with observed mortalities as described for survival analysis⁹.

The net reclassification improvement (NRI), C-index and integrated discrimination improvement (IDI) were evaluated to determine whether the biomarkers significantly improved risk reclassification and discrimination for all-cause and cardiovascular mortality when added to a baseline model. In this diverse population at high-risk for cardiovascular events, the inventors used a baseline model consisting of risk factors for cardiovascular disease and death including age, sex, race, smoking history, BMI, SBP, use of lipid-lowering or anti-hypertensive medications, diabetes, total cholesterol, HDL cholesterol and GFR¹⁰⁻¹³. Additionally, secondary analyses were conducted using risk variables from the European SCORE risk model to evaluate model improvement against an established risk score¹². This model was established for cardiovascular mortality, and includes age, sex, smoking history, SBP and total cholesterol.

The NRI was used to evaluate the proportion of correct risk reclassification when adding biomarkers to the baseline model¹⁴. The inventors utilized the category-free NRI as it has been suggested to be the most objective and reproducible measure of improvement in risk prediction especially when established a priori risk categories do not exist¹⁵. Furthermore, the inventors calculated the NRI separately in participants with and without an event during follow-up.

The C-index was used to estimate improvements in model discrimination with the addition of the biomarkers. In survival analysis, the C-index interpretation is equivalent to the area under the ROC curve or c-statistic, while allowing for censored data with a 1% increase indicating that the correct order of failure (e.g. mortality) would be correctly predicted in an additional 1 in every 100 pairs of randomly selected individuals compared to the baseline model^{16,17}.

Model performance was further evaluated with the addition of the biomarkers using the IDI. The IDI compares two models according to the average difference in predicted risk between those who have the event and those who do not¹⁴. If the new model assigns a higher risk to those who will have a mortality and a lower risk to those who will not, as compared to the baseline model, the IDI will be >0. Therefore, the IDI can be interpreted as the average net improvement in the predicted risk of the outcome in the new model compared to the baseline model.

Tests were considered significant if the two-sided P-value was <0.05. All analyses were performed using Stata version 12.0 (StataCorp, College Station, Tex.). Study data were collected and managed using REDCap electronic data capture tools hosted at Stanford University¹⁸.

### Example 2

Enrollment characteristics of the 470 individuals constituting the study sample are presented in Table 1. During a median follow-up period of 5.6 years there were 78 mortalities (17%) of which 19 were known to be from cardiovascular causes.

TABLE 1
Baseline study population characteristics (n = 470).

| Characteristic | Value | |
|---|---|---|
| Age, mean (years) | 67 ± 10 | |
| Female | 226 | (48%) |
| Caucasian | 253 | (54%) |
| Black | 77 | (16%) |
| Hispanic | 58 | (12%) |
| Asian | 33 | (7%) |
| Other* | 49 | (10%) |
| Systolic blood pressure, mean (mm Hg) | 141 ± 22 | |
| Body mass index, mean (kg/m²) | 29 ± 6 | |
| Lipids, mean (mg/dl) | | |
| Total cholesterol | 145 ± 38 | |
| High-density lipoprotein cholesterol | 42 ± 13 | |
| Ever smoker | 267 | (57%) |
| Use of cholesterol lowering medication | 301 | (64%) |
| Use of antihypertensive medication | 391 | (83%) |
| Use of insulin or oral hypoglycemics | 146 | (31%) |
| Glomerular filtration rate, mean (mL/min/1.73 m²) | 79 ± 37 | |
| Biomarker levels, median (mg/L) (IQR) | | |
| Beta-2-microglobulin | 1.88 | (1.50-2.57) |
| Cystatin C | 0.72 | (0.61-0.93) |
| C-reactive protein | 1.60 | (0.60-4.30) |
| Coronary artery disease (CAD)† | 219 | (47%) |

*Includes Asian-Indian, Pakistani, Middle Eastern and Pacific Islander.
†Defined as >60% stenosis on coronary angiography. All mean values are presented ± the standard deviation
IQR, interquartile range; No., number.

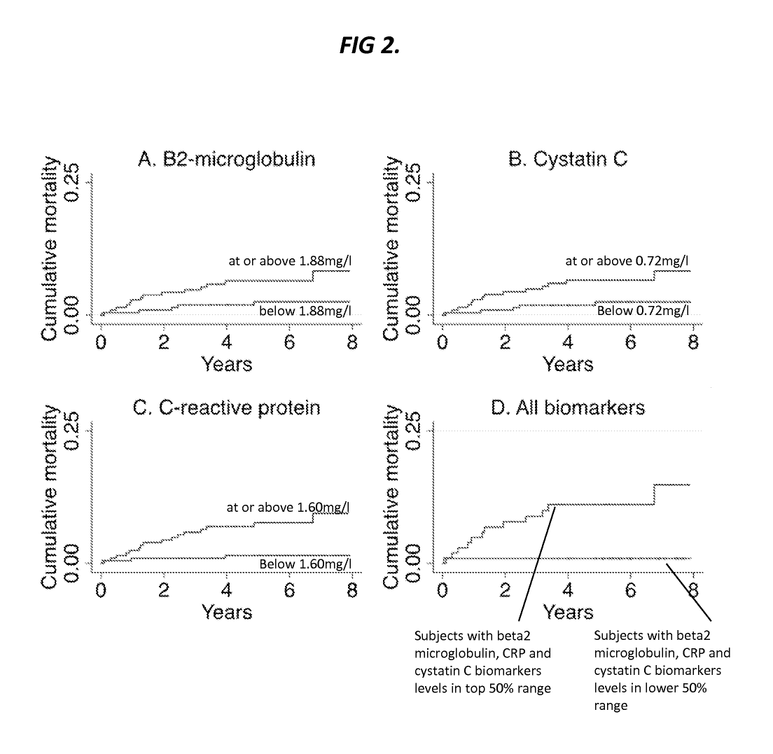

The inventors discovered an increased cumulative all-cause mortality (FIG. 1) and cardiovascular mortality (FIG. 2) among individuals with levels of beta-2-microglobulin, cystatin C or C-reactive protein that were greater than the study median. This relationship was most pronounced when comparing participants with measurements above the median for all biomarkers as compared to below the median for all biomarkers.

The adjusted hazard ratios for the association of all biomarkers with mortality are shown in Table 2.

TABLE 2
Adjusted hazard ratios per standard deviation
increase in log biomarker level.

| | | | 95% CI | | |
|---|---|---|---|---|---|
| | | HR | Lower | Upper | P-value |
| All-cause mortality | | | | | |
| Beta-2-microglobulin | | | | | |
| | Overall | 1.80 | 1.38 | 2.34 | <0.001 |
| | CAD only | 1.75 | 1.20 | 2.56 | 0.004 |
| | Non CAD | 1.96 | 1.24 | 3.10 | 0.004 |
| Cystatin C | | | | | |
| | Overall | 1.74 | 1.31 | 2.29 | <0.001 |
| | CAD only | 1.79 | 1.20 | 2.65 | 0.004 |
| | Non CAD | 1.61 | 0.98 | 2.63 | 0.060 |
| C-reactive protein | | | | | |
| | Overall | 1.70 | 1.37 | 2.10 | <0.001 |
| | CAD only | 1.67 | 1.28 | 2.17 | <0.001 |
| | Non CAD | 1.66 | 1.04 | 2.66 | 0.035 |
| Cardiovascular | | | | | |
| mortality | | | | | |
| Beta-2-microglobulin | Overall | 2.25 | 1.34 | 3.77 | 0.002 |
| Cystatin C | Overall | 2.35 | 1.40 | 3.93 | 0.001 |
| C-reactive protein | Overall | 1.96 | 1.24 | 3.09 | 0.004 |

Data were adjusted for age, sex, race, smoking history, body mass index, systolic blood pressure, use of lipid-lowering or anti-hypertensive medications, diabetes, total cholesterol, high-density lipoprotein cholesterol and glomerular filtration rate. CAD, coronary artery disease; CI, confidence interval; HR, hazard ratio; SD, standard deviation.

Higher levels of the biomarkers beta-2-microglobulin, cystatin C and C-reactive protein were significantly associated with increased all-cause and cardiovascular mortality during follow-up. The observed associations did not significantly differ according to gender or race (P>0.05). The inventors therefore also conducted analyses using fasting glucose as an alternative measure of diabetes status, which yielded statistically similar results (data not shown). Schoenfeld's residuals tests demonstrated that the proportional hazards assumption was met for all models. Regression coefficients for the all-cause mortality analysis can be found in Table 3.

TABLE 3
Regression coefficients for single biomarker
all-cause mortality Cox regression models.

| | Beta-2- | | C-reactive |
|---|---|---|---|
| | microglobulin | Cystatin C | protein |
| Biomarker | 0.588 | 0.552 | 0.528 |
| Age | 0.053 | 0.048 | 0.033 |
| Sex | −0.293 | −0.295 | −0.447 |
| Race | −0.088 | −0.090 | −0.077 |
| Systolic blood pressure | 0.008 | 0.008 | 0.008 |
| Body mass index | −0.019 | −0.024 | 0.000 |
| Total cholesterol | −0.004 | −0.005 | −0.005 |
| High-density lipoprotein | 0.015 | 0.016 | 0.017 |
| cholesterol | | | |
| Ever smoker | 0.244 | 0.265 | 0.217 |
| Use of cholesterol lowering | −0.273 | −0.317 | −0.385 |
| medication | | | |
| Use of antihypertensive | 0.529 | 0.529 | 0.370 |
| medication | | | |
| Use of insulin or oral | 0.568 | 0.605 | 0.573 |
| hypoglycemics | | | |
| Glomerular filtration rate | 0.006 | 0.005 | −0.011 |

In subgroup analysis, beta-2-microglobulin, cystatin C and C-reactive protein were predictive of all-cause mortality among individuals with CAD diagnosed at enrollment. Beta-2-microglobulin and C-reactive protein continued to significantly predict mortality risk among individuals without CAD while cystatin C demonstrated a borderline significance in this subgroup.

Assessment of calibration using the Gronnesby-Borgan statistic demonstrated good fit for all models with and without biomarkers (P≧0.05).

The category-free NRI showed significant improvement in the net proportion of risk reclassification for all models with the addition of beta-2-microglobulin, cystatin C and C-reactive protein, individually and combined, compared to the baseline risk factors model for both all-cause and cardiovascular mortality (Table 4).

TABLE 4
Category-free net reclassification improvement
over baseline risk factors.

| | Overall | | NRI | NRI Non- |
|---|---|---|---|---|
| Model | NRI | P-value | Mortalities | mortalities |
| All-cause mortality | | | | |
| Baseline risk factors (BRF)* | ref | 1.0 (ref) | ref | ref |
| BRF + Beta-2-microglobulin | 25.0% | 0.044 | 0.0% | 25.0% |
| BRF + Cystatin C | 27.0% | 0.029 | 0.0% | 27.0% |
| BRF + C-reactive protein | 45.0% | <0.001 | 23.1% | 21.9% |
| BRF + all biomarkers | 35.8% | 0.004 | 10.3% | 25.5% |
| Cardiovascular mortality | | | | |
| Baseline risk factors (BRF) | ref | 1.0 (ref) | ref | ref |
| BRF + Beta-2-microglobulin | 54.9% | 0.019 | 26.3% | 28.5% |
| BRF + Cystatin C | 72.9% | 0.002 | 47.4% | 25.6% |
| BRF + C-reactive protein | 66.0% | 0.005 | 47.4% | 18.6% |
| BRF + all biomarkers | 61.9% | 0.008 | 36.8% | 25.1% |

*Age, gender, race, smoking history, body mass index, systolic blood pressure, use of lipid-lowering medication, use of anti-hypertensive medication, diabetes status, total cholesterol, high-density lipoprotein cholesterol and glomerular filtration rate, NRI, net reclassification improvement; ref, reference.

Results for the C-index and IDI analyses are presented in Table 5. The baseline cardiovascular risk factors model had a C-index of 0.720 (95% CI, 0.660-0.780) and 0.755 (95% CI, 0.650-0.860) for all-cause and cardiovascular mortality, respectively. As compared to the baseline model, beta-2-microglobulin and C-reactive protein demonstrated significantly improved model risk discrimination for all-cause mortality. None of the three biomarkers significantly improved cardiovascular mortality risk discrimination individually using the C-index. However, the addition of all three biomarkers showed the largest magnitude of increased C-index for all-cause and cardiovascular mortality respectively.

TABLE 5
C-index and integrated discrimination improvement over baseline risk factors.

| | C-Index | | | IDI | |
|---|---|---|---|---|---|
| Model | C† | ΔC (95% CI) | P-value | IDI (95% CI) | P-value |
| All-cause mortality | | | | | |
| Baseline risk factors (BRF)* | 0.720 | ref | ref (1.0) | ref | ref (1.0) |
| BRF + Beta-2-microglobulin | 0.756 | 0.036 (0.007-0.065) | 0.016 | 1.9% (0.3-3.5%) | 0.017 |
| BRF + Cystatin C | 0.745 | 0.025 (−0.001-0.050) | 0.061 | 1.6% (0.3-2.8%) | 0.018 |
| BRF + C-reactive protein | 0.756 | 0.036 (0.001-0.072) | 0.046 | 4.0% (1.9-6.1%) | <0.001 |
| BRF + all biomarkers | 0.777 | 0.057 (0.016-0.097) | 0.006 | 5.1% (2.6-7.6%) | <0.001 |
| Cardiovascular mortality | | | | | |
| Baseline risk factors (BRF) | 0.755 | ref | ref (1.0) | ref | ref (1.0) |
| BRF + Beta-2-microglobulin | 0.813 | 0.058 (−0.003-0.118) | 0.062 | 2.1% (−0.2-4.4%) | 0.077 |
| BRF + Cystatin C | 0.814 | 0.059 (−0.001-0.118) | 0.055 | 2.9% (−0.1-5.9%) | 0.056 |
| BRF + C-reactive protein | 0.796 | 0.041 (−0.017-0.099) | 0.166 | 1.8% (0.1-3.5%) | 0.042 |
| BRF + all biomarkers | 0.826 | 0.071 (0.010-0.133) | 0.023 | 3.8% (−0.1-7.8%) | 0.058 |

*Age, gender, race, smoking history, body mass index, systolic blood pressure, use of lipid-lowering medication, use of anti-hypertensive medication, diabetes status, total cholesterol, high-density lipoprotein cholesterol and glomerular filtration rate
†The C-index for all models was significantly greater than the null hypothesis of 0.5 at P < 0.001.
C, C-index; CI, confidence interval; ΔC, change in C-index from the reference model; IDI, integrated discrimination improvement; ref, reference

The IDI demonstrated a significant average net improvement in the predicted risk of all-cause mortality with the individual addition of beta-2-microglobulin, cystatin C and C-reactive protein (Table 5). Only C-reactive protein significantly improved the IDI for cardiovascular mortality with cystatin C showing a borderline significance. The models including all three biomarkers demonstrated the largest IDI for all-cause mortality and for cardiovascular mortality.

The results of the addition of biomarkers to the model consisting of SCORE risk variables are presented in Tables 6 and 7. These analyses demonstrated statistically significant improvement for all measures of risk discrimination and reclassification for all-cause mortality using the NRI, C-index and IDI. For cardiovascular mortality, all biomarkers significantly improved risk reclassification per the NRI, individually and combined, over the SCORE risk variables model. Estimated IDI values were consistent with improved discrimination but did not reach statistical significance. However, compared to the baseline model of SCORE variables, all biomarkers significantly improved the C-index with the three biomarker model resulting in a C-index of 0.806 (P=0.007).

Additionally, the inventors examined the NRI, C-index and IDI according to CAD status for all causes of mortality compared to the baseline risk factors model (Tables 6 and 7). The addition of all three biomarkers significantly improved risk reclassification and discrimination among individuals both with and without CAD at enrollment (P<0.05).

TABLE 6
Category-free net reclassification
improvement over SCORE risk factors

| | Overall | | NRI | NRI Non- |
|---|---|---|---|---|
| Model | NRI | P-value | Mortalities | mortalities |
| All-cause mortality | | | | |
| SCORE risk factors (SRF)* | ref | 1.0 (ref) | ref | ref |
| SRF + B2-microglobulin | 54.1% | <0.001 | −2.6% | 56.6% |
| SRF + Cystatin C | 54.1% | <0.001 | 0.0% | 54.1% |
| SRF + C-reactive protein | 56.3% | <0.001 | 28.2% | 28.1% |
| SRF + all biomarkers | 56.2% | <0.001 | 15.4% | 40.8% |
| Cardiovascular mortality | | | | |
| SCORE risk factors (SRF) | ref | 1.0 (ref) | ref | ref |
| SRF + B2-microglobulin | 71.1% | 0.002 | 15.8% | 55.3% |
| SRF + Cystatin C | 66.2% | 0.005 | 15.8% | 50.4% |
| SRF + C-reactive protein | 82.5% | 0.001 | 57.9% | 24.6% |
| SRF + all biomarkers | 74.2% | 0.002 | 26.3% | 47.9% |

*Age, sex, smoking history, systolic blood pressure and total cholesterol
NRI, net reclassification improvement; ref, reference.

TABLE 7
C-index and integrated discrimination improvement over SCORE risk factors

| | C-Index | | | IDI | |
|---|---|---|---|---|---|
| Model | C† | ΔC (95% CI) | P-value | IDI (95% CI) | P-value |
| All-cause mortality | | | | | |
| SCORE risk factors (SRF)* | 0.656 | ref | ref (1.0) | ref | ref (1.0) |
| SRF + Beta-2-microglobulin | 0.731 | 0.075 (0.026-0.124) | 0.003 | 3.0% (0.8-5.1%) | 0.006 |
| SRF + Cystatin C | 0.719 | 0.062 (0.015-0.109) | 0.01 | 2.6% (0.8-4.5%) | 0.003 |
| SRF + C-reactive protein | 0.723 | 0.067 (0.024-0.110) | 0.002 | 4.3% (2.1-6.6%) | <0.001 |
| SRF + all biomarkers | 0.757 | 0.101 (0.047-0.154) | <0.001 | 6.1% (3.0-9.3%) | <0.001 |
| Cardiovascular mortality | | | | | |
| SCORE risk factors (SRF) | 0.685 | ref | ref (1.0) | ref | ref (1.0) |
| SRF + Beta-2-microglobulin | 0.769 | 0.084 (0.000-0.168) | 0.050 | 1.2% (−0.8-3.2%) | 0.255 |
| SRF + Cystatin C | 0.770 | 0.085 (0.004-0.166) | 0.039 | 1.4% (−0.6-3.4%) | 0.164 |
| SRF + C-reactive protein | 0.771 | 0.086 (0.009-0.164) | 0.030 | 1.0% (−0.1-2.0%) | 0.077 |
| SRF + all biomarkers | 0.805 | 0.119 (0.032-0.206) | 0.007 | 2.1% (−0.7-4.8%) | 0.138 |

*Age, sex, smoking history, systolic blood pressure and total cholesterol
†The C-index for all models was significantly greater than the null hypothesis of 0.5 at P < 0.001
C, C-index; CI, confidence interval; ΔC, change in C-index from the reference model; IDI, integrated
discrimination improvement; ref, reference.

TABLE 8
Category-free net reclassification improvement for all-
cause mortality by coronary artery disease status.

Overall

| Model | Estimate | P-value |
|---|---|---|
| Coronary artery disease at enrollment | | |
| Baseline risk factors (BRF)* | ref | 1.0 (ref) |
| BRF + Beta-2-microglobulin | 12.8% | 0.414 |
| BRF + Cystatin C | 34.6% | 0.027 |
| BRF + C-reactive protein | 60.2% | <0.001 |
| BRF + all biomarkers | 60.1% | <0.001 |
| No coronary artery disease at enrollment | | |
| Baseline risk factors (BRF)* | ref | 1.0 (ref) |
| BRF + Beta-2-microglobulin | 30.5% | 0.148 |
| BRF + Cystatin C | 33.2% | 0.115 |
| BRF + C-reactive protein | 6.6% | 0.753 |
| BRF + all biomarkers | 47.4% | 0.025 |

*Age, gender, race, smoking history, body mass index, systolic blood pressure, use of lipid-lowering medication, use of anti-hypertensive medication, diabetes status, total cholesterol, high-density lipoprotein cholesterol and glomerular filtration rate.
NRI, net reclassification improvement

TABLE 9
C-index and integrated discrimination improvement for all-cause mortality

| | C-Index | | | IDI | |
|---|---|---|---|---|---|
| Model | C† | ΔC (95% CI) | P-value | Estimate | P-value |
| Coronary artery disease | | | | | |
| at enrollment | | | | | |
| Baseline risk factors (BRF)* | 0.719 | ref (0.0) | ref (1.0) | ref | ref (1.0) |
| BRF + Beta-2-microglobulin | 0.741 | 0.022 (−0.016-0.061) | 0.257 | 2.10% | 0.075 |
| BRF + Cystatin C | 0.738 | 0.019 (−0.021-0.060) | 0.348 | 2.40% | 0.033 |
| BRF + C-reactive protein | 0.753 | 0.034 (−0.015-0.083) | 0.168 | 5.20% | <0.001 |
| BRF + all biomarkers | 0.763 | 0.044 (−0.011-0.099) | 0.118 | 6.60% | <0.001 |
| No coronary artery | | | | | |
| disease at enrollment | | | | | |
| Baseline risk factors (BRF)* | 0.711 | ref (0.0) | ref (1.0) | ref | ref (1.0) |
| BRF + Beta-2-microglobulin | 0.747 | 0.036 (−0.008-0.079) | 0.109 | 2.80% | 0.065 |
| BRF + Cystatin C | 0.729 | 0.017 (−0.013-0.048) | 0.254 | 1.20% | 0.204 |
| BRF + C-reactive protein | 0.729 | 0.018 (−0.041-0.077) | 0.546 | 1.50% | 0.172 |
| BRF + all biomarkers | 0.753 | 0.042 (−0.031-0.115) | 0.259 | 6.90% | 0.005 |

*Age, gender, race, smoking history, body mass index, systolic blood pressure, use of lipid-lowering medication, use of anti-hypertensive medication, diabetes status, total cholesterol, high-density lipoprotein cholesterol and glomerular filtration rate.
†The C-index for all models was significantly greater than the null hypothesis of 0.5 at P < 0.001.
C, C-index; CI, confidence interval; ΔC, change in C-index from the reference model; IDI, integrated discrimination improvement; ref, reference

Additionally, the inventors determined that the biomarkers can predict MACCE (major adverse cardiovascular and cerebrovascular event), as well as individual events, such as stroke, heart failure, coronary bypass, as shown in Table 10. The p values in table 10 show significance (univariate and controlled for other risk factors, etc.).

TABLE 10
Data were adjusted for age, sex, race, smoking history, body mass index, systolic
blood pressure, use of lipid-lowering or anti-hypertensive medications, diabetes, total
cholesterol, high-density lipoprotein cholesterol and glomerular filtration rate.
Table 10:

| | | | | |
|---|---|---|---|---|
| Marker | OR | Lower CI | Upper CI | P-value |

First MACCE

| | | | | | |
|---|---|---|---|---|---|
| Beta-2-microglobulin | 1.24 | 1.07 | 1.44 | 0.004 | Age, gender adjusted |
| Cystatin C | 1.29 | 1.12 | 1.49 | 0.000 | |
| C-reactive protein | 1.27 | 1.07 | 1.52 | 0.007 | |
| Beta-2-microglobulin | 1.22 | 1.03 | 1.43 | 0.020 | Age, gender, LDL cholesterol, |
| Cystatin C | 1.27 | 1.08 | 1.49 | 0.003 | smoking, systolic blood pressure |
| C-reactive protein | 1.23 | 1.01 | 1.50 | 0.037 | adjusted |
| Beta-2-microglobulin | 1.24 | 0.97 | 1.59 | 0.082 | Age, gender, race, smoking |
| Cystatin C | 1.35 | 1.05 | 1.73 | 0.020 | history, body mass index, systolic |
| C-reactive protein | 1.20 | 0.98 | 1.47 | 0.073 | blood pressure, use of lipid- |
| | | | | | lowering medication, use of anti- |
| | | | | | hypertensive medication, diabetes |
| | | | | | status, total cholesterol, high- |
| | | | | | density lipoprotein cholesterol and |
| | | | | | glomerular filtration rate adjusted |

Stroke

| | | | | | |
|---|---|---|---|---|---|
| Beta-2-microglobulin | 1.59 | 1.00 | 2.53 | 0.051 | Age, gender adjusted |
| Cystatin C | 1.69 | 1.06 | 2.70 | 0.028 | |
| C-reactive protein | 1.31 | 0.70 | 2.44 | 0.399 | |
| Beta-2-microglobulin | 1.60 | 0.99 | 2.57 | 0.054 | Age, gender, LDL cholesterol, |
| Cystatin C | 1.71 | 1.06 | 2.77 | 0.029 | smoking, systolic blood pressure |
| C-reactive protein | 1.33 | 0.66 | 2.66 | 0.422 | adjusted |
| Beta-2-microglobulin | 2.24 | 0.88 | 5.68 | 0.089 | Age, gender, race, smoking |
| Cystatin C | 3.21 | 1.13 | 9.16 | 0.029 | history, body mass index, systolic |
| C-reactive protein | 1.24 | 0.59 | 2.64 | 0.571 | blood pressure, use of lipid- |
| | | | | | lowering medication, use of anti- |
| | | | | | hypertensive medication, diabetes |
| | | | | | status, total cholesterol, high- |
| | | | | | density lipoprotein cholesterol and |
| | | | | | glomerular filtration rate adjusted |

Heart Failure

| | | | | | |
|---|---|---|---|---|---|
| Beta-2-microglobulin | 1.42 | 0.97 | 2.07 | 0.073 | Age, gender adjusted |
| Cystatin C | 1.46 | 1.00 | 2.13 | 0.049 | |
| C-reactive protein | 1.67 | 1.00 | 2.78 | 0.050 | |
| Beta-2-microglobulin | 1.48 | 1.00 | 2.20 | 0.050 | Age, gender, LDL cholesterol, |
| Cystatin C | 1.53 | 1.03 | 2.27 | 0.033 | smoking, systolic blood pressure |
| C-reactive protein | 1.85 | 1.08 | 3.17 | 0.026 | adjusted |
| Beta-2-microglobulin | 1.69 | 0.90 | 3.19 | 0.105 | Age, gender, race, smoking |
| Cystatin C | 1.81 | 0.93 | 3.55 | 0.082 | history, body mass index, systolic |
| C-reactive protein | 1.78 | 1.01 | 3.15 | 0.047 | blood pressure, use of lipid- |
| | | | | | lowering medication, use of anti- |
| | | | | | hypertensive medication, diabetes |
| | | | | | status, total cholesterol, high- |
| | | | | | density lipoprotein cholesterol and |
| | | | | | glomerular filtration rate adjusted |

Coronary bypass

| | | | | | |
|---|---|---|---|---|---|
| Beta-2-microglobulin | 1.22 | 1.04 | 1.44 | 0.014 | Age, gender adjusted |
| Cystatin C | 1.29 | 1.10 | 1.50 | 0.002 | |
| C-reactive protein | 1.27 | 1.05 | 1.53 | 0.014 | |
| Beta-2-microglobulin | 1.19 | 0.99 | 1.43 | 0.070 | Age, gender, LDL cholesterol, |
| Cystatin C | 1.25 | 1.05 | 1.49 | 0.011 | smoking, systolic blood pressure |
| C-reactive protein | 1.21 | 0.98 | 1.49 | 0.076 | adjusted |
| Beta-2-microglobulin | 1.20 | 0.92 | 1.57 | 0.178 | Age, gender, race, smoking |
| Cystatin C | 1.33 | 1.02 | 1.74 | 0.037 | history, body mass index, systolic |
| C-reactive protein | 1.17 | 0.95 | 1.46 | 0.147 | blood pressure, use of lipid- |
| | | | | | lowering medication, use of anti- |
| | | | | | hypertensive medication, diabetes |
| | | | | | status, total cholesterol, high- |
| | | | | | density lipoprotein cholesterol and |
| | | | | | glomerular filtration rate adjusted |

### Example 3

The key finding of this study is that the measurement and incorporation of beta-2-microglobulin, cystatin C and C-reactive protein (CRP) into baseline risk models of cardiovascular disease and death significantly improved risk reclassification and discrimination in a high-risk group of patients undergoing coronary angiography. The inventors have discovered that all three biomarkers predict all-cause and cardiovascular mortality risk even when adjusting for a wide range of potential confounding factors. Importantly, these biomarkers predicted risk in a multi-ethnic cohort of both genders among individuals both with and without angiographic evidence of coronary artery disease, suggesting broad applicability in patients being considered for catheterization.

Novel treatment approaches have had a dramatic impact on cardiovascular outcomes over the last 30 years, with an approximate 30% reduction in cardiovascular mortality today compared to one generation ago¹⁹. However, cardiovascular disease remains by far the leading killer in the United States, suggesting that many at-risk patients remain unidentified and untreated²⁰. Clearly, novel methods to detect those at highest risk are desired.

Historical risk-prediction algorithms have largely focused on ‘traditional’ risk factors, incorporating the risk associated with comorbidities that have been related to cardiovascular disease through epidemiological association studies (e.g. smoking, hypertension, dyslipidemia, etc.)²¹. However, it is now known that these established risk factors account for only a fraction of one's lifetime risk of developing cardiovascular disease, with the balance being accounted for by other genetic and/or environmental factors which remain unidentified or unmeasured²². To better prognosticate risk of future events, other biochemical markers that reflect perturbations in disease-related pathways that are independent of classical risk factors will need to be identified.

To this end, the inventors have previously identified circulating beta-2-microglobulin as a factor strongly linked to both the presence and severity of peripheral arterial disease⁵. The inventors assessed if this major histocompatibility complex-associated polypeptide is shed from cells in response to hypoxia, given its noncovalent association with the cell membrane, it might be elevated in individuals with atherosclerotic disease. Beta-2-microglobulin has been reported to be associated with other vascular phenotypes²³, and has been associated with clinical outcomes in several lower risk cohorts²⁴⁻²⁶. As disclosed herein, the inventors have discovered that by associating elevated beta-2-microglobulin with cystatin C and C-reactive protein, reduced long-term survival due to both all-cause and cardiovascular mortality in a high-risk cohort. The use of these biomarkers is conceptually attractive in that it may reflect derangements in three different pathological pathways including ischemia-reperfusion injury (beta-2-microglobulin)²⁷, renal insufficiency (cystatin C)²⁸ and inflammation (C-reactive protein)²⁹.

Individuals referred for coronary angiography are among the highest risk patients encountered in cardiovascular medicine. The inventors assessed if additional stratification of this high-risk cohort leads to more effective and appropriate interventions while offering useful prognostic information to the individual patient. Finally, the inventors discovery that these biomarkers predict mortality risk regardless of whether or not significant CAD is identified during angiography is a very important point, as they may capture microvascular dysfunction that cannot be appreciated on an angiogram.

As the inventors examined the biomarkers in a high-risk group, the findings were are not generalizable to lower risk populations. Additionally, reliance on patient report to define the cause of death potentially introduced error into the cardiovascular mortality analysis and limited the ability to ascertain the cause of death in all cases. These biomarkers can be used in combination with other biomarkers which can be found subsequently in a confirmatory cohort or larger.

### Example 4

As disclosed in Examples 1-3, the inventors have demonstrated using an agnostic, mass spectrometry-based approach to identify proteomic makers which are dysregulated in those with PAD compared to those without (Fung et al., Vasc Med. 2008; 13: 217-24). This panel of biomarkers is correlated with PAD status, regardless of whether or not the patient also has CAD. Despite the clinical value of these biomarkers, the inventors enhanced this analysis with identification of a polymorphism in subjects to perfectly identify those at risk and/or having a PAD diagnosis. Human genetics studies have suggested that genetic factors may account for up to half of one's lifetime risk of cardiovascular disease (Marenberg et al., N Engl J Med. 1994; 330: 1041-6) and several recent studies report an association between polymorphisms (different to the rs10757269) at the non-coding chromosome 9p21 locus and cardiovascular diseases (Helgadottir et al., Science. 2007; 316: 1491-3, McPherson et al., Science. 2007; 316: 1488-91, Helgadottir et al., Nat Genet. 2008; 40: 217-24).

Accordingly, as disclosed herein, the inventors identified subjects with PAD that combines both classical risk factors with circulating biomarkers and genomic factors and also demonstrate that this risk prediction technique improves clinically relevant discriminatory indices, such as the integrated discrimination index and net reclassification index.

In particular, the inventors measured the genotype of the chromosome 9p21 cardiovascular-risk polymorphism rs10757269 as well as the proteomic biomarkers C-reactive protein, cystatin C and beta-2-microglobulin and plasma glucose in a study population of 393 patients undergoing coronary angiography. The rs10757269 allele was associated with PAD status (ankle-brachial index<0.9) independent of proteomic biomarkers and traditional cardiovascular risk factors (odds ratio=1.92; 95% confidence interval, 1.29-2.85). Importantly, compared to a previously validated risk factor-based PAD prediction model, the addition of proteomic biomarkers and rs10757269 significantly and incrementally improved PAD risk prediction as assessed by the net reclassification index (NRI, p=0.001) and integrated discrimination improvement (IDI, p=0.017).

Accordingly, the inventors demonstrate using a panel of biomarkers, which includes both genomic information (which is reflective of heritable risk) including the rs10757269 allele and proteomic information (which integrates environmental exposures), predicts the presence or absence of PAD better than prior established risk models, demonstrating the clinical utility for the diagnosis of PAD.

The baseline characteristics of the study population are presented in Table 11A. Genotype frequencies are presented in Table 11B.

TABLE 11A
Baseline study population characteristics (n = 393)

| | Value | |
|---|---|---|
| Characteristic | | |
| Age, mean years (SD) | 68 | (10) |
| Female, No.^{†} (%) | 180 | (46) |
| Ethnicity | | |
| Caucasian | 267 | (68) |
| African-American | 92 | (23) |
| Asian-American | 34 | (9) |
| Systolic blood pressure, mean mmHg (SD) | 140 | (21) |
| Body mass index, mean kg/m (SD) | 29 | (6) |
| Lipids, mean mg/Dl (SD) | | |
| Total cholesterol | 144 | (38) |
| High-density lipoprotein cholesterol | 42 | (13) |
| Current smoker, No. (%) | 43 | (11) |
| Use of cholesterol lowering medication, No. (%) | 255 | (65) |
| Use of antihypertensive therapy, No. (%) | 328 | (84) |
| Use of insulin or oral hypoglycemic, No. (%) | 115 | (29) |
| Ankle-brachial index, mean (SD) | 0.92 | (0.23) |
| History of cerebrovascular disease, No. (%) | 27 | (7) |
| History of congestive heart failure, No. (%) | 29 | (8) |
| History or coronary artery disease, No. (%) | 180 | (46) |
| Biomarker levels, median (IQR) | | |
| β₂-microglobulin | 1.9 | (1.5-2.6) |
| Cystatin C | 0.72 | (0.62-0.91) |
| C-reactive protein | 1.6 | (0.6-4.2) |
| Plasma glucose | 89 | (80-101) |

*SD, standard deviation;
^{†}No., number

TABLE 11B
Genotype distribution of rs10757269 by race, presented as No. (%)

| | GG | AG | AA |
|---|---|---|---|
| Caucasian | 83 (0.31) | 129 (0.48) | 55 (0.21) |
| African-American | 63 (0.68) | 23 (0.25) | 6 (0.07) |
| Asian-American | 19 (0.56) | 14 (0.41) | 1 (0.03) |

The inventors discovered that the G-allele of rs10757269 was associated with a significantly increased risk of PAD (Table 12). A statistically significant 80% increased risk of PAD per rs10757269 risk-allele remained even when accounting for risk factors and biomarkers previously shown to predict PAD. Accordingly, rs10757269 was also associated with a significantly decreased ABI per rs10757269 PAD risk increasing allele.

TABLE 12
Association of rs10757269 with peripheral arterial
disease and the ankle-brachial index.

| PAD | | ABI | | |
|---|---|---|---|---|
| OR (95% CI) | P-value | Coefficient (SE) | P-value | Adjustments* |
| 1.75 (1.27, 2.40) | 0.001 | −0.05 (0.02) | 0.002 | Age, gender, race |
| 1.91 (1.35, 2.71) | <0.001 | −0.05 (0.02) | 0.002 | Risk factors |
| 1.80 (1.25, 2.60) | 0.002 | −0.04 (0.01) | 0.012 | Risk factors and biomarkers |

OR, Odds ratio; CI, Confidence interval; SE, Standard error.
*Adjustment Risk factors include current smoking, body mass index, age, gender, race, diabetes, hypertension, total cholesterol, high-density lipoprotein cholesterol, lipid-lowering and antihypertensive medications; biomarkers include β₂−microglobulin, cystatin C, C-reactive protein and plasma glucose.

Additionally, the rs10757269 G-allele was associated with worse Walking Impairment Questionnaire distance, speed and stair climbing scores (Table 13). The inventors discovered that the G-allele predicted a statistically significant reduction in the Walking Impairment Questionnaire walking distance and stair-climbing scores even when adjusting for a wide range of PAD risk factors.

TABLE 13
shows the Association of rs0757269 with the Walking Impairment
Questionaire category scores.

| Measurement | Coefficient (SE) | P-value | Adjustments* |
|---|---|---|---|
| Walking Distance | −0.16 (0.07) | 0.025 | Age, gender, race |
| | −0.17 (0.07) | 0.011 | Risk factors |
| Stair-climbing | −0.15 (0.07) | 0.029 | Age, gender, race |
| | −0.16 (0.06) | 0.013 | Risk factors |
| Walking speed | −0.11 (0.07) | 0.112 | Age, gender, race |
| | −0.12 (0.06) | 0.055 | Risk factors |

SE, Standard error.
*Risk factors include current smoking, body mass index, age, gender, race, diabetes, hypertension, total chlesterol, high-density lipoprotein cholesterol, lipid-lowering and antihypertensive medications; biomarkers include β₂-microglobulin, cystatin C, C-reactive protein and plasma glucose

As rs10757269 was independently associated with PAD, the inventors next examined whether the addition of rs10757269 to a validated PAD risk factors model could improve risk discrimination and reclassification (Table 14). Table 14 shows the Integrated Discrimination Improvement (IDI) and Net Reclassification Index (NRI) for the addition of rs10757269 to established risk factors (e.g., the biomarkers β₂-microglobulin, cystatin C, C-reactive protein and plasma glucose). The addition of rs10757269 to the established risk factors model significantly improved the IDI. Similarly, a significant improvement in the IDI was seen with the addition of the biomarkers β2-microglobulin, cystatin C, C-reactive protein and plasma glucose, which have previously been shown to predict PAD. Interestingly, a significant improvement in model risk discrimination was still seen with the addition of rs10757269 to a baseline model including both established risk factors and biomarkers (IDI=0.016; P=0.017).

TABLE 14
The IDI and NRI for the addition of rs10757269 to established risk factors (IDI,
Integrated Discrimination Improvement; NRI, Net Reclassification Index; SE, Standard
Error, *Risk factors include age, gender, race/ethnicity, smoking status, BMI,
hypertension stage, diabetes status, and history of CAD, CVD, or CHF¹⁵; biomarkers
include β₂-microglobulin, cystatin C, C-reactive protein and plasma glucose)

| IDI | | NRI | | | | | |
|---|---|---|---|---|---|---|---|
| Estimate | P- | | | Non- | P- | | |
| (SE) | value | Estimate | Event | event | value | Baseline model* | Plus* |
| 0.020 | 0.006 | 31.25% | 3.90% | 27.35% | 0.003 | Risk factors | rs10757269 |
| (0.007) | | | | | | | |
| 0.040 | >0.001 | 48.63% | 5.66% | 42.97% | >0.001 | Risk factors | Biomarkers |
| (0.011) | | | | | | | |
| 0.014 | 0.033 | 33.88% | 9.09% | 24.79% | 0.001 | Risk factors and | rs10757269 |
| (0.007) | | | | | | biomarkers | |

Finally, the inventors assessed whether rs10757269 could improve PAD risk reclassification using the category free NRI. The inventors surprisingly discovered that both rs10757269 and the biomarkers were separately able to improve risk reclassification when added to the baseline model of established PAD risk factors. Importantly, rs10757269 was able to improve model risk reclassification even when added to a baseline model consisting of established risk factors and biomarkers (NRI=33.5%; P=0.001).

### Example 5

New methods to identify subjects with PAD are needed, as patients with this disease remain both underdiagnosed and undertreated (Hirsch et al., JAMA. 2001; 286: 1317-24. Nead et al., J Am Coll Cardiol. In press). The inventors demonstrate herein an assay integrate both a subject's genomic and proteomic information into currently available PAD risk prediction models, and thus improve the capacity to accurately identify those at risk. The inventors herein demonstrate that 1) both the 9p21 cardiovascular-risk allele and a panel of circulating biomarkers are associated with the presence of PAD as well as with walking ability, 2) these associations are independent of traditional cardiovascular risk factors, and 3) a combined model, which simultaneously measures a subject's genotype, clinical data, and biomarker status, provides superior risk discrimination and net reclassification capacity over established models, and may therefore have clinical utility.

Duval, et al. have reported a nomogram that assigns point values to traditional risk factors including age, gender, race, BMI, current smoking status, degree of hypertension, and presence or absence of diabetes, CAD, CVD, or CHF to create an evidence-based PAD risk score (Duval et al., Vasc Med. 2012; 17: 342-51). Although easy to administer, this score lacks a clearly defined threshold for PAD that exhibits both high sensitivity and specificity, suggesting the need for more discriminating risk factors. Moreover, it is now appreciated that traditional risk factors account for only half of one's lifetime risk of cardiovascular disease (Meijer et al., Arch Intern Med. 2000; 160: 2934-8), suggesting that the balance is accounted for by genetic and environmental factors which may not be captured in classical risk factor-based models. Accordingly, herein the inventors have pursued circulating biomarkers and genetic risk factors as an approach to quantify this ‘unmeasured risk’. The inventors demonstrated herein in Examples 1-3 that a panel of agnostically-identified proteomic biomarkers that is associated with PAD, and which improves mortality risk prediction (Fung, et al., Vasc Med. 2008; 13: 217-24, Nead, et al., Am J Cardiol. 2013; 111: 851-6). Genetic risk factors for PAD have been more difficult to ascertain due to the limitations of candidate-gene studies and the modest effect size of individual gene contributions to polygenic atherosclerotic disease (Leeper et al., Circulation. 2012; 125: 3220-8, Knowles et al., Arterioscler Thromb Vasc Biol. 2007; 27: 2068-78, Zintzaras et al., Am J Epidemiol. 2009; 170: 1-11). While genome-wide association studies have identified an association between different polymorphisms (e.g., not rs10757269) in the non-coding 9p21 chromosome region and low ankle-brachial index (ABI) (Murabito et al., Circ Cardiovasc Genet. 2012; 5: 100-12), or presence of polymorphisms rs1333049 or rs10757278 in 9p21 chromosome region associated with PAD (Cluett et al., Circ Cardiovasc Genet. 2009; 2: 347-53), they have not demonstrated that the rs10757269polymorphism is useful to identify at-risk populations for PAD. However, the association between 9p21 genotype and PAD is inconstant (Helgadottir et al., Nat Genet. 2008; 40: 217-2, Murabito et al., Circ Cardiovasc Genet. 2012; 5: 100-12, Cluett et al., Circ Cardiovasc Genet. 2009; 2: 347-53), indicating that it cannot be relied upon by itself to identify the presence of PAD.

Herein, the inventors have surprisingly discovered that the combination of biomarkers (beta-2-microglobulin, cystatin C, C-reactive protein and plasma glucose) and genetic markers (SNPs at the 9p21 locus) are independently associated with PAD and more importantly, provide additive improvements in risk discrimination and risk reclassification. The inventors have demonstrated the independence of the biomarkers and the 9p21 SNP likely reflect their correlation with distinct pathways related to atherogenesis in the periphery. Circulating biomarkers provide a ‘readout’ of activated disease-related metabolic pathways, and incorporate a subject's recent exposure to environmental factors which may alter the epigenetic, transcriptional or translational regulation of a given pathway. The panel employed in this study has relevance to PAD, as it may simultaneously contribute information about the subject's current level of peripheral ischemia-reperfusion injury, renal dysfunction and vascular inflammation (Fung et al., Vasc Med. 2008; 13: 217-24). The 9p21 status, on the other hand, is a genetic risk factor that signifies a potentially fixed, lifelong exposure. SNPs at the 9p21 locus are known to correlate with disease independent of traditional risk factors, and are represent a novel aspect of the vascular biology responsible for disease initiation or progression. Recent work by the inventors demonstrates that variation in the 9p21 locus may accelerate smooth muscle cell apoptosis and alter the integrity of the developing neointimal lesion (data not shown), demonstrating how the rs10757269 polymorphism promotes risk regardless of whether a patient also happens to be hypertensive or dyslipidemic (Leeper et al., Arterioscler Thromb Vasc Biol. 2013; 33: e1-e10).

In some embodiments, the use of the biomarkers and rs10757269 polymorphism as disclosed herein as markers for risk of PAD can apply to the general population (e.g., multi-ethnic population). In some embodiments, the use of the biomarkers and rs10757269 polymorphism as disclosed herein as markers for risk of PAD can apply to a specific ethnic group, or certain race subgroups and racial groups.

As disclosed herein, the inventors demonstrate a model (e.g., a combination of biomarkers and rs10757269 polymorphism) which predicts baseline PAD. Accordingly, the inventors are the first to integrate genomic and proteomic information for diagnosis of PAD, which has been demonstrated to enhance the capacity to identify PAD disease which is highly prevalent and significantly underdiagnosed and is responsible for approximately every fifth dollar spent on inpatient cardiovascular care in the United States (Mahoney et al., Circ Cardiovasc Qual Outcomes. 2008; 1: 38-45). Accordingly, the present invention enables a quicker and more reliable diagnosis of subjects at risk of PAD, thus allowing intervening therapeutic action and/or improved health care and/or lifestyle changes by the subject to attempt to overt the occurrence PAD, which results in decreased health care costs long term.

### REFERENCES

The references cited herein and throughout the background and specification are incorporated herein in their entirety by reference.
1. Ambrose J A, Srikanth S. Vulnerable plaques and patients: improving prediction of future coronary events. Am J Med 2010; 123:10-16.
2. Wilson A M, Kimura E, Harada R K, Nair N, Narasimhan B, Meng X Y, Zhang F, Beck K R, Olin J W, Fung E T, Cooke J P. Beta2-microglobulin as a biomarker in peripheral arterial disease: proteomic profiling and clinical studies. Circulation 2007; 116:1396-1403.
3. Sadrzadeh Rafie A H, Stefanick M L, Sims S T, Phan T, Higgins M, Gabriel A, Assimes T, Narasimhan B, Nead K T, Myers J, Olin J, Cooke J P. Sex differences in the prevalence of peripheral artery disease in patients undergoing coronary catheterization. Vasc Med 2010; 15:443-450.
4. Wilson A M, Sadrzadeh-Rafie A H, Myers J, Assimes T, Nead K T, Higgins M, Gabriel A, Olin J, Cooke J P. Low lifetime recreational activity is a risk factor for peripheral arterial disease. J Vasc Surg 2011; 54:427-432, 432 e421-424.
5. Fung E T, Wilson A M, Zhang F, Harris N, Edwards K A, Olin J W, Cooke J P. A biomarker panel for peripheral arterial disease. Vasc Med 2008; 13:217-224.
6. Levey A S, Bosch J P, Lewis J B, Greene T, Rogers N, Roth D. A more accurate method to estimate glomerular filtration rate from serum creatinine: a new prediction equation. Modification of Diet in Renal Disease Study Group. Ann Intern Med 1999; 130:461-470.
7. Tonino P A, Fearon W F, De Bruyne B, Oldroyd K G, Leesar M A, Ver Lee P N, Maccarthy P A, Van't Veer M, Pijls N H. Angiographic versus functional severity of coronary artery stenoses in the FAME study fractional flow reserve versus angiography in multivessel evaluation. J Am Coll Cardiol 2010; 55:2816-2821.
8. Atar D, Ramanujam P S, Saunamaki K, Haunso S. Assessment of coronary artery stenosis pressure gradient by quantitative coronary arteriography in patients with coronary artery disease. Clin Physiol 1994; 14:23-35.
9. McGeechan K, Macaskill P, Irwig L, Liew G, Wong T Y. Assessing new biomarkers and predictive models for use in clinical practice: a clinician's guide. Arch Intern Med 2008; 168:2304-2310.
10. D'Agostino R B, Sr., Vasan R S, Pencina M J, Wolf P A, Cobain M, Massaro J M, Kannel W B. General cardiovascular risk profile for use in primary care: the Framingham Heart Study. Circulation 2008; 117:743-753.
11. Henry R M, Kostense P J, Bos G, Dekker J M, Nijpels G, Heine R J, Bouter L M, Stehouwer C D. Mild renal insufficiency is associated with increased cardiovascular mortality: The Hoorn Study. Kidney Int 2002; 62:1402-1407.
12. Conroy R M, Pyorala K, Fitzgerald A P, Sans S, Menotti A, De Backer G, De Bacquer D, Ducimetiere P, Jousilahti P, Keil U, Njolstad I, Oganov R G, Thomsen T, Tunstall-Pedoe H, Tverdal A, Wedel H, Whincup P, Wilhelmsen L, Graham I M. Estimation of ten-year risk of fatal cardiovascular disease in Europe: the SCORE project. Eur Heart J 2003; 24:987-1003.
13. Stamler J, Vaccaro O, Neaton J D, Wentworth D. Diabetes, other risk factors, and 12-yr cardiovascular mortality for men screened in the Multiple Risk Factor Intervention Trial. Diabetes Care 1993; 16:434-444.
14. Pencina M J, D'Agostino R B, Sr., D'Agostino R B, Jr., Vasan R S. Evaluating the added predictive ability of a new marker: from area under the ROC curve to reclassification and beyond. Stat Med 2008; 27:157-172; discussion 207-112.
15. Pencina M J, D'Agostino R B, Sr., Steyerberg E W. Extensions of net reclassification improvement calculations to measure usefulness of new biomarkers. Stat Med 2011; 30:11-21.
16. Harrell F E, Jr., Lee K L, Mark D B. Multivariable prognostic models: issues in developing models, evaluating assumptions and adequacy, and measuring and reducing errors. Stat Med 1996; 15:361-387.
17. Pencina M J, D'Agostino R B. Overall C as a measure of discrimination in survival analysis: model specific population value and confidence interval estimation. Stat Med 2004; 23:2109-2123.
18. Harris P A, Taylor R, Thielke R, Payne J, Gonzalez N, Conde J G. Research electronic data capture (REDCap)—a metadata-driven methodology and workflow process for providing translational research informatics support. J Biomed Inform 2009; 42:377-381.
19. Capewell S, Morrison C E, McMurray J J. Contribution of modern cardiovascular treatment and risk factor changes to the decline in coronary heart disease mortality in Scotland between 1975 and 1994. Heart 1999; 81:380-386.
20. Roger V L, Go A S, Lloyd-Jones D M, Benjamin E J, Berry J D, Borden W B, Bravata D M, Dai S, Ford E S, Fox C S, Fullerton H J, Gillespie C, Hailpern S M, Heit J A, Howard V J, Kissela B M, Kittner S J, Lackland D T, Lichtman J H, Lisabeth L D, Makuc D M, Marcus G M, Marelli A, Matchar D B, Moy C S, Mozaffarian D, Mussolino M E, Nichol G, Paynter N P, Soliman E Z, Sorlie P D, Sotoodehnia N, Turan T N, Virani S S, Wong N D, Woo D, Turner M B. Executive summary: heart disease and stroke statistics—2012 update: a report from the American Heart Association. Circulation 2012; 125:188-197.
21. Dent T H. Predicting the risk of coronary heart disease I. The use of conventional risk markers. Atherosclerosis 2010; 213:345-351.
22. Leeper N J, Kullo I J, Cooke J P. Genetics of peripheral artery disease. Circulation 2012; 125:3220-3228.
23. Kals J, Zagura M, Serg M, Kampus P, Zilmer K, Unt E, Lieberg J, Eha J, Peetsalu A, Zilmer M. beta2-microglobulin, a novel biomarker of peripheral arterial disease, independently predicts aortic stiffness in these patients. Scand J Clin Lab Invest 2011; 71:257-263.
24. Amighi J, Hoke M, Mlekusch W, Schlager 0, Exner M, Haumer M, Pernicka E, Koppensteiner R, Minar E, Rumpold H, Schillinger M, Wagner 0. Beta 2 microglobulin and the risk for cardiovascular events in patients with asymptomatic carotid atherosclerosis. Stroke 2011; 42:1826-1833.
25. Shinkai S, Chaves P H, Fujiwara Y, Watanabe S, Shibata H, Yoshida H, Suzuki T. Beta2-microglobulin for risk stratification of total mortality in the elderly population: comparison with cystatin C and C-reactive protein. Arch Intern Med 2008; 168:200-206.
26. Astor B C, Shafi T, Hoogeveen R C, Matsushita K, Ballantyne C M, hiker L A, Coresh J. Novel markers of kidney function as predictors of ESRD, cardiovascular disease, and mortality in the general population. Am J Kidney Dis 2012; 59:653-662.
27. Kalawski R, Majewski M, Kaszkowiak E, Wysocki H, Siminiak T. Transcardiac release of soluble adhesion molecules during coronary artery bypass grafting: effects of crystalloid and blood cardioplegia. Chest 2003; 123:1355-1360.
28. Coll E, Botey A, Alvarez L, Poch E, Quinto L, Saurina A, Vera M, Piera C, Darnell A. Serum cystatin C as a new marker for noninvasive estimation of glomerular filtration rate and as a marker for early renal impairment. Am J Kidney Dis 2000; 36:29-34.
29. Pearson T A, Mensah G A, Alexander R W, Anderson J L, Cannon R O, 3rd, Criqui M, Fadl Y Y, Fortmann S P, Hong Y, Myers G L, Rifai N, Smith S C, Jr., Taubert K, Tracy R P, Vinicor F. Markers of inflammation and cardiovascular disease: application to clinical and public health practice: A statement for healthcare professionals from the Centers for Disease Control and Prevention and the American Heart Association. Circulation 2003; 107:499-511.
30. Criqui M H, Fronek A, Barrett-Connor E, Klauber M R, Gabriel S and Goodman D. The prevalence of peripheral arterial disease in a defined population. Circulation. 1985; 71: 510-5.
31. Hirsch A T, Criqui M H, Treat-Jacobson D, et al. Peripheral arterial disease detection, awareness, and treatment in primary care. JAMA. 2001; 286: 1317-24.
32. Meijer W T, Hoes A W, Rutgers D, Bots M L, Hofman A and Grobbee D E. Peripheral arterial disease in the elderly: The Rotterdam Study. Arterioscler Thromb Vasc Biol. 1998; 18: 185-92.
33. McDermott M M, Mehta S, Ahn H and Greenland P. Atherosclerotic risk factors are less intensively treated in patients with peripheral arterial disease than in patients with coronary artery disease. J Gen Intern Med. 1997; 12: 209-15.
34. Anand S S, Kundi A, Eikelboom J and Yusuf S. Low rates of preventive practices in patients with peripheral vascular disease. Can J Cardiol. 1999; 15: 1259-63.
35. Oka R K, Umoh E, Szuba A, Giacomini J C and Cooke J P. Suboptimal intensity of risk factor modification in PAD. Vasc Med. 2005; 10: 91-6.
36. Valentijn™ and Stolker R J. Lessons from the REACH Registry in Europe. Curr Vasc Pharmacol. 2012; 10: 725-7.
37. Steg P G, Bhatt D L, Wilson P W, et al. One-year cardiovascular event rates in outpatients with atherothrombosis. JAMA. 2007; 297: 1197-206.
38. Bhatt D L, Flather M D, Hacke W, et al. Patients with prior myocardial infarction, stroke, or symptomatic peripheral arterial disease in the CHARISMA trial. J Am Coll Cardiol. 2007; 49: 1982-8.
39. Murabito J M, D'Agostino R B, Silbershatz H and Wilson W F. Intermittent claudication. A risk profile from The Framingham Heart Study. Circulation. 1997; 96: 44-9.
40. Pradhan A D, Shrivastava S, Cook N R, Rifai N, Creager M A and Ridker P M. Symptomatic peripheral arterial disease in women: nontraditional biomarkers of elevated risk. Circulation. 2008; 117: 823-31.
41. Wilson A M, Kimura E, Harada R K, et al. Beta2-microglobulin as a biomarker in peripheral arterial disease: proteomic profiling and clinical studies. Circulation. 2007; 116: 1396-403.
42. Leeper N J, Kullo I J and Cooke J P. Genetics of peripheral artery disease. Circulation. 2012; 125: 3220-8.
43. Hamburg N M and Leeper N J. Therapeutic potential of modulating microRNA in peripheral arterial disease. Curr Vasc Pharmacol. In press.
44. Duval S, Massaro J M, Jaff M R, et al. An evidence-based score to detect prevalent peripheral artery disease (PAD). Vasc Med. 2012; 17: 342-51.
45. Fung E T, Wilson A M, Zhang F, et al. A biomarker panel for peripheral arterial disease. Vasc Med. 2008; 13: 217-24.
46. Marenberg M E, Risch N, Berkman L F, Floderus B and de Faire U. Genetic susceptibility to death from coronary heart disease in a study of twins. N Engl J Med. 1994; 330: 1041-6.
47. Helgadottir A, Thorleifsson G, Manolescu A, et al. A common variant on chromosome 9p21 affects the risk of myocardial infarction. Science. 2007; 316: 1491-3.
48. McPherson R, Pertsemlidis A, Kavaslar N, et al. A common allele on chromosome 9 associated with coronary heart disease. Science. 2007; 316: 1488-91.
49. Helgadottir A, Thorleifsson G, Magnusson K P, et al. The same sequence variant on 9p21 associates with myocardial infarction, abdominal aortic aneurysm and intracranial aneurysm. Nat Genet. 2008; 40: 217-24.
50. Wilson A M, Sadrzadeh-Rafie A H, Myers J, et al. Low lifetime recreational activity is a risk factor for peripheral arterial disease. J Vase Surg. 2011; 54: 427-32, 32 e1-4.
51. Sadrzadeh Rafie A H, Stefanick M L, Sims S T, et al. Sex differences in the prevalence of peripheral artery disease in patients undergoing coronary catheterization. Vasc Med. 2010; 15: 443-50.
52. Nead K T, Zhou M J, Caceres R D, et al. Usefulness of the addition of beta-2-microglobulin, cystatin C and C-reactive protein to an established risk factors model to improve mortality risk prediction in patients undergoing coronary angiography. Am J Cardiol. 2013; 111: 851-6.
53. Creager M A, Belkin M, Bluth E I, et al. 2012 ACCF/AHA/ACR/SCAI/SIR/STS/SVM/SVN/SVS Key data elements and definitions for peripheral atherosclerotic vascular disease: a report of the American College of Cardiology Foundation/American Heart Association Task Force on Clinical Data Standards (Writing Committee to develop Clinical Data Standards for peripheral atherosclerotic vascular disease). J Am Coll Cardiol. 2012; 59: 294-357.
54. Ding H, Xu Y, Wang X, et al. 9p21 is a shared susceptibility locus strongly for coronary artery disease and weakly for ischemic stroke in Chinese Han population. Circ Cardiovasc Genet. 2009; 2: 338-46.
55. Heckman M G, Soto-Ortolaza A I, Diehl N N, et al. Genetic variants associated with myocardial infarction in the PSMA6 gene and Chr9p21 are also associated with ischaemic stroke. Eur J Neurol. 2013; 20: 300-8.
56. Shiffman D, O'Meara E S, Rowland C M, et al. The contribution of a 9p21.3 variant, a KIF6 variant, and C-reactive protein to predicting risk of myocardial infarction in a prospective study. BMC Cardiovasc Disord. 2011; 11: 10.
57. Murabito J M, White C C, Kavousi M, et al. Association between chromosome 9p21 variants and the ankle-brachial index identified by a meta-analysis of 21 genome-wide association studies. Circ Cardiovasc Genet. 2012; 5: 100-12.
58. Nead K T, Zhou M, Diaz Caceres R, Olin J W, Cooke J P and Leeper N J. The Walking Impairment Questionnaire improves mortality risk prediction models in a high-risk cohort independent of peripheral arterial disease status. Circ Cardiovasc Qual Outcomes. In press.
59. McDermott M M, Liu K, Guralnik J M, Martin G J, Criqui M H and Greenland P. Measurement of walking endurance and walking velocity with questionnaire: validation of the walking impairment questionnaire in men and women with peripheral arterial disease. J Vasc Surg. 1998; 28: 1072-81.
60. Regensteiner J G, Steiner J F and Hiatt W R. Exercise training improves functional status in patients with peripheral arterial disease. J Vasc Surg. 1996; 23: 104-15.
61. Pencina M J, D'Agostino R B, Sr., D'Agostino R B, Jr. and Vasan R S. Evaluating the added predictive ability of a new marker: from area under the ROC curve to reclassification and beyond. Stat Med. 2008; 27: 157-72; discussion 207-12.
62. Harris P A, Taylor R, Thielke R, Payne J, Gonzalez N and Conde J G. Research electronic data capture (REDCap)—a metadata-driven methodology and workflow process for providing translational research informatics support. J Biomed Inform. 2009; 42: 377-81.
63. Nead K T, Olin J W, Cooke J P and Leeper N J. Alternative ankle-brachial index method identifies additional at-risk individuals. J Am Coll Cardiol. In press.
64. Fowkes F G, Housley E, Macintyre C C, Prescott R J and Ruckley C V. Variability of ankle and brachial systolic pressures in the measurement of atherosclerotic peripheral arterial disease. J Epidemiol Community Health. 1988; 42: 128-33.
65. Meijer W T, Grobbee D E, Hunink M G, Hofman A and Hoes A W. Determinants of peripheral arterial disease in the elderly: the Rotterdam study. Arch Intern Med. 2000; 160: 2934-8.
66. Knowles J W, Assimes T L, Li J, Quertermous T and Cooke J P. Genetic susceptibility to peripheral arterial disease: a dark corner in vascular biology. Arterioscler Thromb Vasc Biol. 2007; 27: 2068-78.
67. Zintzaras E and Zdoukopoulos N. A field synopsis and meta-analysis of genetic association studies in peripheral arterial disease: The CUMAGAS-PAD database. Am J Epidemiol. 2009; 170: 1-11.
68. Cluett C, McDermott M M, Guralnik J, et al. The 9p21 myocardial infarction risk allele increases risk of peripheral artery disease in older people. Circ Cardiovasc Genet. 2009; 2: 347-53.
69. Leeper N J, Raiesdana A, Kojima Y, et al. Loss of CDKN2B promotes p53-dependent smooth muscle cell apoptosis and aneurysm formation. Arterioscler Thromb Vasc Biol. 2013; 33: e1-e10.
70. Mahoney E M, Wang K, Cohen D J, et al. One-year costs in patients with a history of or at risk for atherothrombosis in the United States. Circ Cardiovasc Qual Outcomes. 2008; 1: 38-45.

1.-30. (canceled)

31. A method of identifying a subject at risk of a major adverse event, the method comprising detecting in a biological sample taken from the subject presenting a symptom of an acute cardiac event, or BMI of 25-30 or greater than 30, for the level of at least three biomarkers selected from beta-2-microglobulin, c-reactive protein (CRP) and cystatin C, wherein combination of the levels of beta-2-microglobulin, c-reactive protein (CRP) and cystatin C equal to, or above a threshold reference level for each of beta-2-microglobulin, c-reactive protein (CRP) and cystatin C indicates that the subject is at risk of a major adverse event.

32. A method of identifying a subject suitable for treatment to prevent the occurrence of a major adverse event, the method comprising detecting in a biological sample taken from the subject presenting a symptom of an acute cardiac event, or BMI of 25-30 or greater than 30, for the level of at least three biomarkers selected from beta-2-microglobulin, c-reactive protein (CRP) and cystatin C, wherein the combination of the levels of beta-2-microglobulin, c-reactive protein (CRP) and cystatin C above threshold reference levels for each beta-2-microglobulin, c-reactive protein (CRP) and cystatin C indicates that the subject should undergo treatment to reduce the incidence of a major adverse event.

33. The method of claim 31, wherein the levels of beta-2-microglobulin, c-reactive protein (CRP) and cystatin C are measured in a biological sample obtained from a subject who has fasted.

34. The method of claim 31, wherein the biological sample is a blood-based biological sample, or urine sample.

35. The method of claim 31, wherein the levels of beta-2-microglobulin, c-reactive protein (CRP) and cystatin C are measured using an antibody, antibody fragment or protein-binding molecule or other protein-binding probe.

36. The method of claim 31, wherein the antibody, antibody fragment or protein-binding molecule or other protein-binding probe is bound to a solid support.

37. The method of claim 31, wherein the levels of beta-2-microglobulin, c-reactive protein (CRP) and cystatin C are measured using an immunoassay.

38. The method of claim 37, wherein the immunoassay is an ELISA.

39. The method of claim 31, wherein the subject is a Caucasian subject.

40. The method of claim 31, wherein the subject is a Black, Hispanic or Asian subject.

41. The method of claim 31, wherein the subject is of Asian-Indian, Pakistani, Middle Eastern or Pacific Islander ethnicity.

42. The method of claim 31, wherein a treatment to prevent the occurrence a major adverse event is selected from the group of: an exercise program; control of blood pressure, reduced sugar intake, cessation of smoking and drug therapies selected from the group of aspirin (with or without dipyridamole), clopidogrel, cilostazol, and/or pentoxifylline.

43. (canceled)

44. A method for treating a human subject with a risk of a major adverse event, comprising administering a treatment or therapy to prevent the occurrence of a major adverse event to a human subject who is determined to have a level of beta-2-microglobulin, c-reactive protein (CRP) and cystatin C equal to, or above a reference threshold level for each biomarker.

45. The method of claim 44, wherein the treatment or therapy to prevent the occurrence a major adverse event is selected from the group consisting of: an exercise program; control of blood pressure, reduced sugar intake, cessation of smoking and drug therapies selected from the group of aspirin (with or without dipyridamole), clopidogrel, cilostazol, and/or pentoxifylline.

46. The method of claim 44, wherein the major adverse event is stroke, heart attack or death.

47. The method of claim 44, wherein the major adverse event is a major adverse cardiovascular or cerebrovascular event (MACCE).

48. The method of claim 47, wherein the MACCE is selected from the group consisting of: recurrence of an initial cardiac event, angina, decompensation of heart failure, admission for cardiovascular disease (CVD), mortality due to CVD, and transplant.

49. The method of claim 44, wherein the reference threshold level for beta-2-microglobulin is 1.88 mg/l.

50. The method of any of claim 44, wherein the reference threshold level for CRP is 1.60 mg/l.

51. The method of claim 44, wherein the reference threshold level for cystatin C is 0.72 mg/l.


SEQUENCE LISTING (1 sequence)

SEQ ID NO: 1 - DNA, 52 bp, Homo sapiens
cttaattccttgataggttcttttagrtaatttttttataatgaagcaatta